(19) United States
(12) Patent Application Publication
(10) Pub. No.: US 2022/0397576 A1
(43) Pub. Date: Dec. 15, 2022

(54) APPARATUSES AND METHODS FOR DETECTION OF PANCREATIC CANCER

(71) Applicant: RECCAN DIAGNOSTICS AB, Lund (SE)

(72) Inventors: Daniel Ansari, Lund (SE); Roland Andersson, Lund (SE); György Marko-Varga, Malmö (SE)

(21) Appl. No.: 17/770,025

(22) PCT Filed: Oct. 15, 2020
(86) PCT No.: PCT/SE2020/050984

§ 371 Date: Apr. 18, 2022

Related U.S. Application Data
(60) Provisional application No. 62/923,336, filed on Oct. 18, 2019.

(30) Foreign Application Priority Data
Oct. 18, 2019 (SE) 1930339-5
May 15, 2020 (SE) 2030164-4

(51) Int. Cl. G01N 33/574; G01N 33/543

(52) U.S. Cl. CPC G01N 33/57438; G01N 33/57484; G01N 33/54388

(57) ABSTRACT

A pancreatic cancer detection device, including: a solid surface comprising an antibody bound to the solid surface, the solid surface configured to indicate selective binding between the antibody and one or more target protein(s); and wherein the antibody is configured to selectively bind to the target protein(s). In some examples, the target protein(s) include one or more protein(s) selected from the group consisting of Alpha-1 antitrypsin (A1AT), Alpha-1-acid glycoprotein 1 (AGP1), Apolipoprotein A1 (ApoA1), C1-inhibitor, Complement C2, Complement component 3, Carbohydrate antigen 19-9, Calprotectin, caspase-cleaved cytokeratin-18 (CCK18), Ceruloplasmin, cartilage oligomeric matrix protein, gamma-glutamyl transpeptidase, Haptoglobin, Insulin-like growth factor 1, Insulin-Like Growth Factor Binding Protein 3, Properdin, Serum amyloid A, and Tumor necrosis factor alpha (TNF alpha).

### FIELD

This application relates to devices and methods for the detection of biomarkers indicative of pancreatic cancer, wherein the biomarkers are detected in biological samples from a patient.

### BACKGROUND

Pancreatic cancer is an almost uniformly fatal disease. Tremendous efforts have been made to elucidate the mechanisms underlying pancreatic cancer in order to develop effective treatments. Although there have been significant scientific advancements, pancreatic cancer survival rates remain stagnant with a 5-year survival rate of 9%. In the United States, 56,770 patients are predicted to be diagnosed with pancreatic cancer and 45,750 individuals will die from the disease in 2019 (Siegel R L, et al. CA Cancer J Clin 2019; 69(1):7-34). Despite the continuous overall decline in the death rates from most cancer forms, both incidence and mortality rates for pancreatic cancer have increased during the past decade (Wu W, et al. Clin Epidemiol 2018; 10:789-97). It is projected that pancreatic cancer will become the second leading cause of cancer related death by the year 2030 (Rahib L, et al. Cancer Res 2014; 74(11):2913-21).

Surgical resection is the only curative treatment option, yet only about 15-20% of patients are eligible for up-front radical surgery (Kommalapati A, et al. Cancers (Basel) 2018; 10(1)). Early detection of resectable tumors is key to reduce pancreatic cancer related deaths (Lennon A M. et al. Cancer Res 2014; 74(13):3381-9). Apart from early diagnosis, molecular markers are also needed to accurately predict the course of the disease or response to therapy (Krantz B A et al. Clin Cancer Res 2018; 24(10):2241-2250). Serum CA 19-9 is the only biomarker used in the routine clinical management of pancreatic cancer. However, CA 19-9 has inadequate sensitivity and specificity for early detection and can only be used for disease monitoring (Poruk K E, et al. Curr Mol Med 2013; 13(3):340-51). Consequently, to improve patient outcomes, novel and improved diagnostic, prognostic and predictive biomarkers are needed to identify instances of pancreatic cancer and to characterize individual pancreatic tumor biology for the selection of optimal treatment.

### SUMMARY

Some aspects relate to a pancreatic cancer detection device, including:
a solid surface comprising an antibody bound to the solid surface, the solid surface configured to indicate selective binding between the antibody and a target protein; and
wherein the antibody is configured to selectively bind to the target protein selected from the group consisting of Alpha-1 antitrypsin (A1AT), Alpha-1-acid glycoprotein 1 (AGP1), Apolipoprotein A1 (ApoA1), C1-inhibitor, Complement C2, Complement component 3, Carbohydrate antigen 19-9, Calprotectin, caspase-cleaved cytokeratin-18 (CCK18), Ceruloplasmin, cartilage oligomeric matrix protein, gamma-glutamyl transpeptidase, Haptoglobin, Insulin-like growth factor 1, Insulin-Like Growth Factor Binding Protein 3, Properdin, Serum amyloid A, and Tumor necrosis factor alpha (TNF alpha).

In some examples, the solid surface includes antibodies configured to selectively bind two target proteins selected from the group.

In some examples, the solid surface includes antibodies configured to selectively bind three target proteins selected from the group.

In some examples, the solid surface includes antibodies configured to selectively bind four target proteins selected from the group.

In some examples, the solid surface includes antibodies configured to selectively bind five target proteins selected from the group.

In some examples, the solid surface comprises antibodies configured to selectively bind A1AT and Carbohydrate antigen 19-9.

Where the solid surface comprises antibodies configured to selectively bind A1AT and Carbohydrate antigen 19-9, the solid surface may comprise antibodies further configured to selectively bind at least one of Complement C2 and Complement component 3. The solid surface may comprise antibodies further configured to selectively bind cartilage oligomeric matrix protein. The solid surface may comprise antibodies further configured to selectively bind at least one of Gamma-glutamyl transpeptidase, C1 inhibitor, and Serum amyloid A.

In some examples, the solid surface comprises antibodies configured to selectively bind A1AT, Carbohydrate antigen 19-9, at least one of Complement C2 and Complement component 3, and Gamma-glutamyl transpeptidase. The solid surface may comprise antibodies further configured to selectively bind at least one of C1 inhibitor, and Serum amyloid A.

In some examples, the solid surface comprises antibodies configured to selectively bind A1AT, Carbohydrate antigen 19-9, and Gamma-glutamyl transpeptidase. The solid surface may comprise antibodies further configured to selectively bind cartilage oligomeric matrix protein. The solid surface may comprise antibodies further configured to selectively bind Serum amyloid A. In some examples, the solid surface comprises antibodies configured to selectively bind A1AT, Carbohydrate antigen 19-9, and cartilage oligomeric matrix protein. The solid surface may comprise antibodies further configured to selectively bind Serum amyloid A.

In some examples, the solid surface comprises antibodies configured to selectively bind Carbohydrate antigen 19-9 and at least one of Complement C2 and Complement component 3.

In some examples, the solid surface includes a lateral flow detection surface.

In some examples, the lateral flow detection surface includes a lateral flow detection test trip.

In some examples, indicating includes visually indicating binding to a user.

Some aspects relate to a method of detecting pancreatic cancer, including:
collecting a biological sample from a subject;
contacting the biological sample with the pancreatic detection device of any one of the preceding claims; and
indicating a likelihood of an incidence of pancreatic cancer in the subject.

In some examples, the biological sample includes whole blood.

In some examples, the biological sample includes serum.

In some examples, the method further includes providing a report, the report indicating the likelihood of an incidence of pancreatic cancer in a subject.

Some aspects relate to a pancreatic cancer detection device, including:
a solid surface comprising an antibody bound to the solid surface, the solid surface configured to indicate selective binding between the antibody and a target protein; and
wherein the antibody is configured to selectively bind to the target protein selected from the group consisting of Claudin 18 (CLDN18), Galectin 4 (LGALS4), Matrix metalloproteinase 7 (MMP7), Mucin 2 (MUC2), Mucin 4 (MUC4), Olfactomedin 4 (OLFM4), Regenerating islet-derived protein 1-alpha (REG1A), Regenerating islet-derived protein 1-beta (REG1B), Serine protease inhibitor Kazal-type 1 (SPINK1), Syncollin (SYCN), Trefoil factor 1 (TFF1), Carcinoembryonic antigen (CEA), Lymphatic vessel endothelial hyaluronan receptor 1 (LYVE1), Tenascin C (TNC), and Thrombospondin 2 (THBS2).

In some examples, the solid surface comprises antibodies configured to selectively bind two target proteins selected from the group.

In some examples, the solid surface comprises antibodies configured to selectively bind three target proteins selected from the group.

In some examples, the solid surface comprises antibodies configured to selectively bind four target proteins selected from the group.

In some examples, the solid surface comprises antibodies configured to selectively bind five target proteins selected from the group.

Some aspects relate to a method of developing a protein expression profile in a biological sample obtained from a patient, the method including:
detecting and quantifying a level of one or more fragment peptides in a protein digest prepared from the biological sample using mass spectrometry; and
calculating a level of a corresponding protein or proteins in the biological sample;
wherein the one or more corresponding protein or proteins is selected from the group consisting of Apolipoprotein A-I, Immunoglobulin lambda-like polypeptide 5, Alpha-2-HS-glycoprotein, Immunoglobulin lambda constant 2, Alpha-1-acid glycoprotein 1, Immunoglobulin heavy constant gamma 1, Immunoglobulin kappa constant, Immunoglobulin heavy constant alpha 1, Serotransferrin, Serum albumin, Alpha-1-antitrypsin, Brain acid soluble protein 1, Protein S100-A6, Collagen alpha-1(XIV) chain, Histone H1.5, Fibulin-1, Rho GDP-dissociation inhibitor 2, Phospholipase A2, Pancreatic triacylglycerol lipase, Chymotrypsin-like elastase family member 3A, Colipase, Bile salt-activated lipase, Trypsin-2, Carboxypeptidase A1, Protein disulfide-isomerase A2, Trypsin-1, Glutathione S-transferase A2, D-3-phosphoglycerate dehydrogenase, Polyadenylate-binding protein 4, Protein disulfide-isomerase, Translocon-associated protein subunit alpha, Y-box-binding protein 3, Ribosome-binding protein 1, Leucine-rich repeat-containing protein 59, Protein disulfide-isomerase A4, 78 kDa glucose-regulated protein, Hypoxia up-regulated protein 1, N(G), N(G)-dimethylarginine dimethylaminohydrolase 1, Elongation factor 1-beta, Phosphatidylethanolamine-binding protein 1, 40S ribosomal protein S21, 40S ribosomal protein S3a, 40S ribosomal protein S14, 60S ribosomal protein L12, and Protein TFG.

In some examples, the method further includes the step of fractionating the protein digest prior to detecting and quantifying the amount of the one or more fragment peptides.

In some examples, the fractionating step includes liquid chromatography, nano-reverse phase liquid chromatography, high performance liquid chromatography or reverse phase high performance liquid chromatography.

In some examples, the biological is a fresh or a fresh-frozen sample.

In some examples, the biological sample is a formalin fixed tissue.

In some examples, the protein digest includes a protease digest.

In some examples, the protein digest includes a trypsin digest.

In some examples, the mass spectrometry includes tandem mass spectrometry, ion trap mass spectrometry, triple quadrupole mass spectrometry, hybrid ion trap/quadrupole mass spectrometry, MALDI-TOF mass spectrometry, MALDI mass spectrometry, and/or time of flight mass spectrometry.

In some examples, a mode of mass spectrometry used is Selected Reaction Monitoring (SRM), Multiple Reaction Monitoring (MRM), intelligent Selected Reaction Monitoring (iSRM), Parallel Reaction Monitoring (PRM), and/or multiple Selected Reaction Monitoring (mSRM).

In some examples, the one or more fragment peptides are selected from the group consisting of peptides corresponding to SEQ ID NO: 1-46.

In some examples, the formalin fixed tissue is paraffin embedded tissue.

In some examples, the tissue is obtained from a tumor.

In some examples, the tumor is a primary tumor.

In some examples, the tumor is a secondary tumor.

In some examples, quantifying the one or more fragment peptides includes comparing an amount of the one or more fragment peptides in the biological sample to the amount of the same one or more fragment peptides in a different and separate biological sample.

In some examples, quantifying the one or more fragment peptides includes determining an amount of the one or more fragment peptides in the biological sample by comparison to an added internal standard peptide of known amount having the same amino acid sequence of the one or more fragment peptides.

In some examples, the internal standard peptide is an isotopically labeled peptide.

In some examples, the isotopically labeled internal standard peptide comprises one or more heavy stable isotopes selected from the group consisting of 180, 170, 34S, 15N, 13C, 2H and a combination thereof.

In some examples, detecting and quantifying the amount of the one or more fragment peptides in the protein digest indicates the presence of the corresponding protein and an association with cancer in the subject.

In some examples, the method further includes administering to a patient or subject from which the biological sample was obtained a therapeutically effective amount of a cancer therapeutic agent, wherein the cancer therapeutic agent and/or amount of the cancer therapeutic agent administered is based upon detection of and/or amount of the one or more proteins or the one or more fragment peptides selected from SEQ ID NO: 1-46, and wherein the cancer therapeutic agent is a targeted agent that interacts with the one or more proteins that correspond to the one or more fragment peptides selected from SEQ ID NO: 1-46.

In some examples, the cancer therapeutic agent and/or amount of the cancer therapeutic agent administered is based upon multiplex detection of and/or amount of two or more fragment peptides selected from SEQ ID NO: 1-46.

In some examples, the method further includes administering to a patient or subject from which the biological sample was obtained a therapeutically effective amount of a cancer therapeutic agent, wherein the cancer therapeutic agent and/or amount of the cancer therapeutic agent administered is based upon detection of and/or amount of the one or more protein or the one or more fragment peptides selected from SEQ ID NO: 1-46, and wherein the cancer therapeutic agent is an immunomodulatory cancer therapeutic agent whose function is to initiate, enhance, manipulate, and/or otherwise modulate the cancer patient immune response to attack and kill said patient tumor cells.

In some examples, the method further includes combining multiplex detecting and quantitating two or more proteins or two or more fragment peptides corresponding to SEQ ID NO: 1-46 with analysis of other oncoproteins that drive growth of the patient tumor cells, wherein a targeted cancer therapeutic agent that inhibits or modulates the function of the oncoprotein to inhibit growth of the patient tumor cells is administered to the patient in combination with an immunomodulatory cancer therapeutic agent that interacts with one or more of the proteins to initiate, enhance, manipulate, and/or otherwise modulate the cancer patient immune response to attack and kill the patient tumor cells.

Some aspects relate to a method of determining if a subject has an increased risk of suffering from pancreatic cancer, the method including
a) analyzing at least one sample from the subject to determine a value of the subject's proteomic profile, and
b) comparing the value of the subject's proteomic profile with the value obtained from subjects determined to define a normal proteomic profile, to determine if the subject's proteomic profile is altered compared to a normal proteomic profile,
wherein a change in the value of the subject's proteomic profile is indicative that the subject has an increased risk of suffering from future pancreatic cancer compared to those defined as having a normal proteomic profile.

In some examples, the normal proteomic profile includes the subject's proteomic profile prior to the onset of pan.

In some examples, the normal proteomic profile includes a proteomic profile generated from a population of individuals that do not presently or in the future display memory impairment.

Some aspects relate to a method of monitoring the progression of pancreatic cancer in a subject, the method including
a) analyzing at least two samples from the subject with each sample taken at different time points to determine the values of each of the subject's proteomic profile, and
b) comparing the values of the subject's proteomic profile over time to determine if the subject's proteomic profile is changing over time,
wherein a change in the subject's proteomic profile over time is indicative that the subject's risk of suffering from pancreatic cancer is increasing over time.

Some aspects relate to a method of monitoring the progression of a treatment for pancreatic cancer in a subject, the method including:
a) analyzing at least two samples from a subject undergoing treatment for pancreatic cancer with each sample taken at different time points to determine the values of each of the subject's proteomic profile, and
b) comparing the values of the subject's proteomic profile over time to determine if the subject's proteomic profile is changing over time in response to the treatment,
wherein a lack of change or a further deviation from a normal proteomic profile in the subject's proteomic profile is indicative that the treatment for pancreatic cancer is not effective, and wherein an approximation of the subject's proteomic profile over time towards a normal proteomic profile is indicative that the treatment for pancreatic cancer is effective in treating pancreatic cancer in the subject.

Some aspects relate to a method of determining if a subject has an increased risk of suffering from pancreatic cancer, the method including analyzing at least one sample from the subject to determine levels of individual proteins and comparing the levels of individual proteins with the value of levels of the proteins in one or more normal individuals to determine if the levels of each protein are altered compared to normal levels, wherein a change in the value of the subject's proteins is indicative that the subject has an increased risk of suffering from pancreatic cancer compared to a normal individual.

### BRIEF DESCRIPTION OF THE DRAWINGS

FIG. 1 depicts an example of a lateral flow detection device.

FIG. 2 depicts an example of a methodological workflow.

FIGS. 3 (A)-(D) depict the results of an example Mass Spectrometry (MS) discovery study. (A) Principle component 1 plotted vs. Principle component 2. (B) Upregulated and downregulated genes. (C) Statistical analysis of Fold changes. (D) Top-ranked proteins, including BASP1.

FIG. 4 (A)-(D) depict the results of an example targeted proteomics study and bioinformatic analysis of candidate protein biomarkers. (A) consensus clustering heatmap. (B) BASP1 is one of the most reproducible candidates. (C) pathway analysis. (D Mapping of the differentially expressed proteins into the BASP1/WT1 network.

FIGS. 5 (A) and (B) depict the results of example Immunohistochemistry (IHC) and Immunofluorescence (IF) analysis of BASP1/WT1.

FIG. 6 (a)-(f) depict the results of an example survival analysis of BASP1/WT1 in tissue microarray samples.

FIG. 7 (a)-(e) depict the results of an example subgroup analyses of BASP1/WT1 expression and their correlation with overall survival (OS) in pancreatic cancer patients.

FIG. 8 depicts example results comparing pancreatic cancer sera vs. healthy control sera.

### DETAILED DESCRIPTION

### Lateral Flow Devices

Provided herein are lateral flow assay devices and methods of using such devices to detect biomarkers for pancreatic cancer in samples from a subject. One of skill in the art will understand that such lateral flow assay devices may be used to detect any of the biomarkers described herein, for example, such lateral flow assay devices may be used to detect any suitable combination of biomarkers described herein and provide an indication of an instance of pancreatic cancer. Further, although lateral flow devices are described in detail herein, one of skill in the art will understand that other types of devices and systems may be suitable for the detection of pancreatic cancer, therefore this disclosure is not limited to the use of a lateral flow device.

The term “immobilized” or “embedded” interchangeably refers to reversibly or irreversibly immobilized molecules (e.g., analytes or binding agents). In some examples, reversibly immobilized molecules are immobilized in a manner that allows the molecules, or a portion thereof (e.g., at least about 25%, 50%, 60%, 75%, 80% or more of the molecules), to be removed from their immobilized location without substantial denaturation or aggregation. For example, a molecule can be reversibly immobilized in or on an absorbent material (e.g., an absorbent pad) by contacting a solution containing the molecule with the absorbent material, thereby soaking up the solution and reversibly immobilizing the molecule. The reversibly immobilized molecule can then be removed by wicking the solution from the absorbent material, or from one region of the absorbent material to another. In some cases, a molecule can be reversibly immobilized on an absorbent material by contacting a solution containing the molecule with the absorbent material, thereby soaking up the solution, and then drying the solution-containing absorbent material. The reversibly immobilized molecule can then be removed by contacting the absorbent material with another solution of the same or a different composition, thereby solubilizing the reversibly immobilized molecule, and then wicking the solution from the absorbent material, or from one region of the absorbent material to another.

Irreversibly immobilized molecules (e.g., binding agents or analytes) are immobilized such that they are not removed, or not substantially removed, from their location under mild conditions (e.g., pH between about 4-9, temperature of between about 4-65° C.). Exemplary irreversibly immobilized molecules include protein analytes or binding agents bound to a nitrocellulose, polyvinylidene fluoride, nylon or polysulfone membrane by standard blotting techniques (e.g., electroblotting). Other exemplary irreversibly immobilized molecules include protein analytes or binding agents bound to glass or plastic (e.g., a microarray, a microfluidic chip, a glass histology slide or a plastic microtiter plate having wells with bound protein analytes therein).

The term “binding agent” refers to an agent that specifically binds to a molecule such as an analyte. While antibodies are described in many contexts herein, it will be understood by one of skill in the art that other binding agents can be used instead of antibodies as preferred by the user. A wide variety of binding agents are known in the art, including antibodies, aptamers, affimers, lipocalins (e.g., anticalins), thioredoxin A, bilin binding protein, or proteins containing an ankyrin repeat, the Z domain of staphylococcal protein A, or a fibronectin type III domain. Other binding agents include, but are not limited to, biotin/streptavidin, chelating agents, chromatography resins, affinity tags, or functionalized beads, nanoparticles and magnetic particles.

The term “specifically bind” refers to a molecule (e.g., binding agent such as an antibody or antibody fragment) that binds to a target with at least 2-fold greater affinity than non-target compounds, e.g., at least about 4-fold, 5-fold, 6-fold, 7-fold, 8-fold, 9-fold, 10-fold, 20-fold, 25-fold, 50-fold, 100-fold, 1000-fold, or more than 1000-fold greater affinity.

The term “antibody” refers to a polypeptide comprising a framework region from an immunoglobulin gene, or fragments thereof, that specifically bind and recognize an antigen, e.g., a particular analyte. Typically, the “variable region” contains the antigen-binding region of the antibody (or its functional equivalent) and is most critical in specificity and affinity of binding. Antibodies include for example chimeric, human, humanized antibodies, or single-chain antibodies.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

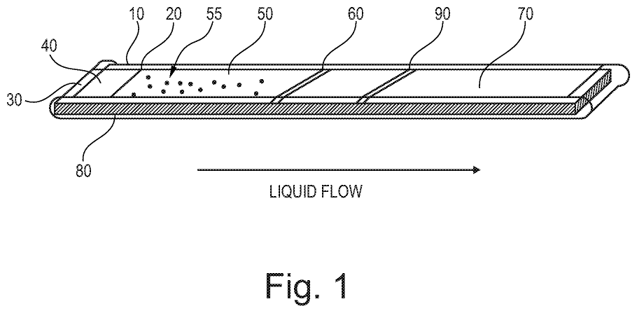

A example lateral flow device for detecting pancreatic cancer is shown in FIG. 1, which shows a schematic diagram of a device with an elongated housing 10 that contains a lateral flow strip 20. The lateral flow strip 20 may extend substantially the entire length of housing 10. The lateral flow strip 20 may be divided into a sample application area 40 positioned below an optional sample introduction port 30, an antigen-antibody conjugation site 50, a capture area 60, and a distal absorbent pad 70. The antigen-antibody conjugation site 50 can have mobile antigens 55. The flow strip 20 can also have a backing 80. The mobile antigen 55 in the antigen-antibody conjugation site 50 can be labeled antigens (such as gold-conjugated antigen) that can react with and bind to antibodies in a test sample from a subject. A flow path along the lateral flow strip 20 passes from the sample application area 40, through the antigen-antibody conjugation site 50, into the capture area 60. Immobilized binding entities such as one or more antibodies that recognize one or more proteins correlated with pancreatic cancer, are positioned on capture area 60. Alternatively, the mobile antigens 55 can bind one or more antibodies that may be present in a test sample and the liquid flow can transport a conjugate formed between a mobile antigen and an antibody to the capture area 60, where immobilized binding entities can capture the antigen-antibody conjugates and concentrate the label in the capture area 60. The mobile antigens 55 without a bound antibody pass through the capture area 60 and are eventually collected in the distal absorbent pad 70. The lateral flow strip 20 can also include a reaction verification or control area 90. Such a control area 90 (e.g., configured as line) can be slightly distal to the capture area 60. The reaction verification or control area 90 illustrates to a user that the test has been performed. Prior to the test being performed, the reaction verification or control area 90 is not visible. However, when the test is performed by placing a fluid sample on the sample application area 40, the reaction verification or control area 90 can become visible as the sample flows through the capture area 60 and to the distal absorbent pad 70. For example, the reaction verification or control area 90 can become visible due to a chemical reacting with any component of the sample or simply due to the presence of moisture in the sample.

Using the methods described herein, one or more lateral flow device(s) may be used to detect one or more protein selected from the group consisting of Apolipoprotein A-I, Immunoglobulin lambda-like polypeptide 5, Alpha-2-HS-glycoprotein, Immunoglobulin lambda constant 2, Alpha-1-acid glycoprotein 1, Immunoglobulin heavy constant gamma 1, Immunoglobulin kappa constant, Immunoglobulin heavy constant alpha 1, Serotransferrin, Serum albumin, Alpha-1-antitrypsin, Brain acid soluble protein 1, Protein S100-A6, Collagen alpha-1(XIV) chain, Histone H1.5, Fibulin-1, Rho GDP-dissociation inhibitor 2, Phospholipase A2, Pancreatic triacylglycerol lipase, Chymotrypsin-like elastase family member 3A, Colipase, Bile salt-activated lipase, Trypsin-2, Carboxypeptidase A1, Protein disulfide-isomerase A2, Trypsin-1, Glutathione S-transferase A2, D-3-phosphoglycerate dehydrogenase, Polyadenylate-binding protein 4, Protein disulfide-isomerase, Translocon-associated protein subunit alpha, Y-box-binding protein 3, Ribosome-binding protein 1, Leucine-rich repeat-containing protein 59, Protein disulfide-isomerase A4, 78 kDa glucose-regulated protein, Hypoxia up-regulated protein 1, N(G), N(G)-dimethylarginine dimethylaminohydrolase 1, Elongation factor 1-beta, Phosphatidylethanolamine-binding protein 1, 40S ribosomal protein S21, 40S ribosomal protein S3a, 40S ribosomal protein S14, 60S ribosomal protein L12, and Protein TFG. Either the complete set of these proteins or a subset thereof may be detected. A subset may include any combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of these proteins.

More specifically and as described in greater detail below (including example methods of proteomic profiling to identify such biomarkers), 18 biomarkers found in blood were identified as particularly strong indicators for the detection of pancreatic cancer: A1AT (Alpha-1 antitrypsin), AGP1 (Alpha-1-acid glycoprotein 1), ApoA1 (Apolipoprotein A1), C1 inhib (C1-inhibitor, C1-inh, C1 esterase inhibitor), C2 (Complement C2), C3 (Complement component 3), CA19-9 (Carbohydrate antigen 19-9), Calprotectin CCK18 (caspase-cleaved cytokeratin-18), Ceruloplasmin, COMP (cartilage oligomeric matrix protein), GT (gamma-glutamyl transpeptidase), Haptoglobin, IGF1 (IGF-1, Insulin-like growth factor 1), IGFB3 (IGFBP-3, Insulin-Like Growth Factor Binding Protein 3), Properdin, SAA (Serum amyloid A), and TNF-a (Tumor necrosis factor alpha). As will be understood by one of skill in the art, detection of any subcombination of less than all 18 of these biomarkers may still provide reliable detection of pancreatic cancer. For example, combinations of 2, 3, 4, 5, 6, 7, 8, 9, 10, 11, 12, 13, 14, 15, 16, 17, 18 of these biomarkers may be used in an aforementioned lateral flow assay or any other suitable assay to detect pancreatic cancer and notify a user. For example, a smaller subset of 10 biomarkers from the list of 18 biomarkers found in blood may also provide reliable detection of pancreatic cancer, IGFBP3, AGP1, GT, COMP, C1 inhibitor, C3, ApoA1, IGF1, CCK18, CA 19-9. Such a combination has been found to provide a diagnostic accuracy of 100% against healthy controls and a diagnostic accuracy of 90.5% against benign pancreatic disease. In certain examples of a detection assay, a subcombination of 5 biomarkers may be used, such as: A1AT, TNF-alpha, AGP1, C2, CA 19-9. Such a combination has been found to provide a diagnostic accuracy of 100% against healthy controls and a diagnostic accuracy of 79.6% against benign pancreatic disease. In certain examples of a detection assay, a subcombination of 4 biomarkers may be used, such as A1AT, TNF-alpha, AGP1, and CA 19-9. This 4 biomarker combination has been found to provide a diagnostic accuracy of 99.9% against healthy controls and a diagnostic accuracy of 76% against benign pancreatic disease. In certain examples of a detection assay, a subcombination of 3 biomarkers may be used, such as ApoA1, SAA, and CA 19-9. This three marker has been found to provide a a diagnostic accuracy of 93% against healthy controls and a diagnostic accuracy of 77% against benign pancreatic disease. As will be understood by one of skill in the art, smaller combinations of biomarkers may be more easily deployed in a detection assay, such as a lateral flow assay.

A person skilled in the art will appreciate that a number of methods can be used to detect or quantify the DNA/RNA/protein levels of various disease- or health-related biomarkers.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP.

A person skilled in the art will appreciate that a number of methods can be used to detect or quantify the level of RNA products of the disease- or health-related biomarkers within a sample, including arrays, such as microarrays, RT-PCR (including quantitative PCR), nuclease protection assays and Northern blot analyses.

### Centralized Testing

Centralized testing platforms may be used to combine fluidics, optics, and digital signal processing with microsphere technology to deliver multiplexed assay capabilities to perform protein or nucleic acid assays quickly, cost-effectively, and accurately. For example, the Luminex (Austin, Tex.) xMAP® Technology is a centralized testing platform that enables multiplexing of biological tests (assays), reducing time, labor, and costs over traditional methods such as ELISA, western blotting, PCR, and traditional arrays. For example, such systems may perform discrete assays on the surface of color-coded beads known as microspheres, which are then read in a compact analyzer. Using multiple lasers or LEDs and high-speed digital-signal processors, the analyzer reads multiplex assay results by reporting the reactions occurring on each individual microsphere.

Due to robust multiplexing, centralized testing potentially delivers more data in less time than other bioassay products, with comparable results to ELISA and microarray. Centralized testing several other distinct advantages over traditional methods including (a) Speed/High Throughput—because each microsphere serves as an individual test, a large number of different bioassays can be performed and analyzed simultaneously; (b) Versatility—a centralized testing system can perform bioassays in several different formats, including nucleic acids and antigen-antibody binding, along with enzyme, receptor-ligand, and other protein interactions; (c) Flexibility—the technology can be customized for the user's specific needs or updated periodically by attaching a specific probe to a uniquely colored microsphere; (d) Accuracy—the technology generates real-time analysis and accurate quantification of the biological interactions; and (e) Reproducibility-high volume production of microspheres within a single lot allows assay standardization that solid-phased planar arrays cannot provide.

Centralized testing platforms both a high plex capability and high throughput at the same time. Traditional ELISA, real-time PCR and other technologies that excel at high throughput applications (greater than 1000 samples per day) lack the ability to multiplex more than five tests at a time. On the other hand, microarray technology excels in high density screening (greater than 250-plex tests), but lacks the reproducibility needed for high throughput applications. For applications requiring a throughput of up to 1000 samples per day and multiplexing from one to 500 tests per sample.

### Proteomic Profiling

Various methods exist in the art for profiling the biomarkers associated with a particular disease state, for example pancreatic cancer. One of skill in the art will understand that the methods and techniques described below may be used to characterize a number of disease states.

Selected reaction monitoring (SRM) is a method used in tandem mass spectrometry in which an ion of a particular mass is selected in the first stage of a tandem mass spectrometer and an ion product of a fragmentation reaction of the precursor ion is selected in the second mass spectrometer stage for detection (E. de Hoffmann (1996) Journal of Mass Spectrometry. 31(2): 129-137). Multiple reaction monitoring (MRM) is the application of selected reaction monitoring to multiple product ions from one or more precursor ions (Murray, et al. (2013) Pure and Applied Chemistry. 85 (7): 1515-1609; and Kondrat, R. W. et al. (1978) Analytical Chemistry. 50(14): 2017-2021).

Parallel reaction monitoring (PRM) is an ion monitoring technique based on high-resolution and high-precision mass spectrometry. The principle of this technique is comparable to SRM/MRM, but it is more convenient in assay development for absolute quantification of proteins and peptides. It is most suitable for quantification of multiple proteins in complex samples with an attomole-level detection. PRM is based on Q-Orbitrap as the representative quadrupole-high resolution mass spectrum platform. Unlike SRM, which performs one transition at a time, PRM performs a full scan of each transition by a precursor ion, that is, parallel monitoring of all fragments from the precursor ion. PRM technology not only has the SRM/MRM target quantitative analysis capabilities, but also has the qualitative ability. The mass accuracy can reach to ppm level, which can eliminate the background interference and false positive better than SRM/MRM, and improves the detection limit and sensitivity in complex background effectively. It provides a full scan of product ions, without the need to select the ion pair and optimize the fragmentation energy, and it is easier to establish the assay. In addition, it provides a wider linear range: increased to 5-6 orders of magnitude.

Examples of methods are provided for carrying out parallel reaction monitoring (PRM) or specific mass spectrometry-SRM/MRM assays useful for developing a molecular profile for a patient, by precisely quantifying specific protease-digested peptides derived from a collection of proteins having a variety of functions and cellular locations in proteomic lysates prepared directly from patient tissue, e.g., a tumor tissue. The process and assays can be used for understanding the molecular landscape of a patient's tumor and to guide selection of optimal cancer therapeutic agents that either directly kill the tumor cells or induce, initiate, support, and/or otherwise manipulate an active and successful immune response to the patient's own tumor cells, leading to improved patient survival. Cells from a biological sample of a cancer patient, such as, for example, fresh tissues, fresh-frozen tissues, or formalin-fixed paraffin embedded (FFPE) tumor tissue, can be collected using, for example, the methodology of tissue microdissection. “Fresh-frozen” tissues for mass spectrometry analysis may include tumor specimens and normal pancreas controls. In some cases, evidence suggests superiority of fresh-frozen over FFPE for mass spectrometry (Bauden M, et al. Lab Invest. 2017 March; 97(3):279-288). However, fresh-frozen tissues may be rare and not easily attainable compared to FFPE. For tissue microarrays and immunohistochemistry, we have typically used FFPE.

A lysate for mass spectrometry analysis can be prepared from the collected cells using, for example, the Liquid Tissue® reagents and protocol (e.g. see U.S. Pat. No. 7,473,532). The lysate can be analyzed using PRM or specific SRM/MRM assays as described in more detail below, where the assays are performed individually or in multiplex, and using protein detection/quantitation data from these SRM/MRM assays to develop a molecular profile for the patient/subject. These methods and the resulting PRM or SRM/MRM assay data can be used to determine an improved or optimal treatment regimen for the patient using therapeutic agents that directly function to inhibit protein function to kill tumor cells and inhibit their growth. In addition, the PRM or SRM/MRM assay data can be used to determine an improved or optimal treatment regimen for the patient using therapeutic agents that function to initiate, modulate, effect, enhance, and/or otherwise manipulate the cancer patient immune system to kill the tumor cells by directly interacting with one or more of the proteins detected and/or quantitated by the presently described SRM/MRM assays.

Determining a patient molecular profile by the described PRM or SRM/MRM assays may be performed on a variety of patient-derived samples including but not limited to blood, urine, sputum, pleural effusion, inflammatory fluid surrounding a tumor, normal tissue, and/or tumor tissue. In a particular example, the sample is FFPE tissue, for example FFPE tumor tissue.

FFPE tissue is the most widely and advantageously available form of tissue, including tumor tissue, from cancer patients. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Molecular analytical methods that can analyze formalin fixed cancer tissue are the most accepted and heavily utilized methods for analysis of cancer patient tissue.

The most widely-used methodology presently applied to analyze protein expression in cancer patient tissue, especially FFPE tissue, is immunohistochemistry (IHC). IHC methodology uses an antibody to detect the protein of interest. The results of an IHC test are most often interpreted by a pathologist or histotechnologist. This interpretation is subjective and does not provide quantitative data that may be predictive of sensitivity to therapeutic agents that target specific proteins. Each pathologist running a test also may use different criteria to decide whether the results are positive or negative. In most cases, this happens when the test results are borderline, i.e. the results are neither strongly positive nor strongly negative. In other cases, cells present in one area of the cancer tissue section can test positive while cells in a different area of the cancer tissue section can test negative. Inaccurate test results may mean that patients diagnosed with cancer do not receive the best possible care. If all or a specific region/cells of tumor tissue is truly positive for a specific protein but test results classify it as negative, physicians are unlikely to administer the correct therapeutic treatment to the patient. If tumor tissue is truly negative for expression of a specified protein but test results classify it as positive, physicians may use a specific therapeutic treatment even though the patient is not only unlikely to receive any benefit but also will be exposed to the agent's secondary risks. Accordingly, there is great clinical value in the ability to precisely detect and correctly evaluate quantitative levels of specific proteins in tumor tissue so that the patient will have the greatest chance of receiving a successful treatment regimen while reducing unnecessary toxicity and other side effects.

Precise detection and correct evaluation of quantitative levels of specific proteins in tumor tissue may be effectively determined in a mass spectrometer by PRM or SRM/MRM methodology. This methodology detects and quantitates unique fragment peptides from specific proteins, including cancer biomarkers, in which the SRM/MRM signature chromatographic peak area of each peptide is determined within a complex peptide mixture present in a lysate. One method of preparing a complex biomolecule sample directly from formalin-fixed tissue is described in U.S. Pat. No. 7,473,532. In a particular example, the proteolytic enzyme trypsin may be used to fragment proteins in a sample. Quantitative levels of proteins can then be determined by the PRM or SRM/MRM methodology whereby the PRM or SRM/MRM signature chromatographic peak area of an individual specified peptide from each protein in a biological sample can be compared to the PRM or SRM/MRM signature chromatographic peak area of a known amount of a “spiked” internal standard for each of the individual fragment peptides.

In one example, the “spiked” internal standard is a synthetic version of the same exact protein-derived fragment peptide where the synthetic peptide contains one or more amino acid residues labeled with one or more heavy isotopes, such as ²H, ¹⁸O, ¹⁷O, ¹⁵N, ¹³C, or combinations thereof. Such isotope labeled internal standards are synthesized so that mass spectrometry analysis generates a predictable and consistent PRM or SRM/MRM signature chromatographic peak that is different and distinct from the native fragment peptide chromatographic signature peak and which can be used as comparator peak. Thus when the internal standard is “spiked” in known amounts into a protein or peptide preparation from a biological sample and analyzed by mass spectrometry, the PRM or SRM/MRM signature chromatographic peak area of the native peptide is compared to the PRM or SRM/MRM signature chromatographic peak area of the internal standard peptide, and this numerical comparison indicates either the absolute molarity and/or absolute weight of the native peptide present in the original proteomic preparation from the biological sample. Quantitative data for fragment peptides are displayed according to the amount of proteomic lysate analyzed per sample.

In order to develop and perform the PRM or SRM/MRM assay for a fragment peptide for a given protein, additional information beyond simply the peptide sequence may be utilized by the mass spectrometer. This additional information can be used to direct and instruct the mass spectrometer (e.g., a triple quadrupole mass spectrometer) to perform the correct and focused analysis of a specific fragment peptide. The additional information about a target peptide in general may include one or more of the mono isotopic mass of each peptide, its precursor charge state, the precursor m/z value, the m/z transition ions, and the ion type of each transition ion. A PRM or SRM/MRM assay may be effectively performed on a triple quadrupole mass spectrometer or an ion trap/quadrupole hybrid instrument. These types of mass spectrometers can analyze a single isolated target peptide within a very complex protein lysate containing hundreds of thousands to millions of individual peptides from all the proteins contained within a cell. This additional information provides the mass spectrometer with the correct directives to allow analysis of a single isolated target peptide within a very complex protein lysate. PRM or SRM/MRM assays also can be developed and performed on other types of mass spectrometer, including MALDI, ion trap, ion trap/quadrupole hybrid, or triple quadrupole instruments.

The foundation for a single PRM or SRM/MRM assay to detect and quantitate a specific protein in a biological sample is identification and analysis of one or more fragment peptides derived from the larger, full length version of the protein. This is because mass spectrometers are highly efficient, proficient, and reproducible instruments when analyzing very small molecules such as a single fragment peptide while mass spectrometers cannot efficiently, proficiently, or reproducibly detect and quantitate full length, intact proteins.

A candidate peptide for developing a single PRM or SRM/MRM assay for an individual protein may theoretically be any individual peptide that results from complete protease digestion, as for example digestion with trypsin, of the intact full length proteins. Many peptides are unsuitable for reliable detection and quantitation of any given protein—indeed, for some proteins no suitable peptide has yet been found. Accordingly, it is impossible to predict which is the most advantageous peptide to assay by PRM or SRM/MRM for a given protein, and therefore the specifically-defined assay characteristics about each peptide must be empirically discovered and determined. This is especially true when identifying the best PRM or SRM/MRM peptide for analysis in a protein lysate such as a lysate from FFPE tissue. The presently described PRM or SRM/MRM assays designate one or more protease digested peptides (e.g., tryptic digested peptides) for each protein whereby each peptide has been discovered to be an advantageous peptide for PRM or SRM/MRM assays.

The presently described PRM or SRM/MRM assays detect and quantitate proteins that can be used to develop a molecular profile of the patient tumor tissue microenvironment. These proteins provide a wide variety of functions and are found in a wide variety of locations within the cell. These proteins include, but are not limited to growth factors, growth factor receptors, extracellular matrix proteins, nuclear transcription factors, epithelial cell differentiation factors, cell signaling proteins, immune cell differentiation factors, cell/cell recognition factors, self vs. tumor recognition factors, immune cell activation factors, immune cell inhibiting factors, and immune checkpoint proteins. Each of these individual proteins within this collection of proteins can be, and are, expressed by a wide variety of cells in the cancer patient including, but not limited to, all varieties of solid tissue cells such as epithelial tumor cells, normal epithelial cells, normal fibroblasts, tumor-associated fibroblasts, normal endothelial cells, tumor-associated endothelial cells, normal mesenchymal cells, and tumor-associated mesenchymal cells. Each of these proteins can be expressed by a wide variety of blood-born white blood cells including but not limited to all varieties of lymphocytes, such as B cells, T cells, macrophages, dendrites, mast cells, natural killer cells, eosinophils, neutrophils, and basophils. It is well known that in many cases each of these individual proteins can be expressed by both solid tissue cells and blood-born tissue cells.

The presently described PRM or SRM/MRM assays detect and quantitate expression of unique proteins expressed by many different cell types demonstrating many different functions and residing in many different locations within the cell. Each of the assays describes at least one optimal peptide that was found to be useful for reliable and reproducible detection and measurement of a single protein, where each assay can be performed individually or in multiplex with other peptides for other proteins.

The peptides found in Tables 1 and 2 were derived from their respective designated proteins by protease digestion of all the proteins within a complex lysate prepared from cells procured from human tissue. The lysate was then analyzed by mass spectrometry to determine those peptides derived from a designated protein that are detected and analyzed by mass spectrometry. Identification of a specific preferred subset of peptides for mass spectrometric analysis is based on discovery under experimental conditions of which peptide or peptides from a protein ionize in mass spectrometry analyses of lysates, and thus demonstrate the ability of the peptide to result from the protocol and experimental conditions used in preparing a lysate to be analyzed by the methodology of mass spectrometry.

The method for measuring the level of a designated protein in a biological sample described herein (or fragment peptides as surrogates thereof) may be used as a diagnostic indicator of pancreatic cancer in a patient or subject. The results from measurements of the level of a designated protein may be employed to determine the diagnostic stage/grade/status of a pancreatic cancer by correlating (e.g., comparing) the level of the protein found in a tissue with the level of that protein found in normal and/or cancerous or precancerous tissues. The results from measurements of the level of a designated protein also may be employed to determine which cancer therapeutic agents to treat a pancreatic cancer patient with and thus the most optimal cancer treatment regimen.

The tissue protein expression landscape is highly complex whereby multiple proteins expressed by multiple types of solid tissue cells and localized/non-localized immune cells require multiple assays for multiple therapeutic agent indications. This level of protein assay complication can be analyzed by the presently described PRM or SRM/MRM assays. These assays are designed to substantially simultaneously (or at substantially the same time or substantially together) detect and quantify many different proteins having a variety of molecular functions, where the proteins include, but are not limited to soluble proteins, membrane-bound proteins, nuclear factors, differentiation factors, proteins that modulate cell-to-cell interactions, secreted proteins, immune checkpoint proteins, growth factors, growth factor receptors, cell signaling proteins, immune inhibitory proteins, cytokines, and lymphocyte-activating/inhibiting factors.

Tissue microdissection can advantageously be used to procure pure populations of tumor cells from patient tumor tissue for protein expression analysis using the PRM or SRM/MRM assays in order to determine the molecular profile that specifically defines tumor cell status for the patient. Tissue microdissection of tumor tissue can be performed using the process of laser induced forward transfer of cells and cell populations, e.g., utilizing DIRECTOR® technology.

The presently described PRM or SRM/MRM assays detect and quantitate expression of specific proteins in lysates prepared from solid tissue, e.g., tumor tissue. However, unless pure populations of cells are collected and analyzed these assays may not accurately provide detailed information about which cells express which proteins. In some cases, this is important because aberrant protein expression is common in the tumor microenvironment, as for example when tumor cells express immune inhibitory factors that are usually expressed solely by normal cells, normal lymphocytic cells, and/or tumor infiltrating lymphocytes (TILs). Thus, when expression of candidate therapeutic protein targets has been detected and quantified by the described PRM or SRM/MRM assays, a follow-up assay may be necessary to provide missing cellular localization information. The method to achieve cellular expression context is immunohistochemistry. Understanding which proteins are expressed within the tumor microenvironment and which cells express these proteins may advantageously inform optimal treatment decisions to modulate the patient's own immune response to seek out and kill the tumor cells. The presently described PRM or SRM/MRM assays and analysis process provide the ability to detect and quantify protein targets of cancer therapeutic agents directly in patient tumor tissue.

An advantageous approach for tumor cell killing is to use a combination therapy whereby immunomodulatory agents are used in combination with tumor cell targeting agents synergistically for optimal patient response. PRM or SRM/MRM assays can be used to determine the quantitative expression status in patient tumor tissue of oncoprotein targets for which inhibitory therapeutic agents have been developed.

In some examples, combining multiplex detecting and quantitating of two or more fragment peptides corresponding to SEQ ID NO: 1-46 with analysis of other oncoproteins that drive growth of the patient tumor cells can be advantageous. This can allow a targeted cancer therapeutic agent that inhibits or modulates the function of the oncoprotein to inhibit growth of the patient tumor cells to be administered to the patient in combination with an immunomodulatory cancer therapeutic agent that interacts with one or more of the proteins to initiate, enhance, manipulate, and/or otherwise modulate the cancer patient immune response to attack and kill the patient tumor cells.

Because both nucleic acids and protein can be analyzed from the same biomolecular preparation it is possible to generate additional information about drug treatment decisions from the nucleic acids in the same sample analyzed with the presently described PRM or SRM/MRM assays. A specific protein can be found by the presently described PRM or SRM/MRM assays to be expressed by certain cells at increased levels while at the same time information about the mutation status of specific genes and/or the nucleic acids and proteins they encode (e.g., mRNA molecules and their expression levels or splice variations) can be obtained. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

### Risk of Pancreatic Cancer

Disclosed herein are methods of determining if a subject has an increased risk of suffering from pancreatic cancer. The methods may comprise analyzing at least one plasma and/or blood sample from the subject to determine a value of the subject's proteomic profile and comparing the value of the subject's proteomic profile with the value of a normal proteomic profile. A change in the value of the subject's proteomic profile, over or under normal values is indicative that the subject has an increased risk of suffering from pancreatic cancer compared to a normal individual.

As used herein, the term subject or “test subject” indicates a mammal, in particular a human or non-human primate. The test subject may or may not be in need of an assessment of a predisposition to pancreatic cancer. For example, the test subject may have a condition or may have been exposed to conditions that are associated with pancreatic cancer prior to applying the methods described herein. In another example, the test subject has not been identified as a subject that may have a condition or may have been exposed to injuries or conditions that are associated with pancreatic cancer prior to applying the methods and apparatuses disclosed herein.

As used herein, the term “increased risk” is used to mean that the test subject has an increased chance of developing or acquiring pancreatic cancer compared to a normal individual. The increased risk may be relative or absolute and may be expressed qualitatively or quantitatively. For example, an increased risk may be expressed as simply determining the subject's proteomic profile and placing the patient in an “increased risk” category, based upon previous population studies. Alternatively, a numerical expression of the subject's increased risk may be determined based upon the proteomic profile. As used herein, examples of expressions of an increased risk include but are not limited to, odds, probability, odds ratio, p-values, attributable risk, relative frequency, positive predictive value, negative predictive value, and relative risk.

In one example, the increased risk of a patient can be determined from p-values that are derived from association studies. Specifically, associations with specific profiles can be performed using regression analysis by regressing the proteomic profile with pancreatic cancer. In addition, the regression may or may not be corrected or adjusted for one or more factors. The factors for which the analyses may be adjusted include, but are not limited to age, sex, weight, ethnicity, geographic location, general health of the subject, alcohol or drug consumption, caffeine or nicotine intake and the subject's apolipoprotein E (ApoE) genotype.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring.

As used herein, the phrase “proteomic profile” means the combination of a subject's proteins found in the peripheral blood or portions thereof, such as but not limited to plasma or serum. The proteomic profile is a collection of measurements, such as but not limited to a quantity or concentration, for individual proteins taken from a test sample of the subject. Examples of test samples or sources of components for the proteomic profile include, but are not limited to, biological fluids, which can be tested by suitable methods described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

Techniques to assay levels of individual components of the proteomic profile from test samples are well known to the skilled technician, and the methods and apparatuses of this disclosure are is not limited by the means by which the components are assessed. In one example, levels of the individual components of the proteomic profile are assessed using mass spectrometry in conjunction with ultra-performance liquid chromatography (UPLC), high-performance liquid chromatography (HPLC), and UPLC to name a few. Other methods of assessing levels of the individual components include biological methods, such as but not limited to ELISA assays.

The assessment of the levels of the individual components of the proteomic profile can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the proteomic profile, a sample is taken from the subject. The sample may or may not processed prior assaying levels of the components of the proteomic profile. For example, whole blood may be taken from an individual and the blood sample may be processed, e.g., centrifuged, to isolate plasma or serum from the blood. The sample may or may not be stored, e.g., frozen, prior to processing or analysis.

In one example, the individual levels of each of the proteins are lower than those compared to normal levels. In another example, the individual levels of some of the proteins are lower than those compared to normal levels.

In other examples, the individual levels of each of the proteins are higher than those compared to normal levels. In another example, the individual levels of some of the proteins are higher than those compared to normal levels.

The levels of depletion or augmentation of the proteins compared to normal levels can vary. In one example, the levels of any one or more of the proteins is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55 or 60 times lower than normal levels. In another example, the levels of any one or more of the proteins is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55 or 60 times higher than normal levels. The number of “times” the levels of a protein is lower or higher over normal can be a relative or absolute number of times. In the alternative, the levels of the proteins may be normalized to a standard and these normalized levels can then be compared to one another to determine if a protein is lower or higher.

The subject's proteomic profile may be compared to the profile that is deemed to be a normal proteomic profile. The proteomic profile of an individual or group of individuals without pancreatic cancer can be used to establish a “normal proteomic profile.” In one example, a normal proteomic profile can be ascertained from the same subject having no signs (clinical or otherwise) of pancreatic cancer. In one example, a “normal” proteomic profile is assessed in the same subject from whom the sample is taken prior to the onset of pancreatic cancer. That is, the term “normal” with respect to a proteomic profile can be used to mean the subject's baseline proteomic profile prior to the onset of pancreatic cancer. The proteomic profile can then be reassessed periodically and compared to the subject's baseline proteomic profile.

Thus, the present disclosure also includes methods of monitoring the progression of pancreatic cancer in a subject, with the methods comprising determining the subject's proteomic profile more than once over a period of time. For example, some examples may include determining the subject's proteomic profile two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having pancreatic cancer would also include examples in which the subject's proteomic profile is assessed during and after treatment of pancreatic cancer. In other words, also disclosed are includes methods of monitoring the efficacy of treatment of proteomic impairment by assessing the subject's proteomic profile over the course of the treatment and after the treatment.

In another example, a normal proteomic profile is assessed in a sample from a different subject or patient (from the subject being analyzed) and this different subject does not have or is not suspected of having pancreatic cancer. In still another example, the normal proteomic profile is assessed in a population of healthy individuals, the constituents of which display no pancreatic cancer. Thus, the subject's proteomic profile can be compared to a normal proteomic profile generated from a single normal sample or a proteomic profile generated from more than one normal sample.

Of course, measurements of the individual components, e.g., concentration, of the normal proteomic profile can fall within a range of values, and values that do not fall within this “normal range” are said to be outside the normal range. These measurements may or may not be converted to a value, number, factor or score as compared to measurements in the “normal range.” For example, a measurement for a specific protein that is below the normal range, may be assigned a value or −1, −2, −3, etc., depending on the scoring system devised.

In one example, the “proteomic profile value” can be a single value, number, factor or score given as an overall collective value to the individual molecular components of the profile. For example, if each component is assigned a value, such as above, the proteomic value may simply be the overall score of each individual value. For example, if 10 of the components of the proteomic profile are used to create the profile value, and five of the components are assigned values of“−2” and five are assigned values of“−1,” the proteomic profile in this example would be −15, with a normal value being, for example, “0.” In this manner, the proteomic profile value could be useful single number or score, the actual value or magnitude of which could be an indication of the actual risk of pancreatic cancer, e.g., the “more negative” or the “more positive” the value, the greater the risk of pancreatic cancer.

In another example the “proteomic profile value” can be a series of values, numbers, factors or scores given to the individual components of the overall profile. In another example, the “proteomic profile value” may be a combination of values, numbers, factors or scores given to individual components of the profile as well as values, numbers, factors or scores collectively given to a group of components. In another example, the proteomic profile value may comprise or consist of individual values, number, factors or scores for specific component as well as values, numbers, factors or scores for a group on components.

In another example, individual values from the proteins can be used to develop a single score, such as a “combined proteomic index,” which may utilize weighted scores from the individual component values reduced to a diagnostic number value. The combined proteomic index may also be generated using non-weighted scores from the individual component values. When the “combined proteomic index” exceeds (or is less than) a specific threshold level, the individual has a high risk of pancreatic cancer, whereas the maintaining a normal range value of the “combined proteomic index” would indicate a low or minimal risk of pancreatic cancer. In this example, the threshold value would be set by the combined proteomic index from normal subjects.

In another example, the value of the proteomic profile can be the collection of data from the individual measurements and need not be converted to a scoring system, such that the “proteomic profile value” is a collection of the individual measurements of the individual components of the profile.

If it is determined that a subject has an increased risk of pancreatic cancer, the attending health care provider may subsequently prescribe or institute a treatment program. Therefore, methods of screening individuals as candidates for treatment of pancreatic cancer are also provided herein. The attending healthcare worker may begin treatment, based on the subject's proteomic profile, before there are perceivable, noticeable or measurable signs of pancreatic cancer in the individual.

Similarly, methods disclosed herein may also be of use for monitoring the effectiveness of a treatment for pancreatic cancer. Once a treatment regimen has been established, with or without the use of the methods and apparatuses disclosed herein, to assist in a diagnosis of pancreatic cancer, the methods of monitoring a subject's proteomic profile over time can be used to assess the effectiveness of a pancreatic cancer treatment. Specifically, the subject's proteomic profile can be assessed over time, including before, during and after treatments for pancreatic cancer. The proteomic profile can be monitored, with, for example, a decline or an increase in the values of the profile over time being indicative that the treatment may or may not be as effective as desired.

### Quantitative Proteomics Identifies Brain Acid Soluble Protein 1 (BASP1) as a Prognostic Biomarker Candidate in Pancreatic Cancer Tissue

The study described herein provides an example of a method for identifying a prognostic biomarker for use in diagnosing pancreatic cancer, however, one of skill in the art will understand that such a method may be applicable to all manner of disease states. In this example, global protein sequencing of fresh frozen pancreatic cancer and healthy pancreas tissue samples was conducted by MS to discover potential protein biomarkers. Selected candidate proteins were further verified by targeted proteomics using parallel reaction monitoring (PRM). The expression of biomarker candidates was validated by immunohistochemistry in a large tissue microarray (TMA) cohort of 141 patients with resectable pancreatic cancer. Kaplan-Meier and Cox proportional hazard modelling was used to investigate the prognostic utility of candidate protein markers.

In the initial MS-discovery phase, 165 proteins were identified as potential biomarkers. In the subsequent MS-verification phase, a panel of 45 candidate proteins was verified by the development of a PRM assay. We found brain acid soluble protein 1 (BASP1) to be significantly upregulated in pancreatic cancer and have identified it as a new biomarker for pancreatic cancer possessing largely unknown biological and clinical functions and selected this marker for further analysis. We conducted external validation by tissue microarray (TMA) and immunohistochemistry in a large cohort showed that BASP1 overexpression significantly correlated to survival and response to chemotherapy in patients with pancreatic cancer.

Bioinformatic analysis and pathway analysis linked to clinical data indicated that BASP1 interacts with Wilms tumor protein (WT1) in pancreatic cancer. TMA-based immunohistochemistry analysis showed that BASP1 was an independent predictor of prolonged survival (HR 0.468, 95% CI 0.257-0.852, p=0.013) and predicted favorable response to adjuvant chemotherapy, whereas WT1 indicated a worsened survival (HR 1.636, 95% CI 1.083-2.473, p=0.019) and resistance to chemotherapy. Interaction analysis showed that patients with negative BASP1 and high WT1 expression had the poorest outcome (HR 3.536, 95% CI 1.336-9.362, p=0.011). Bioinformatic analysis and clinical data from our study provides a basis for using BASP1 and its putative interaction partner WT1 as biomarkers for predicting outcomes in pancreatic cancer patients.

### Study Design

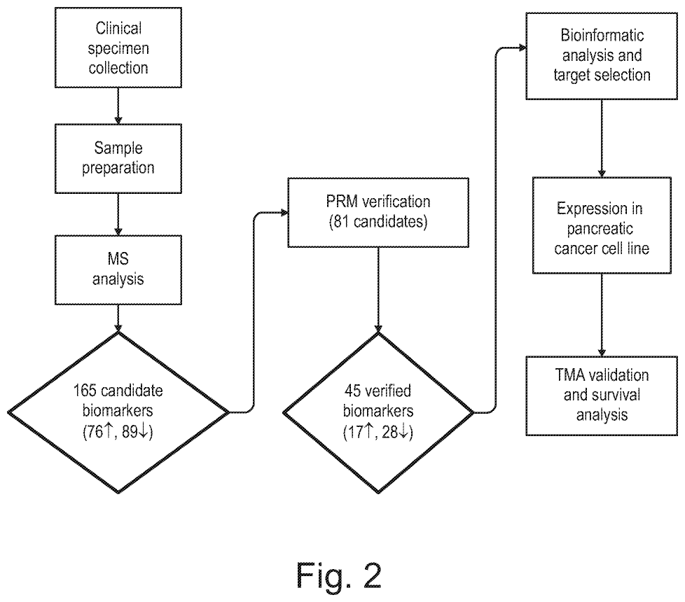

The methodological workflow of the present study is illustrated in FIG. 2. A Nano-liquid chromatography-tandem mass spectrometry (LC-MS/MS) platform was used for identification of candidate protein biomarkers for pancreatic cancer. Parallel Reaction Monitoring (PRM) was used for verification of protein biomarker candidates. Comprehensive bioinformatics analyses of candidate proteins and biological interaction partners were conducted to characterize functional relevance. Antibody-based validation was performed in a pancreatic cancer cell line and resected pancreatic cancer tissues from a larger cohort (Table 3). Protein expression levels were then integrated with clinicopathological information for survival analyses.

### Patients and Tissue Samples

For MS analysis, fresh frozen pancreatic cancer tissue samples (n=10 for MS discovery, n=8 for targeted MS) were prospectively collected from patients undergoing pancreaticoduodenectomy due to tumors located in the head of the pancreas between July 2013 and April 2015 at the Department of Surgery, Slane University Hospital, Lund, Sweden. Age and gender-matched fresh frozen normal pancreas (n=10) from organ donors free of any pancreatic disease were obtained from Lund University Diabetes Center and used as healthy controls (HC). Written informed consent was obtained from participating patients. For tissue microarray (TMA) and immunohistochemistry (IHC) analysis, FFPE tissue samples (n=143) were included from a retrospective cohort of pancreatic cancer patients who underwent surgery with curative intent from 1995 to 2017 at Slane University Hospital in Lund and Malmo, Sweden. Following antibody optimization and staining, biomarker expression could be evaluated in 141 of the 143 (98.6%) of tumor samples included in the TMA. All samples were re-evaluated by a pancreatic pathologist to confirm the diagnosis and uniformity of staging. The REMARK guidelines were followed where applicable (McShane L M, Altman D G, Sauerbrei W, Taube S E, Gion M, Clark G M. Statistics Subcommittee of the NCIEWGoCD: REporting recommendations for tumour MARKer prognostic studies (REMARK). Br J Cancer 2005; 93(4):387-91).

### MS Studies

### Tissue Sample Preparation

Individual fresh frozen tissue samples were pulverized in liquid N2 and thoroughly homogenized in an extraction buffer consisting of 500 mM Tris-C1 [pH 8], 6 M guanidine-HCl in 50 mM ammonium bicarbonate (AMBIC) along with protease and phosphatase inhibitor cocktail. The obtained extracts were then subjected to 4 freeze-thaw cycles, followed by ultrasonic bath for 20 min at 0° C. The soluble proteins were then reduced with 15 mM dithiothreitol (DTT) for 60 min at 60° C., alkylated using 50 mM iodoacetamide (IAA) for 30 min at room temperature in the dark, precipitated with a sample to ethanol (99.5%) ratio of 1:9 at −20° C. The protein precipitates were dissolved in 50 mM AMBIC and digested at 37° C. overnight using Mass Spec Grade Trypsin/Lys-C Mix (Promega, Madison, Wis., USA), with an enzyme to protein ratio of 1:100. The digested samples were dried and dissolved in 50 μl 0.1% Formic Acid (mobile phase A), and the concentration was specified using Pierce quantitative colorimetric peptide assay from Thermo Scientific (Rockford, Ill., USA). Finally, to enable normalization and as a control of the chromatographic performance, 25 fmol peptide retention time mixture (PRTC) (Thermo Fisher) consisting of 15 peptides was added to each sample.

### LC-MS/MS Analysis

The analytical platform, including a high-performance nanoflow liquid chromatography (HPLC) system (EASY-nLCTM™ 1000) and a Plus Hybrid Quadrupole-Orbitrap mass spectrometer (Q Exactive™) equipped with a nanospray ion source (EASY-Spray™), were manufactured by Thermo Fisher Scientific (Bremen, Germany). Individual samples containing 1 μg of peptide mixture in mobile phase A were injected at a flowrate of 300 nl min-1, separated by a 132 min gradient of 5-22% acetonitrile (ACN) in mobile phase A, followed by an 18 min gradient of 22-38% ACN in mobile phase A. Subsequent separation was conducted by a two-column system including the EASY-Spray analytical column (25 cm×75 μm ID, particle size 2 μm, pore size 100 Å, PepMap C18) tandem with the Acclaim pre-column (2 cm×75 μm ID, particle size 3 μm, pore size 100 Å, PepMap C18). The Orbitrap system was operated in the positive data-dependent acquisition (DDA) mode with an automatic switch between the full scanMS and MS/MS acquisition. On the precursors with the highest intensity, 15 data-dependent higher energy collision dissociation MS/MS scans were implemented. For the peptide detection, a full MS survey scan was performed in the Orbitrap detector. The MS scans with a resolution of 70,000 at 200 m/z, recording window between 400.0 and 1600.0 m/z, and automatic gain control (AGC) target value of 1×10\hat{ }6 with a maximum injection time of 100 ms. The resolution of the data dependent MS/MS scans was fixed of 17,500 at 200 m/z, values for the AGC target of 5×10\hat{ }5 and maximum injection time was 80 ms. The normalized collision energy was set on 27.0% for all scans.

### Targeted Proteomics Analysis

PRM analysis was performed to verify differentially expressed proteins.

One or 2 unique peptides of each targeted protein were selected from the discovery measurements, depending on detection frequencies >50%, missed cleavage=0 and p-value <0.05, along with peptide intensities and ranking of peptide spectrum matches. Finally, a spectral library of 81 selected proteins (from the 165 differentially expressed proteins as well as the proteins only detectable in one condition) including 150 peptides was created. Owing to inadequate tissue sample volume, we had to exclude 2 pancreatic cancer subjects from the PRM phase. The proteins extracted from 18 fresh frozen samples (8 pancreatic cancer samples vs. 10 healthy controls) were reduced, alkylated, and digested as described previously in sample preparation. One microgram of the sample was injected into the LC-MS/MS system, and the PRM assay was set in a time-scheduled acquisition mode with a retention time+/−5 min and resolution at 35000 (AGC target to 5×10\hat{ }5, maximum injection time of 50 ms). The chromatographic peak width was 30 s, normalized collision energy on 26.0%, and the isolation window of 2 m/z. Skyline software was used for relative quantification in the PRM study (Henderson C M, et al. Clin Chem 2018; 64(2):408-10).

### MS Data Analysis

Each sample was measured in duplicate by LC-MS/MS in a randomized order. The raw files generated from the duplicates were combined and evaluated using Proteome Discoverer software (Thermo Fisher) Version 1.4 focusing on high confidence peptides only. The spectra selection settings: minimum and maximum precursor mass at 350 Da and 5000 Da, respectively; signal-to-noise (s/n) threshold 1.5. Parameters for SEQUEST HT (Tabb D L. The SEQUEST family tree. J Am Soc Mass Spectrom 2015; 26(11):1814-9) were set as follows: precursor mass tolerance of 10 ppm (p.p.m); fragment mass tolerance of 0.02 Da; trypsin as the enzyme; one missed cleavage site was accepted. Based on the UniProtKB human database (Chen C, et al. Methods Mol Biol 2017; 1558:3-39), dynamic modifications were included, such as: methyl (+14.016 Da; K, R), dimethyl (+28.031 Da; K, R), acetyl (+42.011 Da; K), trimethyl (+42.047 Da; K, R), glygly (+114.043 Da; K), oxidation (+15.995 Da; M), and the fixed modification carbamidomethyl (+57.021 Da; C). The percolator was applied for the processing node, and the false discovery rate (FDR) value was set to 0.01. To quantify the peptides, the precursor ions area detector was used in the search engine (Proteome discoverer; Thermo Scientific), protein groups identified>2 peptides from all samples were considered for further analysis and only unique peptides were used for protein quantification.

### Tissue Microarray Construction and Immunohistochemistry

Archival FFPE pancreatic cancer specimens from the larger validation cohort were subjected to TMA. Employing an automated tissue array instrument (Minicore®3, Alphelys, Plaisir, France), 4 cores of cancer tissue from each specimen (diameter at 2 mm, selected by a pathologist) were extracted and fixed into paraffin blocks. After quality control, the TMA blocks were sectioned into 3 μm thick slides for IHC analysis.

IHC was performed as described previously (Hu D, Ansari D, Zhou Q, Sasor A, Hilmersson K S, Bauden M, et al. Calcium-activated chloride channel regulator 1 as a prognostic biomarker in pancreatic ductal adenocarcinoma. BMC Cancer 2018; 18(1):1096). Briefly, after de-paraffinization, rehydration and antigen-retrieval, TMA-slides were incubated with primary antibodies (rabbit anti-human BASP1 (dilution 1:100; Cat No. HPA045218, Atlas Antibodies); mouse anti-humanWT1 (clone 6F-H2, Ready-to-Use, Cat No. IS05530-2, DAKO)) overnight at 4° C. Next, slides were incubated with second antibody (for BASP1, biotinylated goat anti-rabbit (dilution 1:200; Cat No. BA-1000, Vector Laboratories, Burlingame, Calif.); for WT1, biotinylated horse anti-mouse (dilution 1:200, Vector Laboratories, Cat No. BA-2000)) followed by staining with avidin-biotin-peroxidase complex (Vectastain Elite ABCHRP Kit, Cat No. PK-6100, Vector Laboratories, Burlingame, Calif.). The sections were then incubated with chromogen diaminobenzidine (DAB) (Cat No. SK-4100, Vector Laboratories, Burlingame, Calif.) and counter stained with haematoxylin and mounted with xylene based medium. The IHC scoring was performed by an experienced pancreas pathologist (A.S.) who was blinded to the clinical information. Scoring was based on the percentage of positive tumor cells and the staining intensity. IHC results were scored as follows: 0=negative; 1=weak; 2=moderate; and 3=strong. For tumors that showed heterogeneous staining, the predominant pattern was taken into account for scoring.

### Cell Culture and Immunofluorescence

The human pancreatic cancer cell line, PANC-1, was purchased from ATCC-LGC Standards (Manassas, Va., USA). The cells were maintained in Dulbecco's modified Eagle's medium (DMEM; Life Technologies, CA, USA) supplemented with 10% fetal bovine serum and antibiotics (100 U/ml penicillin and 100 μg/ml streptomycin) in a humidified 5% CO2 atmosphere at 37° C.

For investigating intracellular localization, PANC-1 cells were cultured (8×10\hat{ }3 cells/well) in eight-well chamber slides (Lab-Tek II Chamber Slide System, Nunc). After 48 h, the cells were fixed with 4% formaldehyde, then permeabilized with 1% Triton X-100, blocked with 5% goat serum and incubated with mouse anti-human WT1 (clone 6FH2, Ready-to-Use; Cat No. IS05530-2, DAKO) at room temperature for 2 h. After washing, cells were moved into dark environment, Goat anti-Mouse Alexa Fluor 594 (dilution 1:500; Cat No. A11032, Invitrogen) was added at room temperature for 1 h. Subsequently, the cells were blocked with 5% donkey serum and incubated with rabbit anti-human BASP1 (dilution 1:50; Cat No. HPA045218, Atlas Antibodies) at room temperature for 2 h. Following washing, Donkey-anti-Rabbit Alexa Fluor 488 (dilution 1:500; Cat No. A21206, Invitrogen) was added at room temperature for 1 h. Finally, the cells were incubated with DAPI to stain the nuclei. Positive staining was visualized using a Nikon Eclipse 80i microscope with a Nikon DS-Qi1 camera and analyzed using NIS-Elements software (Nikon Instruments Inc.; Melville, N.Y., USA).

### Statistics and Bioinformatics

Perseus software (Tyanova S, Temu T, Sinitcyn P, Carlson A, Hein M Y, Geiger T, et al. The Perseus computational platform for comprehensive analysis of (prote)omics data. Nat Methods 2016; 13(9):731-40) version 1.6.0.7 was used for the statistical analysis of the MS results. The protein intensities were log 2 transformed and normalized by subtracting the median intensity of all the proteins per sample. Replacing the missing values from a normal distribution was performed though data imputation by using the following settings: width 0.3 and downshift 0. A Two-Sample Student's t-test (two-tailed) followed by permutation-based FDR correction was performed to compare protein levels between the groups. The settings included S0=2, which is a parameter used to calculate the relative difference (ratio of change in protein expression to standard deviation) between group means. It defines the within groups variance, the relative importance of the resulted p-values, and the difference between means of log 2 intensities (Tusher V G, et al. Proc Natl Acad Sci 2001; 98(9):5116-21). Finally, the proteins with FDR adjusted p-value (or q-value) of 0.01 were considered as differentially expressed.

For bioinformatic analysis of networks involving the biological relationship between BASP1 and WT1, the Ingenuity Pathway Analysis software (IPA, Qiagen, Inc. Redwood City, Calif., USA) was used. This toolset builds upon a literature-derived relationship knowledge base. A network involving all direct interactors of these proteins was built and analyzed for pathway enrichment and functional annotations. Additionally, differentially expressed proteins between pancreatic cancer and healthy controls samples from MS discovery were mapped onto the BASP1/WT1 network. Subcellular localization of significantly up- and down-regulated proteins in pancreatic cancer versus healthy control samples was manually assessed using UniProt (On the World-Wide Web at uniprot.org/). PANTHER (Mi H, et al. Nucleic Acids Res 2010; 38(suppl_1): D204-10), also on the World-Wide Web at pantherdb.org/) was employed to identify gene ontology terms of the significantly differentially expressed proteins.

For IHC analysis, the correlation between the expression levels of protein biomarkers and clinicopathological parameters was estimated using the Mann-Whitney U test for continuous variables and Fisher's exact test or χ2 for categorical variables. Kaplan-Meier analysis was used to calculate the cumulative probability of overall survival (OS), log-rank tests were used to evaluate the differences. Prognostic factors were calculated using univariable and multivariable analysis (Cox proportional hazards regression model). A value of p<0.05 was considered statistically significant.

Statistical evaluation was conducted with Perseus software version 1.6.0.7, SPSS version 23.0 (SPSS Inc., Chicago, Ill., USA), GraphPad Prism v.7 (La Jolla, Calif., USA), and R (Team R C. R: A language and environment for statistical computing; 2013) programming language version 3.5.1 (R Foundation for Statistical Computing, on the World-Wide Web at: r-project.org/).

### Results

### Identification of Candidate Biomarkers for Pancreatic Cancer

Representative fresh frozen pancreatic cancer (n=10) and healthy control (n=10) tissue samples were analyzed using a LC-MS/MS platform. A total of 4138 proteins were identified and 2950 proteins were quantified with one or more unique peptides (see Table A).

TABLE A

| | UniProt | | |
|---|---|---|---|
| No. | Accession | Gene | Protein name |
| 1 | A0MZ66 | SHOT1 | Shootin-1 |
| 2 | A1L4H1 | SRCRL | Soluble scavenger receptor cysteine-rich domain-containing protein |
| | | | SSC5D |
| 3 | A5A3E0 | POTEF | POTE ankyrin domain family member F |
| 4 | A6NC98 | CC88B | Coiled-coil domain-containing protein 88B |
| 5 | A6NDB9 | PALM3 | Paralemmin-3 |
| 6 | A6NE01 | F186A | Protein FAM186A |
| 7 | A6NFE2 | SMCO2 | Single-pass membrane and coiled-coil domain-containing protein 2 |
| 8 | A6NI28 | RHG42 | Rho GTPase-activating protein 42 |
| 9 | A6NMK8 | F196B | Protein FAM196B |
| 10 | A6NNC1 | P12LL | Putative POM121-like protein 1-like |
| 11 | C9J6K1 | CS081 | Putative uncharacterized protein C19orf81 |
| 12 | C9JLW8 | F195B | Protein FAM195B |
| 13 | O00115 | DNS2A | Deoxyribonuclease-2-alpha |
| 14 | O00151 | PDLI1 | PDZ and LIM domain protein 1 |
| 15 | O00159 | MYO1C | Unconventional myosin-lc |
| 16 | O00161 | SNP23 | Synaptosomal-associated protein 23 |
| 17 | O00192 | ARVC | Armadillo repeat protein deleted in velo-cardio-facial syndrome |
| 18 | O00193 | SMAP | Small acidic protein |
| 19 | O00217 | NDUS8 | NADH dehydrogenase [ubiquinone] iron-sulfur protein 8, mitochondrial |
| 20 | O00233 | PSMD9 | 26S proteasome non-ATPase regulatory subunit 9 |
| 21 | O00244 | ATOX1 | Copper transport protein ATOX1 |
| 22 | O00264 | PGRC1 | Membrane-associated progesterone receptor component 1 |
| 23 | O00267 | SPT5H | Transcription elongation factor SPT5 |
| 24 | O00273 | DFFA | DNA fragmentation factor subunit alpha |
| 25 | O00291 | HIP1 | Huntingtin-interacting protein 1 |
| 26 | O00299 | CLIC1 | Chloride intracellular channel protein 1 |
| 27 | O00303 | EIF3F | Eukaryotic translation initiation factor 3 subunit F |
| 28 | O00330 | ODPX | Pyruvate dehydrogenase protein X component, mitochondrial |
| 29 | O00399 | DCTN6 | Dynactin subunit 6 |
| 30 | O00401 | WASL | Neural Wiskott-Aldrich syndrome protein |
| 31 | O00422 | SAP18 | Histone deacetylase complex subunit SAP18 |
| 32 | O00442 | RTCA | RNA 3′-terminal phosphate cyclase |
| 33 | O00468 | AGRIN | Agrin; Agrin C-terminal 110 kDa subunit; Agrin C-terminal 22 kDa fragment |
| 34 | O00483 | NDUA4 | Cytochrome c oxidase subunit NDUFA4 |
| 35 | O00499 | BIN1 | Myc box-dependent-interacting protein 1 |
| 36 | O00505 | IMA4 | Importin subunit alpha-4 |
| 37 | O00515 | LAD1 | Ladinin-1 |
| 38 | O00567 | NOP56 | Nucleolar protein 56 |
| 39 | O00571 | DDX3X | ATP-dependent RNA helicase DDX3X |
| 40 | O00629 | IMA3 | Importin subunit alpha-3 |
| 41 | O00757 | F16P2 | Fructose-1,6-bisphosphatase isozyme 2 |
| 42 | O00764 | PDXK | Pyridoxal kinase |
| 43 | O14545 | TRAD1 | TRAF-type zinc finger domain-containing protein 1 |
| 44 | O14558 | HSPB6 | Heat shock protein beta-6 |
| 45 | O14561 | ACPM | Acyl carrier protein, mitochondrial |
| 46 | O14562 | UBFD1 | Ubiquitin domain-containing protein UBFD1 |
| 47 | O14579 | COPE | Coatomer subunit epsilon |
| 48 | O14617 | AP3D1 | AP-3 complex subunit delta-1 |
| 49 | O14618 | CCS | Copper chaperone for superoxide dismutase |
| 50 | O14639 | ABLM1 | Actin-binding LIM protein 1 |
| 51 | O14653 | GOSR2 | Golgi SNAP receptor complex member 2 |
| 52 | O14737 | PDCD5 | Programmed cell death protein 5 |
| 53 | O14745 | NHRF1 | Na(+)/H(+) exchange regulatory cofactor NHE-RF1 |
| 54 | O14773 | TPP1 | Tripeptidyl-peptidase 1 |
| 55 | O14776 | TCRG1 | Transcription elongation regulator 1 |
| 56 | O14818 | PSA7 | Proteasome subunit alpha type-7 |
| 57 | O14907 | TX1B3 | Tax1-binding protein 3 |
| 58 | O14950 | ML12B | Myosin regulatory light chain 12B |
| 59 | O14964 | HGS | Hepatocyte growth factor-regulated tyrosine kinase substrate |
| 60 | O14974 | MYPT1 | Protein phosphatase 1 regulatory subunit 12A |
| 61 | O14976 | GAK | Cydin-G-associated kinase |
| 62 | O14979 | HNRDL | Heterogeneous nuclear ribonucleoprotein D-like |
| 63 | O15020 | SPTN2 | Spectrin beta chain, non-erythrocytic 2 |
| 64 | O15027 | SC16A | Protein transport protein Sec16A |
| 65 | O15031 | PLXB2 | Plexin-B2 |
| 66 | O15061 | SYNEM | Synemin |
| 67 | O15083 | ERC2 | ERC protein 2 |
| 68 | O15143 | ARC1B | Actin-related protein 2/3 complex subunit 1B |
| 69 | O15144 | ARPC2 | Actin-related protein 2/3 complex subunit 2 |
| 70 | O15145 | ARPC3 | Actin-related protein 2/3 complex subunit 3 |
| 71 | O15173 | PGRC2 | Membrane-associated progesterone receptor component 2 |
| 72 | O15212 | PFD6 | Prefoldin subunit 6 |
| 73 | O15230 | LAMA5 | Laminin subunit alpha-5 |
| 74 | O15231 | ZN185 | Zinc finger protein 185 |
| 75 | O15235 | RT12 | 28S ribosomal protein S12, mitochondrial |
| 76 | O15240 | VGF | Antimicrobial peptide VGF[554-577]; Neuroendocrine regulatory |
| | | | peptide-1 |
| 77 | O15305 | PMM2 | Phosphomannomutase 2 |
| 78 | O15320 | CTGE5 | cTAGE family member 5 |
| 79 | O15347 | HMGB3 | High mobility group protein B3 |
| 80 | O15371 | EIF3D | Eukaryotic translation initiation factor 3 subunit D |
| 81 | O15372 | EIF3H | Eukaryotic translation initiation factor 3 subunit H |
| 82 | O15382 | BCAT2 | Branched-chain-amino-acid aminotransferase, mitochondrial |
| 83 | O15400 | STX7 | Syntaxin-7 |
| 84 | O15417 | TNC18 | Trinucleotide repeat-containing gene 18 protein |
| 85 | O15511 | ARPC5 | Actin-related protein 2/3 complex subunit 5 |
| 86 | O15523 | DDX3Y | ATP-dependent RNA helicase DDX3Y |
| 87 | O15541 | R113A | RING finger protein 113A |
| 88 | O43172 | PRP4 | U4/U6 small nuclear ribonucleoprotein Prp4 |
| 89 | O43175 | SERA | D-3-phosphoglycerate dehydrogenase |
| 90 | O43181 | NDUS4 | NADH dehydrogenase [ubiquinone] iron-sulfur protein 4, mitochondrial |
| 91 | O43237 | DC1L2 | Cytoplasmic dynein 1 light intermediate chain 2 |
| 92 | O43242 | PSMD3 | 26S proteasome non-ATPase regulatory subunit 3 |
| 93 | O43290 | SNUT1 | U4/U6.U5 tri-snRNP-associated protein 1 |
| 94 | O43324 | MCA3 | Eukaryotic translation elongation factor 1 epsilon-1 |
| 95 | O43390 | HNRPR | Heterogeneous nuclear ribonucleoprotein R |
| 96 | O43396 | TXNL1 | Thioredoxin-like protein 1 |
| 97 | O43399 | TPD54 | Tumor protein D54 |
| 98 | O43491 | E41L2 | Band 4.1-like protein 2 |
| 99 | O43493 | TGON2 | Trans-Golgi network integral membrane protein 2 |
| 100 | O43504 | LTOR5 | Ragulator complex protein LAMTOR5 |
| 101 | O43524 | FOXO3 | Forkhead box protein 03 |
| 102 | O43570 | CAH12 | Carbonic anhydrase 12 |
| 103 | O43583 | DENR | Density-regulated protein |
| 104 | O43598 | DNPH1 | 2′-deoxynucleoside 5′-phosphate N-hydrolase 1 |
| 105 | O43615 | TIM44 | Mitochondrial import inner membrane translocase subunit TIM44 |
| 106 | O43633 | CHM2A | Charged multi vesicular body protein 2a |
| 107 | O43639 | NCK2 | Cytoplasmic protein NCK2 |
| 108 | O43670 | ZN207 | BUB3-interacting and GLEBS motif-containing protein ZNF207 |
| 109 | O43678 | NDUA2 | NADH dehydrogenase [ubiquinone] 1 alpha subcomplex subunit 2 |
| 110 | O43684 | BUB3 | Mitotic checkpoint protein BUB3 |
| 111 | O43707 | ACTN4 | Alpha-actinin-4 |
| 112 | O43715 | TRIA1 | TP53-regulated inhibitor of apoptosis 1 |
| 113 | O43765 | SGTA | Small glutamine-rich tetratricopeptide repeat-containing protein alpha |
| 114 | O43768 | ENSA | Alpha-endosulfine |
| 115 | O43776 | SYNC | Asparagine-tRNA ligase, cytoplasmic |
| 116 | O43813 | LANC1 | LanC-like protein 1 |
| 117 | O43823 | AKAP8 | A-kinase anchor protein 8 |
| 118 | O43837 | IDH3B | Isocitrate dehydrogenase [NAD] subunit beta, mitochondrial |
| 119 | O43852 | CALU | Calumenin |
| 120 | O43865 | SAHH2 | Putative adenosyl homocysteinase 2 |
| 121 | O43916 | CHST1 | Carbohydrate sulfotransferase 1 |
| 122 | O43920 | NDUS5 | NADH dehydrogenase [ubiquinone] iron-sulfur protein 5 |
| 123 | O60220 | TIM8A | Mitochondrial import inner membrane translocase subunit Tim8 A |
| 124 | O60232 | SSA27 | Sjoegren syndrome/scleroderma autoantigen 1 |
| 125 | O60237 | MYPT2 | Protein phosphatase 1 regulatory subunit 12B |
| 126 | O60240 | PLIN1 | Perilipin-1 |
| 127 | O60271 | JIP4 | C-Jun-amino-terminal kinase-interacting protein 4 |
| 128 | O60282 | KIF5C | Kinesin heavy chain isoform 5C |
| 129 | O60313 | OPA1 | Dynamin-like 120 kDa protein, form S1; Dynamin-like 120 kDa protein, |
| | | | mitochondrial |
| 130 | O60315 | ZEB2 | Zinc finger E-box-binding homeobox 2 |
| 131 | O60356 | NUPR1 | Nuclear protein 1 |
| 132 | O60437 | PEPL | Periplakin |
| 133 | O60493 | SNX3 | Sorting nexin-3 |
| 134 | O60504 | VINEX | Vinexin |
| 135 | O60506 | HNRPQ | Heterogeneous nuclear ribonucleoprotein Q |
| 136 | O60547 | GMDS | GDP-mannose 4,6 dehydratase |
| 137 | O60565 | GREM1 | Gremlin-1 |
| 138 | O60610 | DIAP1 | Protein diaphanous homolog 1 |
| 139 | O60613 | 42248 | 15 kDa selenoprotein |
| 140 | O60664 | PLIN3 | Perilipin-3 |
| 141 | O60701 | UGDH | UDP-glucose 6-dehydrogenase |
| 142 | O60704 | TPST2 | Protein-tyrosine sulfotransferase 2 |
| 143 | O60716 | CTND1 | Catenin delta-1 |
| 144 | O60739 | EIF1B | Eukaryotic translation initiation factor 1b |
| 145 | O60749 | SNX2 | Sorting nexin-2 |
| 146 | O60763 | USO1 | General vesicular transport factor p115 |
| 147 | O60784 | TOM1 | Target of Myb protein 1 |
| 148 | O60828 | PQBP1 | Polyglutamine-binding protein 1 |
| 149 | O60832 | DKC1 | H/ACA ribonucleoprotein complex subunit 4 |
| 150 | O60841 | IF2P | Eukaryotic translation initiation factor 5B |
| 151 | O60869 | EDF1 | Endothelial differentiation-related factor 1 |
| 152 | O60884 | DNJA2 | DnaJ homolog subfamily A member 2 |
| 153 | O60888 | CUTA | Protein CutA |
| 154 | O60925 | PFD1 | Prefoldin subunit 1 |
| 155 | O60927 | PP1RB | Protein phosphatase 1 regulatory subunit 11 |
| 156 | O60936 | NOL3 | Nucleolar protein 3 |
| 157 | O75083 | WDR1 | WD repeat-containing protein 1 |
| 158 | O75112 | LDB3 | LIM domain-binding protein 3 |
| 159 | O75128 | COBL | Protein cordon-bleu |
| 160 | O75146 | HIP1R | Huntingtin-interacting protein 1-related protein |
| 161 | O75150 | BRE1B | E3 ubiquitin-protein ligase BRE1B |
| 162 | O75152 | ZC11A | Zinc finger CCCH domain-containing protein 11A |
| 163 | O75157 | T22D2 | TSC22 domain family protein 2 |
| 164 | O75175 | CNOT3 | CCR4-NOT transcription complex subunit 3 |
| 165 | O75208 | COQ9 | Ubiquinone biosynthesis protein COQ9, mitochondrial |
| 166 | O75223 | GGCT | Gamma-glutamylcydotransferase |
| 167 | O75347 | TBCA | Tubulin-specific chaperone A |
| 168 | O75348 | VATG1 | V-type proton ATPase subunit G 1 |
| 169 | O75351 | VPS4B | Vacuolar protein sorting-associated protein 4B |
| 170 | O75367 | H2AY | Core histone macro-H2A.1 |
| 171 | O75368 | SH3L1 | SH3 domain-binding glutamic acid-rich-like protein |
| 172 | O75369 | FLNB | Filamin-B |
| 173 | O75376 | NCOR1 | Nuclear receptor corepressor 1 |
| 174 | O75380 | NDUS6 | NADH dehydrogenase [ubiquinone] iron-sulfur protein 6, mitochondrial |
| 175 | O75390 | CISY | Citrate synthase, mitochondrial |
| 176 | O75391 | SPAG7 | Sperm-associated antigen 7 |
| 177 | O75396 | SC22B | Vesicle-trafficking protein SEC22b |
| 178 | O75400 | PR40A | Pre-mRNA-processing factor 40 homolog A |
| 179 | O75410 | TACC1 | Transforming acidic coiled-coil-containing protein 1 |
| 180 | O75475 | PSIP1 | PC4 and SFRS1-interacting protein |
| 181 | O75477 | ERLN1 | Erlin-1 |
| 182 | O75489 | NDUS3 | NADH dehydrogenase [ubiquinone] iron-sulfur protein 3, mitochondrial |
| 183 | O75494 | SRS10 | Serine/arginine-rich splicing factor 10 |
| 184 | O75521 | ECI2 | Enoyl-CoA delta isomerase 2, mitochondrial |
| 185 | O75531 | BAF | Barrier-to-autointegration factor |
| 186 | O75533 | SF3B1 | Splicing factor 3B subunit 1 |
| 187 | O75534 | CSDE1 | Cold shock domain-containing protein E1 |
| 188 | O75569 | PRKRA | Interferon-inducible double-stranded RNA-dependent protein kinase |
| | | | activator A |
| 189 | O75594 | PGRP1 | Peptidoglycan recognition protein 1 |
| 190 | O75607 | NPM3 | Nucleoplasmin-3 |
| 191 | O75643 | U520 | U5 small nuclear ribonucleoprotein 200 kDa helicase |
| 192 | O75764 | TCEA3 | Transcription elongation factor A protein 3 |
| 193 | O75781 | PALM | Paralemmin-1 |
| 194 | O75821 | EIF3G | Eukaryotic translation initiation factor 3 subunit G |
| 195 | O75822 | EIF3J | Eukaryotic translation initiation factor 3 subunit J |
| 196 | O75830 | SPI2 | Serpin I2 |
| 197 | O75874 | IDHC | Isocitrate dehydrogenase [NADP] cytoplasmic |
| 198 | O75880 | SCO1 | Protein SCO1 homolog, mitochondrial |
| 199 | O75886 | STAM2 | Signal transducing adapter molecule 2 |
| 200 | O75891 | AL1L1 | Cytosolic 10-formyltetrahydrofolate dehydrogenase |
| 201 | O75935 | DCTN3 | Dynactin subunit 3 |
| 202 | O75937 | DNJC8 | DnaJ homolog subfamily C member 8 |
| 203 | O75940 | SPF30 | Survival of motor neuron-related-splicing factor 30 |
| 204 | O75947 | ATP5H | ATP synthase subunit d, mitochondrial |
| 205 | O75955 | FLOT1 | Flotillin-1 |
| 206 | O76003 | GLRX3 | Glutaredoxin-3 |
| 207 | O76024 | WFS1 | Wolframin |
| 208 | O76038 | SEGN | Secretagogin |
| 209 | O76041 | NEBL | Nebulette |
| 210 | O76070 | SYUG | Gamma-synudein |
| 211 | O76094 | SRP72 | Signal recognition particle subunit SRP72 |
| 212 | O94760 | DDAH1 | N(G),N(G)-dimethylarginine dimethylaminohydrolase 1 |
| 213 | O94763 | RMP | Unconventional prefoldin RPB5 interactor 1 |
| 214 | O94811 | TPPP | Tubulin polymerization-promoting protein |
| 215 | O94819 | KBTBB | Kelch repeat and BTB domain-containing protein 11 |
| 216 | O94832 | MYO1D | Unconventional myosin-Id |
| 217 | O94842 | TOX4 | TOX high mobility group box family member 4 |
| 218 | O94855 | SC24D | Protein transport protein Sec24D |
| 219 | O94874 | UFL1 | E3 UFM1-protein ligase 1 |
| 220 | O94875 | SRBS2 | Sorbin and SH3 domain-containing protein 2 |
| 221 | O94888 | UBXN7 | UBX domain-containing protein 7 |
| 222 | O94903 | PROSC | Proline synthase co-transcribed bacterial homolog protein |
| 223 | O94905 | ERLN2 | Erlin-2 |
| 224 | O94906 | PRP6 | Pre-mRNA-processing factor 6 |
| 225 | O94925 | GLSK | Glutaminase kidney isoform, mitochondrial |
| 226 | O94979 | SC31A | Protein transport protein Sec31A |
| 227 | O95081 | AGFG2 | Arf-GAP domain and FG repeat-containing protein 2 |
| 228 | O95154 | ARK73 | Aflatoxin B1 aldehyde reductase member 3 |
| 229 | O95180 | CAC1H | Voltage-dependent T-type calcium channel subunit alpha-1H |
| 230 | O95182 | NDUA7 | NADH dehydrogenase [ubiquinone] 1 alpha subcomplex subunit 7 |
| 231 | O95202 | LETM1 | LETM1 and EF-hand domain-containing protein 1, mitochondrial |
| 232 | O95218 | ZRAB2 | Zinc finger Ran-binding domain-containing protein 2 |
| 233 | O95248 | MTMR5 | Myotubularin-related protein 5 |
| 234 | O95292 | VAPB | Vesicle-associated membrane protein-associated protein B/C |
| 235 | O95295 | SNAPN | SNARE-associated protein Snapin |
| 236 | O95336 | 6PGL | 6-phosphogluconolactonase |
| 237 | O95359 | TACC2 | Transforming acidic coiled-coil-containing protein 2 |
| 238 | O95394 | AGM1 | Phosphoacetylglucosamine mutase |
| 239 | O95425 | SVIL | Supervillin |
| 240 | O95486 | SC24A | Protein transport protein Sec24A |
| 241 | O95487 | SC24B | Protein transport protein Sec24B |
| 242 | O95613 | PCNT | Pericentrin |
| 243 | O95670 | VATG2 | V-type proton ATPase subunit G 2 |
| 244 | O95721 | SNP29 | Synaptosomal-associated protein 29 |
| 245 | O95747 | OXSR1 | Serine/threonine-protein kinase OSR1 |
| 246 | O95757 | HS74L | Heat shock 70 kDa protein 4L |
| 247 | O95777 | LSM8 | U6 snRNA-associated Sm-like protein LSm8 |
| 248 | O95793 | STAU1 | Double-stranded RNA-binding protein Staufen homolog 1 |
| 249 | O95810 | SDPR | Serum deprivation-response protein |
| 250 | O95817 | BAG3 | BAG family molecular chaperone regulator 3 |
| 251 | O95831 | AIFM1 | Apoptosis-inducing factor 1, mitochondrial |
| 252 | O95834 | EMAL2 | Echinoderm microtubule-associated protein-like 2 |
| 253 | O95865 | DDAH2 | N(G),N(G)-dimethylarginine dimethylaminohydrolase 2 |
| 254 | O95881 | TXD12 | Thioredoxin domain-containing protein 12 |
| 255 | O95926 | SYF2 | Pre-mRNA-splicing factor SYF2 |
| 256 | O95994 | AGR2 | Anterior gradient protein 2 homolog |
| 257 | O96000 | NDUBA | NADH dehydrogenase [ubiquinone] 1 beta subcomplex subunit 10 |
| 258 | O96033 | MOC2A | Molybdopterin synthase sulfur carrier subunit |
| 259 | P00167 | CYB5 | Cytochrome b5 |
| 260 | P00325 | ADH1B | Alcohol dehydrogenase 1B |
| 261 | P00326 | ADH1G | Alcohol dehydrogenase 1C |
| 262 | P00338 | LDHA | L-lactate dehydrogenase A chain |
| 263 | P00352 | AL1A1 | Retinal dehydrogenase 1 |
| 264 | P00367 | DHE3 | Glutamate dehydrogenase 1, mitochondrial |
| 265 | P00387 | NB5R3 | NADH-cytochrome b5 reductase 3 |
| 266 | P00390 | GSHR | Glutathione reductase, mitochondrial |
| 267 | P00441 | SODC | Superoxide dismutase [Cu—Zn] |
| 268 | P00450 | CERU | Ceruloplasmin |
| 269 | P00492 | HPRT | Hypoxanthine-guanine phosphoribosyltransferase |
| 270 | P00505 | AATM | Aspartate aminotransferase, mitochondrial |
| 271 | P00558 | PGK1 | Phosphoglycerate kinase 1 |
| 272 | P00568 | KAD1 | Adenylate kinase isoenzyme 1 |
| 273 | P00734 | THRB | Activation peptide fragment 1; Activation peptide fragment 2 |
| 274 | P00738 | HPT | Haptoglobin; Haptoglobin alpha chain; Haptoglobin beta chain |
| 275 | P00739 | HPTR | Haptoglobin-related protein |
| 276 | P00740 | FA9 | Coagulation factor IX; Coagulation factor IXa heavy chain |
| 277 | P00747 | PLMN | Activation peptide; Angiostatin; Plasmin heavy chain A |
| 278 | P00751 | CFAB | Complement factor B; Complement factor B Ba fragment |
| 279 | P00915 | CAH1 | Carbonic anhydrase 1 |
| 280 | P00918 | CAH2 | Carbonic anhydrase 2 |
| 281 | P00966 | ASSY | Argininosuccinate synthase |
| 282 | P00995 | ISK1 | Pancreatic secretory trypsin inhibitor |
| 283 | P01008 | ANT3 | Antithrombin-III |
| 284 | P01009 | A1AT | Alpha-1-antitrypsin; Short peptide from AAT |
| 285 | P01011 | AACT | Alpha-1-antichymotrypsin; Alpha-1-antichymotrypsin His-Pro-less |
| 286 | P01023 | A2MG | Alpha-2-macroglobulin |
| 287 | P01024 | CO3 | Acylation stimulating protein; C3a anaphylatoxin; C3-beta-c; Complement |
| | | | C3 |
| 288 | P01034 | CYTC | Cystatin-C |
| 289 | P01042 | KNG1 | Bradykinin; Kininogen-1; Kininogen-1 heavy chain; Kininogen-1 light chain |
| 290 | P01189 | COLI | Beta-endorphin; Corticotropin; Corticotropin-like intermediary peptide |
| 291 | P01275 | GLUC | Glicentin; Glicentin-related polypeptide; Glucagon; Glucagon-like peptide 1 |
| 292 | P01282 | VIP | Intestinal peptide PHM-27; Intestinal peptide PHV-42; Vasoactive |
| | | | intestinal peptide |
| 293 | P01298 | PAHO | Pancreatic hormone; Pancreatic icosapeptide; Pancreatic prohormone |
| 294 | P01308 | INS | Insulin; Insulin A chain; Insulin B chain |
| 295 | P01591 | IGJ | Immunoglobulin J chain |
| 296 | P01833 | PIGR | Polymeric immunoglobulin receptor; Secretory component |
| 297 | P01834 | IGKC | Ig kappa chain C region |
| 298 | P01857 | IGHG1 | Ig gamma-1 chain C region |
| 299 | P01859 | IGHG2 | Ig gamma-2 chain C region |
| 300 | P01860 | IGHG3 | Ig gamma-3 chain C region |
| 301 | P01861 | IGHG4 | Ig gamma-4 chain C region |
| 302 | P01876 | IGHA1 | Ig alpha-1 chain C region |
| 303 | P02042 | HBD | Hemoglobin subunit delta |
| 304 | P02452 | CO1A1 | Collagen alpha-1(I) chain |
| 305 | P02461 | CO3A1 | Collagen alpha-1(III) chain |
| 306 | P02462 | CO4A1 | Arresten; Collagen alpha-1(IV) chain |
| 307 | P02511 | CRYAB | Alpha-crystallin B chain |
| 308 | P02545 | LMNA | Lamin-A/C; Prelamin-A/C |
| 309 | P02647 | APOA1 | Apolipoprotein A-I; Proapolipoprotein A-I; Truncated apolipoprotein A-I |
| 310 | P02652 | APOA2 | Apolipoprotein A-II; Proapolipoprotein A-II; Truncated apolipoprotein A-II |
| 311 | P02671 | FIBA | Fibrinogen alpha chain; Fibrinopeptide A |
| 312 | P02675 | FIBB | Fibrinogen beta chain; Fibrinopeptide B |
| 313 | P02679 | FIBG | Fibrinogen gamma chain |
| 314 | P02686 | MBP | Myelin basic protein |
| 315 | P02689 | MYP2 | Myelin P2 protein |
| 316 | P02748 | CO9 | Complement component C9; Complement component C9a; Complement |
| | | | component C9b |
| 317 | P02749 | APOH | Beta-2-glycoprotein 1 |
| 318 | P02750 | A2GL | Leucine-rich alpha-2-glycoprotein |
| 319 | P02751 | FINC | Anastellin; Fibronectin; Ugl-Y1; Ugl-Y2; Ugl-Y3 |
| 320 | P02760 | AMBP | Alpha-1-microglobulin; Inter-alpha-trypsin inhibitor light chain; Protein |
| | | | AMBP; Trypstatin |
| 321 | P02763 | A1AG1 | Alpha-1-acid glycoprotein 1 |
| 322 | P02765 | FETUA | Alpha-2-HS-glycoprotein; Alpha-2-HS-glycoprotein chain A |
| 323 | P02766 | TTHY | Transthyretin |
| 324 | P02768 | ALBU | Serum albumin |
| 325 | P02774 | VTDB | Vitamin D-binding protein |
| 326 | P02787 | TRFE | Serotransferrin |
| 327 | P02788 | TRFL | Kaliocin-1; Lactoferricin-H; Lactoferroxin-A; Lactoferroxin-B; Lactoferroxin- |
| | | | C |
| 328 | P02790 | HEMO | Hemopexin |
| 329 | P02794 | FRIH | Ferritin heavy chain; Ferritin heavy chain, N-terminally processed |
| 330 | P02795 | MT2 | Metallothionein-2 |
| 331 | P03950 | ANGI | Angiogenin |
| 332 | P04004 | VTNC | Somatomedin-B; Vitronectin; Vitronectin V10 subunit; Vitronectin V65 |
| | | | subunit |
| 333 | P04040 | CATA | Catalase |
| 334 | P04054 | PA21B | Phospholipase A2 |
| 335 | P04075 | ALDOA | Fructose-bisphosphate aldolase A |
| 336 | P04080 | CYTB | Cystatin-B |
| 337 | P04083 | ANXA1 | Annexin A1 |
| 338 | P04118 | COL | Colipase |
| 339 | P04155 | TFF1 | Trefoil factor 1 |
| 340 | P04179 | SODM | Superoxide dismutase [Mn], mitochondrial |
| 341 | P04196 | HRG | Histidine-rich glycoprotein |
| 342 | P04217 | A1BG | Alpha-1B-glycoprotein |
| 343 | P04264 | K2C1 | Keratin, type II cytoskeletal 1 |
| 344 | P04406 | G3P | Glyceraldehyde-3-phosphate dehydrogenase |
| 345 | P04632 | CPNS1 | Calpain small subunit 1 |
| 346 | P04731 | MT1A | Metallothionein-1A |
| 347 | P04732 | MT1E | Metallothionein-1E |
| 348 | P04733 | MT1F | Metallothionein-1F |
| 349 | P04746 | AMYP | Pancreatic alpha-amylase |
| 350 | P04792 | HSPB1 | Heat shock protein beta-1 |
| 351 | P04843 | RPN1 | Dolichyl-diphosphooligosaccharide--protein glycosyltransferase subunit 1 |
| 352 | P04844 | RPN2 | Dolichyl-diphosphooligosaccharide--protein glycosyltransferase subunit 2 |
| 353 | P05023 | AT1A1 | Sodium/potassium-transporting ATPase subunit alpha-1 |
| 354 | P05026 | AT1B1 | Sodium/potassium-transporting ATPase subunit beta-1 |
| 355 | P05060 | SCG1 | CCB peptide; GAWK peptide; PE-11; Secretogranin-1 |
| 356 | P05091 | ALDH2 | Aldehyde dehydrogenase, mitochondrial |
| 357 | P05109 | S10A8 | Protein S100-A8; Protein S100-A8, N-terminally processed |
| 358 | P05114 | HMGN1 | Non-histone chromosomal protein HMG-14 |
| 359 | P05141 | ADT2 | ADP/ATP translocase 2; ADP/ATP translocase 2, N-terminally processed |
| 360 | P05162 | LEG2 | Galectin-2 |
| 361 | P05164 | PERM | 84 kDa myeloperoxidase; 89 kDa myeloperoxidase; Myeloperoxidase |
| 362 | P05165 | PCCA | Propionyl-CoA carboxylase alpha chain, mitochondrial |
| 363 | P05166 | PCCB | Propionyl-CoA carboxylase beta chain, mitochondrial |
| 364 | P05186 | PPBT | Alkaline phosphatase, tissue-nonspecific isozyme |
| 365 | P05198 | IF2A | Eukaryotic translation initiation factor 2 subunit 1 |
| 366 | P05204 | HMGN2 | Non-histone chromosomal protein HMG-17 |
| 367 | P05362 | ICAM1 | Intercellular adhesion molecule 1 |
| 368 | P05386 | RLA1 | 60S acidic ribosomal protein P1 |
| 369 | P05387 | RLA2 | 60S acidic ribosomal protein P2 |
| 370 | P05388 | RLA0 | 60S acidic ribosomal protein P0 |
| 371 | P05408 | 7B2 | C-terminal peptide; Neuroendocrine protein 7B2; N-terminal peptide |
| 372 | P05413 | FABPH | Fatty acid-binding protein, heart |
| 373 | P05451 | REG1A | Lithostathine-1-alpha |
| 374 | P05455 | LA | Lupus La protein |
| 375 | P05556 | ITB1 | Integrin beta-1 |
| 376 | P05783 | K1C18 | Keratin, type I cytoskeletal 18 |
| 377 | P05787 | K2C8 | Keratin, type II cytoskeletal 8 |
| 378 | P05997 | CO5A2 | Collagen alpha-2(V) chain |
| 379 | P06213 | INSR | Insulin receptor; Insulin receptor subunit alpha; Insulin receptor subunit |
| | | | beta |
| 380 | P06396 | GELS | Gelsolin |
| 381 | P06454 | PTMA | Prothymosin alpha; Prothymosin alpha, N-terminally processed; Thymosin |
| | | | alpha-1 |
| 382 | P06576 | ATPB | ATP synthase subunit beta, mitochondrial |
| 383 | P06702 | S10A9 | Protein S100-A9 |
| 384 | P06703 | S10A6 | Protein S100-A6 |
| 385 | P06730 | IF4E | Eukaryotic translation initiation factor 4E |
| 386 | P06733 | ENOA | Alpha-enolase |
| 387 | P06744 | G6PI | Glucose-6-phosphate isomerase |
| 388 | P06748 | NPM | Nucleophosmin |
| 389 | P06753 | TPM3 | Tropomyosin alpha-3 chain |
| 390 | P06870 | KLK1 | Kallikrein-1 |
| 391 | P07099 | HYEP | Epoxide hydrolase 1 |
| 392 | P07108 | ACBP | Acyl-CoA-binding protein |
| 393 | P07148 | FABPL | Fatty acid-binding protein, liver |
| 394 | P07195 | LDHB | L-lactate dehydrogenase B chain |
| 395 | P07203 | GPX1 | Glutathione peroxidase 1 |
| 396 | P07237 | PDIA1 | Protein disulfide-isomerase |
| 397 | P07305 | H10 | Histone H1.0; Histone H1.0, N-terminally processed |
| 398 | P07311 | ACYP1 | Acylphosphatase-1 |
| 399 | P07339 | CATD | Cathepsin D; Cathepsin D heavy chain; Cathepsin D light chain |
| 400 | P07355 | ANXA2 | Annexin A2 |
| 401 | P07437 | TBB5 | Tubulin beta chain |
| 402 | P07477 | TRY1 | Alpha-trypsin chain 1 ; Alpha-trypsin chain 2; Trypsin-1 |
| 403 | P07478 | TRY2 | Trypsin-2 |
| 404 | P07585 | PGS2 | Decorin |
| 405 | P07602 | SAP | Prosaposin; Saposin-A; Saposin-B; Saposin-B-Val; Saposin-C; Saposin-D |
| 406 | P07711 | CATL1 | Cathepsin L1; Cathepsin L1 heavy chain; Cathepsin L1 light chain |
| 407 | P07737 | PROF1 | Profilin-1 |
| 408 | P07741 | APT | Adenine phosphoribosyltransferase |
| 409 | P07814 | SYEP | Bifunctional glutamate/proline-tRNA ligase; Glutamate-tRNA ligase |
| 410 | P07858 | CATB | Cathepsin B; Cathepsin B heavy chain; Cathepsin B light chain |
| 411 | P07900 | HS90A | Heat shock protein HSP 90-alpha |
| 412 | P07910 | HNRPC | Heterogeneous nuclear ribonucleoproteins C1/C2 |
| 413 | P07919 | QCR6 | Cytochrome b-c1 complex subunit 6, mitochondrial |
| 414 | P07942 | LAMB1 | Laminin subunit beta-1 |
| 415 | P07951 | TPM2 | Tropomyosin beta chain |
| 416 | P07954 | FUMH | Fumarate hydratase, mitochondrial |
| 417 | P07996 | TSP1 | Thrombospondin-1 |
| 418 | P07998 | RNAS1 | Ribonuclease pancreatic |
| 419 | P08047 | SP1 | Transcription factor Sp1 |
| 420 | P08123 | CO1A2 | Collagen alpha-2(I) chain |
| 421 | P08133 | ANXA6 | Annexin A6 |
| 422 | P08217 | CEL2A | Chymotrypsin-like elastase family member 2A |
| 423 | P08218 | CEL2B | Chymotrypsin-like elastase family member 2B |
| 424 | P08238 | HS90B | Heat shock protein HSP 90-beta |
| 425 | P08240 | SRPRA | Signal recognition particle receptor subunit alpha |
| 426 | P08246 | ELNE | Neutrophil elastase |
| 427 | P08263 | GSTA1 | Glutathione S-transferase A1; Glutathione S-transferase A1, N-terminally |
| | | | processed |
| 428 | P08294 | SODE | Extracellular superoxide dismutase [Cu—Zn] |
| 429 | P08311 | CATG | Cathepsin G |
| 430 | P08559 | ODPA | Pyruvate dehydrogenase E1 component subunit alpha, somatic form, |
| | | | mitochondrial |
| 431 | P08572 | CO4A2 | Canstatin; Collagen alpha-2(IV) chain |
| 432 | P08621 | RU17 | U1 small nuclear ribonucleoprotein 70 kDa |
| 433 | P08670 | VIME | Vimentin |
| 434 | P08708 | RS17 | 40S ribosomal protein S17 |
| 435 | P08727 | K1C19 | Keratin, type I cytoskeletal 19 |
| 436 | P08729 | K2C7 | Keratin, type II cytoskeletal 7 |
| 437 | P08758 | ANXA5 | Annexin A5 |
| 438 | P08861 | CEL3B | Chymotrypsin-like elastase family member 3B |
| 439 | P08865 | RSSA | 40S ribosomal protein SA |
| 440 | P09012 | SNRPA | U1 small nuclear ribonucleoprotein A |
| 441 | P09093 | CEL3A | Chymotrypsin-like elastase family member 3A |
| 442 | P09110 | THIK | 3-ketoacyl-CoA thiolase, peroxisomal |
| 443 | P09132 | SRP19 | Signal recognition particle 19 kDa protein |
| 444 | P09210 | GSTA2 | Glutathione S-transferase A2 |
| 445 | P09211 | GSTP1 | Glutathione S-transferase P |
| 446 | P09234 | RU1C | U1 small nuclear ribonucleoprotein C |
| 447 | P09327 | VILI | Villin-1 |
| 448 | P09382 | LEG1 | Galectin-1 |
| 449 | P09429 | HMGB1 | High mobility group protein B1 |
| 450 | P09455 | RET1 | Retinol-binding protein 1 |
| 451 | P09467 | F16P1 | Fructose-1,6-bisphosphatase 1 |
| 452 | P09493 | TPM1 | Tropomyosin alpha-1 chain |
| 453 | P09496 | CLCA | Clathrin light chain A |
| 454 | P09497 | CLCB | Clathrin light chain B |
| 455 | P09525 | ANXA4 | Annexin A4 |
| 456 | P09622 | DLDH | Dihydrolipoyl dehydrogenase, mitochondrial |
| 457 | P09651 | ROA1 | Heterogeneous nuclear ribonucleoprotein A1 |
| 458 | P09661 | RU2A | U2 small nuclear ribonucleoprotein A′ |
| 459 | P09669 | COX6C | Cytochrome c oxidase subunit 6C |
| 460 | P09758 | TACD2 | Tumor-associated calcium signal transducer 2 |
| 461 | P09874 | PARP1 | Poly [ADP-ribose] polymerase 1 |
| 462 | P09936 | UCHL1 | Ubiquitin carboxyl-terminal hydrolase isozyme L1 |
| 463 | P09960 | LKHA4 | Leukotriene A-4 hydrolase |
| 464 | P09972 | ALDOC | Fructose-bisphosphate aldolase C |
| 465 | P0C0L4 | CO4A | C4a anaphylatoxin; C4b-A; C4d-A |
| 466 | P0C0S5 | H2AZ | Histone H2A.Z |
| 467 | P0CG12 | CTF8A | Chromosome transmission fidelity protein 8 homolog isoform 2 |
| 468 | P0CG39 | POTEJ | POTE ankyrin domain family member J |
| 469 | P0DMV8 | HS71A | Heat shock 70 kDa protein 1A |
| 470 | P0DN79 | CBSL | |
| 471 | P10109 | ADX | Adrenodoxin, mitochondrial |
| 472 | P10253 | LYAG | 70 kDa lysosomal alpha-glucosidase |
| 473 | P10412 | H14 | Histone H1.4 |
| 474 | P10451 | OSTP | Osteopontin |
| 475 | P10515 | ODP2 | Dihydrolipoyllysine-residue acetyltransferase component of pyruvate |
| | | | dehydrogenase complex, mitochondrial |
| 476 | P10599 | THIO | Thioredoxin |
| 477 | P10606 | COX5B | Cytochrome c oxidase subunit 5B, mitochondrial |
| 478 | P10620 | MGST1 | Microsomal glutathione S-transferase 1 |
| 479 | P10636 | TAU | Microtubule-associated protein tau |
| 480 | P10643 | CO7 | Complement component C7 |
| 481 | P10645 | CMGA | AL-11; Catestatin; Chromogranin-A; EA-92; ER-37; ES-43; GE-25; GR- |
| | | | 44; GV-19; LF-19; Pancreastatin |
| 482 | P10768 | ESTD | S-formylglutathione hydrolase |
| 483 | P10809 | CH60 | 60 kDa heat shock protein, mitochondrial |
| 484 | P10909 | CLUS | Clusterin; Clusterin alpha chain; Clusterin beta chain |
| 485 | P10997 | IAPP | Islet amyloid polypeptide |
| 486 | P11021 | GRP78 | 78 kDa glucose-regulated protein |
| 487 | P11047 | LAMC1 | Laminin subunit gamma-1 |
| 488 | P11117 | PPAL | Lysosomal acid phosphatase |
| 489 | P11137 | MTAP2 | Microtubule-associated protein 2 |
| 490 | P11142 | HSP7C | Heat shock cognate 71 kDa protein |
| 491 | P11171 | 41 | Protein 4.1 |
| 492 | P11177 | ODPB | Pyruvate dehydrogenase E1 component subunit beta, mitochondrial |
| 493 | P11182 | ODB2 | Lipoamide acyltransferase component of branched-chain alpha-keto acid |
| | | | dehydrogenase complex, mitochondrial |
| 494 | P11279 | LAMP1 | Lysosome-associated membrane glycoprotein 1 |
| 495 | P11310 | ACADM | Medium-chain specific acyl-CoA dehydrogenase, mitochondrial |
| 496 | P11387 | TOP1 | DNA topoisomerase 1 |
| 497 | P11532 | DMD | Dystrophin |
| 498 | P11586 | C1TC | C-1-tetrahydrofolate synthase, cytoplasmic |
| 499 | P11766 | ADHX | Alcohol dehydrogenase class-3 |
| 500 | P11940 | PABP1 | Polyadenylate-binding protein 1 |
| 501 | P12004 | PCNA | Proliferating cell nuclear antigen |
| 502 | P12109 | CO6A1 | Collagen alpha-1 (VI) chain |
| 503 | P12110 | CO6A2 | Collagen alpha-2(VI) chain |
| 504 | P12111 | CO6A3 | Collagen alpha-3(VI) chain |
| 505 | P12235 | ADT1 | ADP/ATP translocase 1 |
| 506 | P12236 | ADT3 | ADP/ATP translocase 3; ADP/ATP translocase 3, N-terminally processed |
| 507 | P12268 | IMDH2 | Inosine-5′-monophosphate dehydrogenase 2 |
| 508 | P12270 | TPR | Nucleoprotein TPR |
| 509 | P12277 | KCRB | Creatine kinase B-type |
| 510 | P12429 | ANXA3 | Annexin A3 |
| 511 | P12694 | ODBA | 2-oxoisovalerate dehydrogenase subunit alpha, mitochondrial |
| 512 | P12814 | ACTN1 | Alpha-actinin-1 |
| 513 | P12830 | CADH1 | Cadherin-1; E-Cad/CTF1; E-Cad/CTF2; E-Cad/CTF3 |
| 514 | P12956 | XRCC6 | X-ray repair cross-complementing protein 6 |
| 515 | P13010 | XRCC5 | X-ray repair cross-complementing protein 5 |
| 516 | P13073 | COX41 | Cytochrome c oxidase subunit 4 isoform 1, mitochondrial |
| 517 | P13473 | LAMP2 | Lysosome-associated membrane glycoprotein 2 |
| 518 | P13489 | RINI | Ribonuclease inhibitor |
| 519 | P13521 | SCG2 | Manserin; Secretogranin-2; Secretoneurin |
| 520 | P13639 | EF2 | Elongation factor 2 |
| 521 | P13640 | MT1G | Metallothionein-1 G |
| 522 | P13645 | K1C10 | Keratin, type I cytoskeletal 10 |
| 523 | P13647 | K2C5 | Keratin, type II cytoskeletal 5 |
| 524 | P13667 | PDIA4 | Protein disulfide-isomerase A4 |
| 525 | P13693 | TCTP | Translationally-controlled tumor protein |
| 526 | P13716 | HEM2 | Delta-aminolevulinic acid dehydratase |
| 527 | P13796 | PLSL | Plastin-2 |
| 528 | P13797 | PLST | Plastin-3 |
| 529 | P13804 | ETFA | Electron transfer flavoprotein subunit alpha, mitochondrial |
| 530 | P13861 | KAP2 | cAMP-dependent protein kinase type II-alpha regulatory subunit |
| 531 | P13984 | T2FB | General transcription factor IIF subunit 2 |
| 532 | P13987 | CD59 | CD59 glycoprotein |
| 533 | P14174 | MIF | Macrophage migration inhibitory factor |
| 534 | P14209 | CD99 | CD99 antigen |
| 535 | P14314 | GLU2B | Glucosidase 2 subunit beta |
| 536 | P14317 | HCLS1 | Hematopoietic lineage cell-specific protein |
| 537 | P14406 | CX7A2 | Cytochrome c oxidase subunit 7A2, mitochondrial |
| 538 | P14543 | NID1 | Nidogen-1 |
| 539 | P14550 | AK1A1 | Alcohol dehydrogenase [NADP(+)] |
| 540 | P14555 | PA2GA | Phospholipase A2, membrane associated |
| 541 | P14618 | KPYM | Pyruvate kinase PKM |
| 542 | P14621 | ACYP2 | Acylphosphatase-2 |
| 543 | P14625 | ENPL | Endoplasmin |
| 544 | P14854 | CX6B1 | Cytochrome c oxidase subunit 6B1 |
| 545 | P14866 | HNRPL | Heterogeneous nuclear ribonucleoprotein L |
| 546 | P14868 | SYDC | Aspartate-tRNA ligase, cytoplasmic |
| 547 | P14923 | PLAK | Junction plakoglobin |
| 548 | P14927 | QCR7 | Cytochrome b-c1 complex subunit 7 |
| 549 | P15085 | CBPA1 | Carboxypeptidase A1 |
| 550 | P15086 | CBPB1 | Carboxypeptidase B |
| 551 | P15088 | CBPA3 | Mast cell carboxypeptidase A |
| 552 | P15090 | FABP4 | Fatty acid-binding protein, adipocyte |
| 553 | P15104 | GLNA | Glutamine synthetase |
| 554 | P15144 | AMPN | Aminopeptidase N |
| 555 | P15170 | ERF3A | Eukaryotic peptide chain release factor GTP-binding subunit ERF3A |
| 556 | P15311 | EZRI | Ezrin |
| 557 | P15374 | UCHL3 | Ubiquitin carboxyl-terminal hydrolase isozyme L3 |
| 558 | P15531 | NDKA | Nucleoside diphosphate kinase A |
| 559 | P15880 | RS2 | 40S ribosomal protein S2 |
| 560 | P15924 | DESP | Desmoplakin |
| 561 | P15927 | RFA2 | Replication protein A 32 kDa subunit |
| 562 | P16070 | CD44 | CD44 antigen |
| 563 | P16144 | ITB4 | Integrin beta-4 |
| 564 | P16152 | CBR1 | Carbonyl reductase [NADPH] 1 |
| 565 | P16219 | ACADS | Short-chain specific acyl-CoA dehydrogenase, mitochondrial |
| 566 | P16220 | CREB1 | Cyclic AMP-responsive element-binding protein 1 |
| 567 | P16233 | LIPP | Pancreatic triacylglycerol lipase |
| 568 | P16333 | NCK1 | Cytoplasmic protein NCK1 |
| 569 | P16401 | H15 | Histone H1.5 |
| 570 | P16402 | H13 | Histone H1.3 |
| 571 | P16403 | H12 | Histone H1.2 |
| 572 | P16422 | EPCAM | Epithelial cell adhesion molecule |
| 573 | P16444 | DPEP1 | Dipeptidase 1 |
| 574 | P16455 | MGMT | Methylated-DNA-protein-cysteine methyltransferase |
| 575 | P16615 | AT2A2 | Sarcoplasmic/endoplasmic reticulum calcium ATPase 2 |
| 576 | P16870 | CBPE | Carboxypeptidase E |
| 577 | P16949 | STMN1 | Stathmin |
| 578 | P16989 | YBOX3 | Y-box-binding protein 3 |
| 579 | P17026 | ZNF22 | Zinc finger protein 22 |
| 580 | P17096 | HMGA1 | High mobility group protein HMG-I/HMG-Y |
| 581 | P17480 | UBF1 | Nucleolar transcription factor 1 |
| 582 | P17538 | CTRB1 | Chymotrypsin B chain A; Chymotrypsin B chain B; Chymotrypsin B chain |
| | | | C; Chymotrypsinogen B |
| 583 | P17544 | ATF7 | Cyclic AMP-dependent transcription factor ATF-7 |
| 584 | P17655 | CAN2 | Calpain-2 catalytic subunit |
| 585 | P17661 | DESM | Desmin |
| 586 | P17677 | NEUM | Neuromodulin |
| 587 | P17844 | DDX5 | Probable ATP-dependent RNA helicase DDX5 |
| 588 | P17931 | LEG3 | Galectin-3 |
| 589 | P17980 | PRS6A | 26S protease regulatory subunit 6A |
| 590 | P17987 | TCPA | T-complex protein 1 subunit alpha |
| 591 | P18065 | IBP2 | Insulin-like growth factor-binding protein 2 |
| 592 | P18077 | RL35A | 60S ribosomal protein L35a |
| 593 | P18085 | ARF4 | ADP-ribosylation factor 4 |
| 594 | P18124 | RL7 | 60S ribosomal protein L7 |
| 595 | P18206 | VINC | Vinculin |
| 596 | P18583 | SON | Protein SON |
| 597 | P18615 | NELFE | Negative elongation factor E |
| 598 | P18621 | RL17 | 60S ribosomal protein L17 |
| 599 | P18669 | PGAM1 | Phosphoglycerate mutase 1 |
| 600 | P18754 | RCC1 | Regulator of chromosome condensation |
| 601 | P18859 | ATP5J | ATP synthase-coupling factor 6, mitochondrial |
| 602 | P18887 | XRCC1 | DNA repair protein XRCC1 |
| 603 | P19338 | NUCL | Nucleolin |
| 604 | P19404 | NDUV2 | NADH dehydrogenase [ubiquinone] flavoprotein 2, mitochondrial |
| 605 | P19623 | SPEE | Spermidine synthase |
| 606 | P19652 | A1AG2 | Alpha-1-acid glycoprotein 2 |
| 607 | P19823 | ITIH2 | Inter-alpha-trypsin inhibitor heavy chain H2 |
| 608 | P19835 | CEL | Bile salt-activated lipase |
| 609 | P19961 | AMY2B | Alpha-amylase 2B |
| 610 | P20042 | IF2B | Eukaryotic translation initiation factor 2 subunit 2 |
| 611 | P20160 | CAP7 | Azurocidin |
| 612 | P20231 | TRYB2 | Tryptase beta-2 |
| 613 | P20290 | BTF3 | Transcription factor BTF3 |
| 614 | P20618 | PSB1 | Proteasome subunit beta type-1 |
| 615 | P20674 | COX5A | Cytochrome c oxidase subunit 5A, mitochondrial |
| 616 | P20700 | LMNB1 | Lamin-B1 |
| 617 | P20774 | MIME | Mimecan |
| 618 | P20810 | ICAL | Calpastatin |
| 619 | P20933 | ASPG | Glycosylasparaginase alpha chain; Glycosylasparaginase beta chain |
| 620 | P20962 | PTMS | Parathymosin |
| 621 | P21266 | GSTM3 | Glutathione S-transferase Mu 3 |
| 622 | P21281 | VATB2 | V-type proton ATPase subunit B, brain isoform |
| 623 | P21291 | CSRP1 | Cysteine and glycine-rich protein 1 |
| 624 | P21333 | FLNA | Filamin-A |
| 625 | P21399 | ACOC | Cytoplasmic aconitate hydratase |
| 626 | P21580 | TNAP3 | A20p37; A20p50; Tumor necrosis factor alpha-induced protein 3 |
| 627 | P21695 | GPDA | Glycerol-3-phosphate dehydrogenase [NAD(+)], cytoplasmic |
| 628 | P21796 | VDAC1 | Voltage-dependent anion-selective channel protein 1 |
| 629 | P21810 | PGS1 | Biglycan |
| 630 | P21912 | SDHB | Succinate dehydrogenase [ubiquinone] iron-sulfur subunit, mitochondrial |
| 631 | P21953 | ODBB | 2-oxoisovalerate dehydrogenase subunit beta, mitochondrial |
| 632 | P21964 | COMT | Catechol O-methyltransferase |
| 633 | P21980 | TGM2 | Protein-glutamine gamma-glutamyltransferase 2 |
| 634 | P22033 | MUTA | Methylmalonyl-CoA mutase, mitochondrial |
| 635 | P22059 | OSBP1 | Oxysterol-binding protein 1 |
| 636 | P22061 | PIMT | Protein-L-isoaspartate(D-aspartate) O-methyltransferase |
| 637 | P22087 | FBRL | rRNA 2′-O-methyltransferase fibrillarin |
| 638 | P22102 | PUR2 | Phosphoribosylamine--glycine ligase; Phosphoribosylformylglycinamidine |
| | | | cyclo-ligase |
| 639 | P22105 | TENX | Tenascin-X |
| 640 | P22307 | NLTP | Non-specific lipid-transfer protein |
| 641 | P22314 | UBA1 | Ubiquitin-like modifier-activating enzyme 1 |
| 642 | P22392 | NDKB | Nucleoside diphosphate kinase B |
| 643 | P22413 | ENPP1 | Alkaline phosphodiesterase I; Ectonucleotide |
| | | | pyrophosphatase/phosphodiesterase family member 1 |
| 644 | P22570 | ADRO | NADPH:adrenodoxin oxidoreductase, mitochondrial |
| 645 | P22626 | ROA2 | Heterogeneous nuclear ribonucleoproteins A2/B1 |
| 646 | P22695 | QCR2 | Cytochrome b-c1 complex subunit 2, mitochondrial |
| 647 | P22748 | CAH4 | Carbonic anhydrase 4 |
| 648 | P22894 | MMP8 | Neutrophil collagenase |
| 649 | P23142 | FBLN1 | Fibulin-1 |
| 650 | P23193 | TCEA1 | Transcription elongation factor A protein 1 |
| 651 | P23229 | ITA6 | Integrin alpha-6; Integrin alpha-6 heavy chain; Integrin alpha-6 light |
| | | | chain; Processed integrin alpha-6 |
| 652 | P23246 | SFPQ | Splicing factor, proline- and glutamine-rich |
| 653 | P23284 | PPIB | Peptidyl-prolyl cis-trans isomerase B |
| 654 | P23297 | S10A1 | Protein S100-A1 |
| 655 | P23381 | SYWC | T1-TrpRS; T2-TrpRS; Tryptophan-tRNA ligase, cytoplasmic |
| 656 | P23396 | RS3 | 40S ribosomal protein S3 |
| 657 | P23434 | GCSH | Glycine cleavage system H protein, mitochondrial |
| 658 | P23497 | SP100 | Nuclear autoantigen Sp-100 |
| 659 | P23526 | SAHH | Adenosylhomocysteinase |
| 660 | P23528 | COF1 | Cofilin-1 |
| 661 | P23588 | IF4B | Eukaryotic translation initiation factor 4B |
| 662 | P23946 | CMA1 | Chymase |
| 663 | P24043 | LAMA2 | Laminin subunit alpha-2 |
| 664 | P24534 | EF1B | Elongation factor 1-beta |
| 665 | P24539 | AT5F1 | ATP synthase F(0) complex subunit B1, mitochondrial |
| 666 | P24593 | IBP5 | Insulin-like growth factor-binding protein 5 |
| 667 | P24666 | PPAC | Low molecular weight phosphotyrosine protein phosphatase |
| 668 | P24752 | THIL | Acetyl-CoA acetyltransferase, mitochondrial |
| 669 | P24821 | TENA | Tenascin |
| 670 | P24844 | MYL9 | Myosin regulatory light polypeptide 9 |
| 671 | P24928 | RPB1 | DNA-directed RNA polymerase II subunit RPB1 |
| 672 | P25311 | ZA2G | Zinc-alpha-2-glycoprotein |
| 673 | P25325 | THTM | 3-mercaptopyruvate sulfurtransferase |
| 674 | P25398 | RS12 | 40S ribosomal protein S12 |
| 675 | P25490 | TYY1 | Transcriptional repressor protein YY1 |
| 676 | P25685 | DNJB1 | DnaJ homolog subfamily B member 1 |
| 677 | P25686 | DNJB2 | DnaJ homolog subfamily B member 2 |
| 678 | P25705 | ATPA | ATP synthase subunit alpha, mitochondrial |
| 679 | P25786 | PSA1 | Proteasome subunit alpha type-1 |
| 680 | P25787 | PSA2 | Proteasome subunit alpha type-2 |
| 681 | P25788 | PSA3 | Proteasome subunit alpha type-3 |
| 682 | P25789 | PSA4 | Proteasome subunit alpha type-4 |
| 683 | P25815 | S100P | Protein S100-P |
| 684 | P25940 | CO5A3 | Collagen alpha-3(V) chain |
| 685 | P26038 | MOES | Moesin |
| 686 | P26196 | DDX6 | Probable ATP-dependent RNA helicase DDX6 |
| 687 | P26232 | CTNA2 | Catenin alpha-2 |
| 688 | P26368 | U2AF2 | Splicing factor U2AF 65 kDa subunit |
| 689 | P26373 | RL13 | 60S ribosomal protein L13 |
| 690 | P26447 | S10A4 | Protein S100-A4 |
| 691 | P26583 | HMGB2 | High mobility group protein B2 |
| 692 | P26599 | PTBP1 | Polypyrimidine tract-binding protein 1 |
| 693 | P26639 | SYTC | Threonine-tRNA ligase, cytoplasmic |
| 694 | P26640 | SYVC | Valine-tRNA ligase |
| 695 | P26641 | EF1G | Elongation factor 1-gamma |
| 696 | P26885 | FKBP2 | Peptidyl-prolyl cis-trans isomerase FKBP2 |
| 697 | P27144 | KAD4 | Adenylate kinase 4, mitochondrial |
| 698 | P27348 | 1433T | 14-3-3 protein theta |
| 699 | P27635 | RL10 | 60S ribosomal protein L10 |
| 700 | P27695 | APEX1 | DNA-(apurinic or apyrimidinic site) lyase; DNA-(apurinic or apyrimidinic |
| | | | site) lyase, mitochondrial |
| 701 | P27708 | PYR1 | Aspartate carbamoyltransferase; CAD protein; Dihydroorotase |
| 702 | P27797 | CALR | Calreticulin |
| 703 | P27816 | MAP4 | Microtubule-associated protein 4 |
| 704 | P27824 | CALX | Calnexin |
| 705 | P28066 | PSA5 | Proteasome subunit alpha type-5 |
| 706 | P28070 | PSB4 | Proteasome subunit beta type-4 |
| 707 | P28072 | PSB6 | Proteasome subunit beta type-6 |
| 708 | P28074 | PSB5 | Proteasome subunit beta type-5 |
| 709 | P28289 | TMOD1 | Tropomodulin-1 |
| 710 | P28290 | SSFA2 | Sperm-specific antigen 2 |
| 711 | P28331 | NDUS1 | NADH-ubiquinone oxidoreductase 75 kDa subunit, mitochondrial |
| 712 | P28799 | GRN | Acrogranin; Granulin-1; Granulin-2; Granulin-3; Granulin-4; Granulin- |
| | | | 5; Granulin-6; Granulin-7; Granulins; Paragranulin |
| 713 | P28838 | AMPL | Cytosol aminopeptidase |
| 714 | P28906 | CD34 | Hematopoietic progenitor cell antigen CD34 |
| 715 | P29084 | T2EB | Transcription initiation factor HE subunit beta |
| 716 | P29144 | TPP2 | Tripeptidyl-peptidase 2 |
| 717 | P29279 | CTGF | Connective tissue growth factor |
| 718 | P29375 | KDM5A | Lysine-specific demethylase 5A |
| 719 | P29400 | CO4A5 | Collagen alpha-5(IV) chain |
| 720 | P29401 | TKT | Transketolase |
| 721 | P29536 | LMOD1 | Leiomodin-1 |
| 722 | P29558 | RBMS1 | RNA-binding motif, single-stranded-interacting protein 1 |
| 723 | P29590 | PML | Protein PML |
| 724 | P29692 | EF1D | Elongation factor 1-delta |
| 725 | P29966 | MARCS | Myristoylated alanine-rich C-kinase substrate |
| 726 | P30038 | AL4A1 | Delta-1-pyrroline-5-carboxylate dehydrogenase, mitochondrial |
| 727 | P30039 | PBLD | Phenazine biosynthesis-like domain-containing protein |
| 728 | P30040 | ERP29 | Endoplasmic reticulum resident protein 29 |
| 729 | P30041 | PRDX6 | Peroxiredoxin-6 |
| 730 | P30042 | ES1 | ES1 protein homolog, mitochondrial |
| 731 | P30043 | BLVRB | Flavin reductase (NADPH) |
| 732 | P30044 | PRDX5 | Peroxiredoxin-5, mitochondrial |
| 733 | P30046 | DOPD | D-dopachrome decarboxylase |
| 734 | P30048 | PRDX3 | Thioredoxin-dependent peroxide reductase, mitochondrial |
| 735 | P30049 | ATPD | ATP synthase subunit delta, mitochondrial |
| 736 | P30050 | RL12 | 60S ribosomal protein L12 |
| 737 | P30084 | ECHM | Enoyl-CoA hydratase, mitochondrial |
| 738 | P30085 | KCY | UMP-CMP kinase |
| 739 | P30086 | PEBP1 | Hippocampal cholinergic neurostimulating |
| | | | peptide; Phosphatidylethanolamine-binding protein 1 |
| 740 | P30101 | PDIA3 | Protein disulfide-isomerase A3 |
| 741 | P30405 | PPIF | Peptidyl-prolyl cis-trans isomerase F, mitochondrial |
| 742 | P30419 | NMT1 | Glycylpeptide N-tetradecanoyltransferase 1 |
| 743 | P30519 | HMOX2 | Heme oxygenase 2 |
| 744 | P30533 | AMRP | Alpha-2-macroglobulin receptor-associated protein |
| 745 | P30622 | CLIP1 | CAP-Gly domain-containing linker protein 1 |
| 746 | P30626 | SORCN | Sorcin |
| 747 | P30740 | ILEU | Leukocyte elastase inhibitor |
| 748 | P30837 | AL1B1 | Aldehyde dehydrogenase X, mitochondrial |
| 749 | P31040 | SDHA | Succinate dehydrogenase [ubiquinone] flavoprotein subunit, |
| | | | mitochondrial |
| 750 | P31146 | COR1A | Coronin-1A |
| 751 | P31150 | GDIA | Rab GDP dissociation inhibitor alpha |
| 752 | P31153 | METK2 | S-adenosylmethionine synthase isoform type-2 |
| 753 | P31689 | DNJA1 | DnaJ homolog subfamily A member 1 |
| 754 | P31930 | QCR1 | Cytochrome b-c1 complex subunit 1, mitochondrial |
| 755 | P31937 | 3HIDH | 3-hydroxyisobutyrate dehydrogenase, mitochondrial |
| 756 | P31939 | PUR9 | Bifunctional purine biosynthesis protein PURH; IMP cyclohydrolase |
| 757 | P31942 | HNRH3 | Heterogeneous nuclear ribonucleoprotein H3 |
| 758 | P31943 | HNRH1 | Heterogeneous nuclear ribonucleoprotein H; Heterogeneous nuclear |
| | | | ribonucleoprotein H, N-terminally processed |
| 759 | P31946 | 1433B | 14-3-3 protein beta/alpha; 14-3-3 protein beta/alpha, N-terminally |
| | | | processed |
| 760 | P31948 | STIP1 | Stress-induced-phosphoprotein 1 |
| 761 | P31949 | S10AB | Protein S100-A11; Protein S100-A11, N-terminally processed |
| 762 | P32119 | PRDX2 | Peroxiredoxin-2 |
| 763 | P32245 | MC4R | Melanocortin receptor 4 |
| 764 | P32322 | P5CR1 | Pyrroline-5-carboxylate reductase 1, mitochondrial |
| 765 | P32519 | ELF1 | ETS-related transcription factor Elf-1 |
| 766 | P32929 | CGL | Cystathionine gamma-lyase |
| 767 | P32969 | RL9 | 60S ribosomal protein L9 |
| 768 | P33176 | KINH | Kinesin-1 heavy chain |
| 769 | P33240 | CSTF2 | Cleavage stimulation factor subunit 2 |
| 770 | P33241 | LSP1 | Lymphocyte-specific protein 1 |
| 771 | P33316 | DUT | Deoxyuridine 5′-triphosphate nucleotidohydrolase, mitochondrial |
| 772 | P34059 | GALNS | N-acetylgalactosamine-6-sulfatase |
| 773 | P34096 | RNAS4 | Ribonuclease 4 |
| 774 | P34897 | GLYM | Serine hydroxymethyltransferase, mitochondrial |
| 775 | P34913 | HYES | Bifunctional epoxide hydrolase 2; Cytosolic epoxide hydrolase 2; Lipid- |
| | | | phosphate phosphatase |
| 776 | P34932 | HSP74 | Heat shock 70 kDa protein 4 |
| 777 | P35030 | TRY3 | Trypsin-3 |
| 778 | P35080 | PROF2 | Profilin-2 |
| 779 | P35221 | CTNA1 | Catenin alpha-1 |
| 780 | P35222 | CTNB1 | Catenin beta-1 |
| 781 | P35232 | PHB | Prohibitin |
| 782 | P35237 | SPB6 | Serpin B6 |
| 783 | P35241 | RADI | Radixin |
| 784 | P35250 | RFC2 | Replication factor C subunit 2 |
| 785 | P35268 | RL22 | 60S ribosomal protein L22 |
| 786 | P35269 | T2FA | General transcription factor IIF subunit 1 |
| 787 | P35270 | SPRE | Sepiapterin reductase |
| 788 | P35527 | K1C9 | Keratin, type I cytoskeletal 9 |
| 789 | P35555 | FBN1 | Fibrillin-1 |
| 790 | P35556 | FBN2 | Fibrillin-2 |
| 791 | P35579 | MYH9 | Myosin-9 |
| 792 | P35580 | MYH10 | Myosin-10 |
| 793 | P35606 | COPB2 | Coatomer subunit beta′ |
| 794 | P35611 | ADDA | Alpha-adducin |
| 795 | P35613 | BASI | Basigin |
| 796 | P35637 | FUS | RNA-binding protein FUS |
| 797 | P35658 | NU214 | Nuclear pore complex protein Nup214 |
| 798 | P35659 | DEK | Protein DEK |
| 799 | P35749 | MYH11 | Myosin-11 |
| 800 | P35754 | GLRX1 | Glutaredoxin-1 |
| 801 | P35908 | K22E | Keratin, type II cytoskeletal 2 epidermal |
| 802 | P35998 | PRS7 | 26S protease regulatory subunit 7 |
| 803 | P36269 | GGT5 | Gamma-glutamyltransferase 5; Gamma-glutamyltransferase 5 heavy |
| | | | chain; Gamma-glutamyltransferase 5 light chain |
| 804 | P36405 | ARL3 | ADP-ribosylation factor-like protein 3 |
| 805 | P36507 | MP2K2 | Dual specificity mitogen-activated protein kinase kinase 2 |
| 806 | P36542 | ATPG | ATP synthase subunit gamma, mitochondrial |
| 807 | P36578 | RL4 | 60S ribosomal protein L4 |
| 808 | P36776 | LONM | Lon protease homolog, mitochondrial |
| 809 | P36871 | PGM1 | Phosphoglucomutase-1 |
| 810 | P36957 | ODO2 | Dihydrolipoyllysine-residue succinyltransferase component of 2- |
| | | | oxoglutarate dehydrogenase complex, mitochondrial |
| 811 | P37108 | SRP14 | Signal recognition particle 14 kDa protein |
| 812 | P37198 | NUP62 | Nuclear pore glycoprotein p62 |
| 813 | P37235 | HPCL1 | Hippocalcin-like protein 1 |
| 814 | P37802 | TAGL2 | Transgelin-2 |
| 815 | P37837 | TALDO | Transaldolase |
| 816 | P37840 | SYUA | Alpha-synuclein |
| 817 | P38117 | ETFB | Electron transfer flavoprotein subunit beta |
| 818 | P38159 | RBMX | RNA-binding motif protein, X chromosome; RNA-binding motif protein, X |
| | | | chromosome, N-terminally processed |
| 819 | P38606 | VATA | V-type proton ATPase catalytic subunit A |
| 820 | P38646 | GRP75 | Stress-70 protein, mitochondrial |
| 821 | P38919 | IF4A3 | Eukaryotic initiation factor 4A-III; Eukaryotic initiation factor 4A-III, |
| | | | N-terminally processed |
| 822 | P39019 | RS19 | 40S ribosomal protein S19 |
| 823 | P39023 | RL3 | 60S ribosomal protein L3 |
| 824 | P39059 | COFA1 | Collagen alpha-1(XV) chain; Restin; Restin-2; Restin-3; Restin-4 |
| 825 | P39060 | COIA1 | Collagen alpha-1(XVIII) chain; Endostatin |
| 826 | P39656 | OST48 | Dolichyl-diphosphooligosaccharide-protein glycosyltransferase 48 kDa |
| | | | subunit |
| 827 | P39687 | AN32A | Acidic leucine-rich nuclear phosphoprotein 32 family member A |
| 828 | P39880 | CUX1 | Homeobox protein cut-like 1 |
| 829 | P40121 | CAPG | Macrophage-capping protein |
| 830 | P40222 | TXLNA | Alpha-taxilin |
| 831 | P40227 | TCPZ | T-complex protein 1 subunit zeta |
| 832 | P40313 | CTRL | Chymotrypsin-like protease CTRL-1 |
| 833 | P40429 | RL13A | 60S ribosomal protein L13a |
| 834 | P40855 | PEX19 | Peroxisomal biogenesis factor 19 |
| 835 | P40925 | MDHC | Malate dehydrogenase, cytoplasmic |
| 836 | P40926 | MDHM | Malate dehydrogenase, mitochondrial |
| 837 | P40939 | ECHA | Long chain 3-hydroxyacyl-CoA dehydrogenase; Long-chain enoyl-CoA |
| | | | hydratase |
| 838 | P41091 | IF2G | Eukaryotic translation initiation factor 2 subunit 3 |
| 839 | P41214 | EIF2D | Eukaryotic translation initiation factor 2D |
| 840 | P41219 | PERI | Peripherin |
| 841 | P41227 | NAA10 | N-alpha-acetyltransferase 10 |
| 842 | P41236 | IPP2 | Protein phosphatase inhibitor 2 |
| 843 | P41250 | SYG | Glycine-tRNA ligase |
| 844 | P41567 | EIF1 | Eukaryotic translation initiation factor 1 |
| 845 | P42126 | ECU | Enoyl-CoA delta isomerase 1, mitochondrial |
| 846 | P42166 | LAP2A | Lamina-associated polypeptide 2, isoform |
| | | | alpha; Thymopentin; Thymopoietin |
| 847 | P42167 | LAP2B | Lamina-associated polypeptide 2, isoforms |
| | | | beta/gamma; Thymopentin; Thymopoietin |
| 848 | P42285 | SK2L2 | Superkiller viralicidic activity 2-like 2 |
| 849 | P42330 | AK1C3 | Aldo-keto reductase family 1 member C3 |
| 850 | P42345 | MTOR | Serine/threonine-protein kinase mTOR |
| 851 | P42566 | EPS15 | Epidermal growth factor receptor substrate 15 |
| 852 | P42677 | RS27 | 40S ribosomal protein S27 |
| 853 | P42704 | LPPRC | Leucine-rich PPR motif-containing protein, mitochondrial |
| 854 | P42765 | THIM | 3-ketoacyl-CoA thiolase, mitochondrial |
| 855 | P42766 | RL35 | 60S ribosomal protein L35 |
| 856 | P43034 | LIS1 | Platelet-activating factor acetylhydrolase IB subunit alpha |
| 857 | P43121 | MUC18 | Cell surface glycoprotein MUC18 |
| 858 | P43243 | MATR3 | Matrin-3 |
| 859 | P43304 | GPDM | Glycerol-3-phosphate dehydrogenase, mitochondrial |
| 860 | P43307 | SSRA | Translocon-associated protein subunit alpha |
| 861 | P43487 | RANG | Ran-specific GTPase-activating protein |
| 862 | P43686 | PRS6B | 26S protease regulatory subunit 6B |
| 863 | P43694 | GATA4 | Transcription factor GATA-4 |
| 864 | P45880 | VDAC2 | Voltage-dependent anion-selective channel protein 2 |
| 865 | P45954 | ACDSB | Short/branched chain specific acyl-CoA dehydrogenase, mitochondrial |
| 866 | P45973 | CBX5 | Chromobox protein homolog 5 |
| 867 | P45974 | UBP5 | Ubiquitin carboxyl-terminal hydrolase 5 |
| 868 | P46060 | RAGP1 | Ran GTPase-activating protein 1 |
| 869 | P46108 | CRK | Adapter molecule crk |
| 870 | P46109 | CRKL | Crk-like protein |
| 871 | P46379 | BAG6 | Large proline-rich protein BAG6 |
| 872 | P46459 | NSF | Vesicle-fusing ATPase |
| 873 | P46527 | CDN1B | Cyclin-dependent kinase inhibitor 1B |
| 874 | P46776 | RL27A | 60S ribosomal protein L27a |
| 875 | P46777 | RL5 | 60S ribosomal protein L5 |
| 876 | P46778 | RL21 | 60S ribosomal protein L21 |
| 877 | P46779 | RL28 | 60S ribosomal protein L28 |
| 878 | P46781 | RS9 | 40S ribosomal protein S9 |
| 879 | P46782 | RS5 | 40S ribosomal protein S5; 40S ribosomal protein S5, N-terminally |
| | | | processed |
| 880 | P46783 | RS10 | 40S ribosomal protein S10 |
| 881 | P46821 | MAP1B | MAP1 light chain LC1; MAP1B heavy chain; Microtubule-associated |
| | | | protein 1B |
| 882 | P46937 | YAP1 | Transcriptional coactivator YAP1 |
| 883 | P46939 | UTRO | Utrophin |
| 884 | P46940 | IQGA1 | Ras GTPase-activating-like protein IQGAP1 |
| 885 | P46976 | GLYG | Glycogenin-1 |
| 886 | P47736 | RPGP1 | Rap1 GTPase-activating protein 1 |
| 887 | P47756 | CAPZB | F-actin-capping protein subunit beta |
| 888 | P47813 | IF1AX | Eukaryotic translation initiation factor 1A, X-chromosomal |
| 889 | P47897 | SYQ | Glutamine--tRNA ligase |
| 890 | P47914 | RL29 | 60S ribosomal protein L29 |
| 891 | P47974 | TISD | Zinc finger protein 36, C3H1 type-like 2 |
| 892 | P48047 | ATPO | ATP synthase subunit O, mitochondrial |
| 893 | P48052 | CBPA2 | Carboxypeptidase A2 |
| 894 | P48061 | SDF1 | SDF-1-alpha(3-67); SDF-1-beta(3-72); Stromal cell-derived factor 1 |
| 895 | P48167 | GLRB | Glycine receptor subunit beta |
| 896 | P48304 | REG1B | Lithostathine-1-beta |
| 897 | P48444 | COPD | Coatomer subunit delta |
| 898 | P48634 | PRC2A | Protein PRRC2A |
| 899 | P48643 | TCPE | T-complex protein 1 subunit epsilon |
| 900 | P48681 | NEST | Nestin |
| 901 | P48723 | HSP13 | Heat shock 70 kDa protein 13 |
| 902 | P48735 | IDHP | Isocitrate dehydrogenase [NADP], mitochondrial |
| 903 | P48739 | PIPNB | Phosphatidylinositol transfer protein beta isoform |
| 904 | P49006 | MRP | MARCKS-related protein |
| 905 | P49023 | PAXI | Paxillin |
| 906 | P49189 | AL9A1 | 4-trimethylaminobutyraldehyde dehydrogenase |
| 907 | P49207 | RL34 | 60S ribosomal protein L34 |
| 908 | P49257 | LMAN1 | Protein ERGIC-53 |
| 909 | P49321 | NASP | Nuclear autoantigenic sperm protein |
| 910 | P49327 | FAS | [Acyl-carrier-protein] S-acetyltransferase; [Acyl-carrier-protein] |
| | | | S-malonyltransferase |
| 911 | P49368 | TCPG | T-complex protein 1 subunit gamma |
| 912 | P49407 | ARRB1 | Beta-arrestin-1 |
| 913 | P49411 | EFTU | Elongation factor Tu, mitochondrial |
| 914 | P49419 | AL7A1 | Alpha-aminoadipic semialdehyde dehydrogenase |
| 915 | P49458 | SRP09 | Signal recognition particle 9 kDa protein |
| 916 | P49588 | SYAC | Alanine-tRNA ligase, cytoplasmic |
| 917 | P49591 | SYSC | Serine-tRNA ligase, cytoplasmic |
| 918 | P49720 | PSB3 | Proteasome subunit beta type-3 |
| 919 | P49748 | ACADV | Very long-chain specific acyl-CoA dehydrogenase, mitochondrial |
| 920 | P49750 | YLPM1 | YLP motif-containing protein 1 |
| 921 | P49755 | TMEDA | Transmembrane emp24 domain-containing protein 10 |
| 922 | P49756 | RBM25 | RNA-binding protein 25 |
| 923 | P49757 | NUMB | Protein numb homolog |
| 924 | P49770 | EI2BB | Translation initiation factor elF-2B subunit beta |
| 925 | P49773 | HINT1 | Histidine triad nucleotide-binding protein 1 |
| 926 | P49790 | NU153 | Nuclear pore complex protein Nup153 |
| 927 | P49792 | RBP2 | E3 SUMO-protein ligase RanBP2 |
| 928 | P49821 | NDUV1 | NADH dehydrogenase [ubiquinone] flavoprotein 1, mitochondrial |
| 929 | P49913 | CAMP | Antibacterial protein FALL-39; Antibacterial protein LL-37; |
| | | | Cathelicidin antimicrobial peptide |
| 930 | P50135 | HNMT | Histamine N-methyltransferase |
| 931 | P50213 | IDH3A | Isocitrate dehydrogenase [NAD] subunit alpha, mitochondrial |
| 932 | P50238 | CRIP1 | Cysteine-rich protein 1 |
| 933 | P50395 | GDIB | Rab GDP dissociation inhibitor beta |
| 934 | P50402 | EMD | Emerin |
| 935 | P50440 | GATM | Glycine amidinotransferase, mitochondrial |
| 936 | P50454 | SERPH | Serpin H1 |
| 937 | P50479 | PDLI4 | PDZ and LIM domain protein 4 |
| 938 | P50552 | VASP | Vasodilator-stimulated phosphoprotein |
| 939 | P50579 | MAP2 | Methionine aminopeptidase 2 |
| 940 | P50851 | LRBA | Lipopolysaccharide-responsive and beige-like anchor protein |
| 941 | P50895 | BCAM | Basal cell adhesion molecule |
| 942 | P50897 | PPT1 | Palmitoyl-protein thioesterase 1 |
| 943 | P50914 | RL14 | 60S ribosomal protein L14 |
| 944 | P50990 | TCPQ | T-complex protein 1 subunit theta |
| 945 | P50991 | TCPD | T-complex protein 1 subunit delta |
| 946 | P50995 | ANX11 | Annexin A11 |
| 947 | P51003 | PAPOA | Poly(A) polymerase alpha |
| 948 | P51397 | DAP1 | Death-associated protein 1 |
| 949 | P51452 | DUS3 | Dual specificity protein phosphatase 3 |
| 950 | P51570 | GALK1 | Galactokinase |
| 951 | P51571 | SSRD | Translocon-associated protein subunit delta |
| 952 | P51572 | BAP31 | B-cell receptor-associated protein 31 |
| 953 | P51608 | MECP2 | Methyl-CpG-binding protein 2 |
| 954 | P51610 | HCFC1 | HCF C-terminal chain 1; HCF C-terminal chain 2; HCF C-terminal chain 3 |
| 955 | P51649 | SSDH | Succinate-semialdehyde dehydrogenase, mitochondrial |
| 956 | P51689 | ARSD | Arylsulfatase D |
| 957 | P51858 | HDGF | Hepatoma-derived growth factor |
| 958 | P51884 | LUM | Lumican |
| 959 | P51888 | PRELP | Prolargin |
| 960 | P51911 | CNN1 | Calponin-1 |
| 961 | P51970 | NDUA8 | NADH dehydrogenase [ubiquinone] 1 alpha subcomplex subunit 8 |
| 962 | P51991 | ROA3 | Heterogeneous nuclear ribonucleoprotein A3 |
| 963 | P52209 | 6PGD | 6-phosphogluconate dehydrogenase, decarboxylating |
| 964 | P52272 | HNRPM | Heterogeneous nuclear ribonucleoprotein M |
| 965 | P52565 | GDIR1 | Rho GDP-dissociation inhibitor 1 |
| 966 | P52566 | GDIR2 | Rho GDP-dissociation inhibitor 2 |
| 967 | P52594 | AGFG1 | Arf-GAP domain and FG repeat-containing protein 1 |
| 968 | P52597 | HNRPF | Heterogeneous nuclear ribonucleoprotein F; Heterogeneous nuclear |
| | | | ribonucleoprotein F, N-terminally processed |
| 969 | P52655 | TF2AA | Transcription initiation factor IIA alpha chain; Transcription |
| | | | initiation factor IIA beta chain |
| 970 | P52758 | UK114 | Ribonuclease UK114 |
| 971 | P52815 | RM12 | 39S ribosomal protein L12, mitochondrial |
| 972 | P52907 | CAZA1 | F-actin-capping protein subunit alpha-1 |
| 973 | P52943 | CRIP2 | Cysteine-rich protein 2 |
| 974 | P52948 | NUP98 | Nuclear pore complex protein Nup96; Nuclear pore complex protein |
| | | | Nup98 |
| 975 | P53007 | TXTP | Tricarboxylate transport protein, mitochondrial |
| 976 | P53367 | ARFP1 | Arfaptin-1 |
| 977 | P53384 | NUBP1 | Cytosolic Fe-S cluster assembly factor NUBP1 |
| 978 | P53396 | ACLY | ATP-citrate synthase |
| 979 | P53582 | MAP11 | Methionine aminopeptidase 1 |
| 980 | P53597 | SUCA | Succinyl-CoA ligase [ADP/GDP-forming] subunit alpha, mitochondrial |
| 981 | P53618 | COPB | Coatomer subunit beta |
| 982 | P53621 | COPA | Coatomer subunit alpha; Proxenin; Xenin |
| 983 | P53803 | RPAB4 | DNA-directed RNA polymerases I, II, and III subunit RPABC4 |
| 984 | P53814 | SMTN | Smoothelin |
| 985 | P53990 | IST1 | IST1 homolog |
| 986 | P53992 | SC24C | Protein transport protein Sec24C |
| 987 | P53999 | TCP4 | Activated RNA polymerase II transcriptional coactivator p15 |
| 988 | P54105 | ICLN | Methylosome subunit plCln |
| 989 | P54136 | SYRC | Arginine--tRNA ligase, cytoplasmic |
| 990 | P54315 | LIPR1 | Inactive pancreatic lipase-related protein 1 |
| 991 | P54317 | LIPR2 | Pancreatic lipase-related protein 2 |
| 992 | P54577 | SYYC | Tyrosine-tRNA ligase, cytoplasmic; Tyrosine-tRNA ligase, cytoplasmic, |
| | | | N-terminally processed |
| 993 | P54578 | UBP14 | Ubiquitin carboxyl-terminal hydrolase 14 |
| 994 | P54652 | HSP72 | Heat shock-related 70 kDa protein 2 |
| 995 | P54687 | BCAT1 | Branched-chain-amino-acid aminotransferase, cytosolic |
| 996 | P54725 | RD23A | UV excision repair protein RAD23 homolog A |
| 997 | P54727 | RD23B | UV excision repair protein RAD23 homolog B |
| 998 | P54819 | KAD2 | Adenylate kinase 2, mitochondrial; Adenylate kinase 2, mitochondrial, |
| | | | N-terminally processed |
| 999 | P54868 | HMCS2 | Hydroxymethylglutaryl-CoA synthase, mitochondrial |
| 1000 | P54886 | P5CS | Delta-1-pyrroline-5-carboxylate synthase; Gamma-glutamyl phosphate |
| | | | reductase; Glutamate 5-kinase |
| 1001 | P55008 | AIF1 | Allograft inflammatory factor 1 |
| 1002 | P55010 | IF5 | Eukaryotic translation initiation factor 5 |
| 1003 | P55011 | S12A2 | Solute carrier family 12 member 2 |
| 1004 | P55036 | PSMD4 | 26S proteasome non-ATPase regulatory subunit 4 |
| 1005 | P55072 | TERA | Transitional endoplasmic reticulum ATPase |
| 1006 | P55081 | MFAP1 | Microfibrillar-associated protein 1 |
| 1007 | P55084 | ECHB | 3-ketoacyl-CoA thiolase; Trifunctional enzyme subunit beta, mitochondrial |
| 1008 | P55145 | MANF | Mesencephalic astrocyte-derived neurotrophic factor |
| 1009 | P55196 | AFAD | Afadin |
| 1010 | P55209 | NP1L1 | Nucleosome assembly protein 1-like 1 |
| 1011 | P55259 | GP2 | Pancreatic secretory granule membrane major glycoprotein GP2 |
| 1012 | P55263 | ADK | Adenosine kinase |
| 1013 | P55265 | DSRAD | Double-stranded RNA-specific adenosine deaminase |
| 1014 | P55268 | LAMB2 | Laminin subunit beta-2 |
| 1015 | P55327 | TPD52 | Tumor protein D52 |
| 1016 | P55735 | SEC13 | Protein SEC13 homolog |
| 1017 | P55769 | NH2L1 | NHP2-like protein 1; NHP2-like protein 1, N-terminally processed |
| 1018 | P55884 | EIF3B | Eukaryotic translation initiation factor 3 subunit B |
| 1019 | P56134 | ATPK | ATP synthase subunit f, mitochondrial |
| 1020 | P56192 | SYMC | Methionine-tRNA ligase, cytoplasmic |
| 1021 | P56199 | ITA1 | Integrin alpha-1 |
| 1022 | P56211 | ARP19 | cAMP-regulated phosphoprotein 19 |
| 1023 | P56277 | CMC4 | Cx9C motif-containing protein 4 |
| 1024 | P56381 | ATP5E | ATP synthase subunit epsilon, mitochondrial |
| 1025 | P56385 | ATP5I | ATP synthase subunit e, mitochondrial |
| 1026 | P56537 | IF6 | Eukaryotic translation initiation factor 6 |
| 1027 | P56645 | PER3 | Period circadian protein homolog 3 |
| 1028 | P57081 | WDR4 | tRNA (guanine-N(7)-)-methyltransferase non-catalytic subunit WDR4 |
| 1029 | P57737 | CORO7 | Coronin-7 |
| 1030 | P58107 | EPIPL | Epiplakin |
| 1031 | P58546 | MTPN | Myotrophin |
| 1032 | P59665 | DEF1 | HP 1-56; Neutrophil defensin 1 ; Neutrophil defensin 2 |
| 1033 | P59998 | ARPC4 | Actin-related protein 2/3 complex subunit 4 |
| 1034 | P60002 | ELOF1 | Transcription elongation factor 1 homolog |
| 1035 | P60006 | APC15 | Anaphase-promoting complex subunit 15 |
| 1036 | P60022 | DEFB1 | Beta-defensin 1 |
| 1037 | P60059 | SC61G | Protein transport protein Sec61 subunit gamma |
| 1038 | P60174 | TPIS | Triosephosphate isomerase |
| 1039 | P60468 | SC61B | Protein transport protein Sec61 subunit beta |
| 1040 | P60520 | GBRL2 | Gamma-aminobutyric acid receptor-associated protein-like 2 |
| 1041 | P60660 | MYL6 | Myosin light polypeptide 6 |
| 1042 | P60709 | ACTB | Actin, cytoplasmic 1; Actin, cytoplasmic 1, N-terminally processed |
| 1043 | P60842 | IF4A1 | Eukaryotic initiation factor 4A-I |
| 1044 | P60866 | RS20 | 40S ribosomal protein S20 |
| 1045 | P60900 | PSA6 | Proteasome subunit alpha type-6 |
| 1046 | P60903 | S10AA | Protein S100-A10 |
| 1047 | P60953 | CDC42 | Cell division control protein 42 homolog |
| 1048 | P60981 | DEST | Destrin |
| 1049 | P60983 | GMFB | Glia maturation factor beta |
| 1050 | P61009 | SPCS3 | Signal peptidase complex subunit 3 |
| 1051 | P61011 | SRP54 | Signal recognition particle 54 kDa protein |
| 1052 | P61019 | RAB2A | Ras-related protein Rab-2A |
| 1053 | P61026 | RAB10 | Ras-related protein Rab-10 |
| 1054 | P61081 | UBC12 | NEDD8-conjugating enzyme Ubc12 |
| 1055 | P61088 | UBE2N | Ubiquitin-conjugating enzyme E2 N |
| 1056 | P61158 | ARP3 | Actin-related protein 3 |
| 1057 | P61160 | ARP2 | Actin-related protein 2 |
| 1058 | P61163 | ACTZ | Alpha-centractin |
| 1059 | P61204 | ARF3 | ADP-ribosylation factor 3 |
| 1060 | P61221 | ABCE1 | ATP-binding cassette sub-family E member 1 |
| 1061 | P61244 | MAX | Protein max |
| 1062 | P61247 | RS3A | 40S ribosomal protein S3a |
| 1063 | P61278 | SMS | Somatostatin; Somatostatin-14; Somatostatin-28 |
| 1064 | P61313 | RL15 | 60S ribosomal protein L15 |
| 1065 | P61353 | RL27 | 60S ribosomal protein L27 |
| 1066 | P61457 | PHS | Pterin-4-alpha-carbinolamine dehydratase |
| 1067 | P61513 | RL37A | 60S ribosomal protein L37a |
| 1068 | P61604 | CH10 | 10 kDa heat shock protein, mitochondrial |
| 1069 | P61626 | LYSC | Lysozyme C |
| 1070 | P61758 | PFD3 | Prefoldin subunit 3 |
| 1071 | P61769 | B2MG | Beta-2-microglobulin; Beta-2-microglobulin form pl 5.3 |
| 1072 | P61916 | NPC2 | Epididymal secretory protein E1 |
| 1073 | P61956 | SUMO2 | Small ubiquitin-related modifier 2 |
| 1074 | P61960 | UFM1 | Ubiquitin-fold modifier 1 |
| 1075 | P61964 | WDR5 | WD repeat-containing protein 5 |
| 1076 | P61978 | HNRPK | Heterogeneous nuclear ribonucleoprotein K |
| 1077 | P61981 | 1433G | 14-3-3 protein gamma; 14-3-3 protein gamma, N-terminally processed |
| 1078 | P62072 | TIM10 | Mitochondrial import inner membrane translocase subunit Tim10 |
| 1079 | P62081 | RS7 | 40S ribosomal protein S7 |
| 1080 | P62136 | PP1A | Serine/threonine-protein phosphatase PP1-alpha catalytic subunit |
| 1081 | P62140 | PP1B | Serine/threonine-protein phosphatase PP1-beta catalytic subunit |
| 1082 | P62158 | CALM | Calmodulin |
| 1083 | P62191 | PRS4 | 26S protease regulatory subunit 4 |
| 1084 | P62195 | PRS8 | 26S protease regulatory subunit 8 |
| 1085 | P62241 | RS8 | 40S ribosomal protein S8 |
| 1086 | P62244 | RS15A | 40S ribosomal protein S15a |
| 1087 | P62249 | RS16 | 40S ribosomal protein S16 |
| 1088 | P62258 | 1433E | 14-3-3 protein epsilon |
| 1089 | P62263 | RS14 | 40S ribosomal protein S14 |
| 1090 | P62266 | RS23 | 40S ribosomal protein S23 |
| 1091 | P62269 | RS18 | 40S ribosomal protein S18 |
| 1092 | P62273 | RS29 | 40S ribosomal protein S29 |
| 1093 | P62277 | RS13 | 40S ribosomal protein S13 |
| 1094 | P62280 | RS11 | 40S ribosomal protein S11 |
| 1095 | P62306 | RUXF | Small nuclear ribonucleoprotein F |
| 1096 | P62310 | LSM3 | U6 snRNA-associated Sm-like protein LSm3 |
| 1097 | P62312 | LSM6 | U6 snRNA-associated Sm-like protein LSm6 |
| 1098 | P62314 | SMD1 | Small nuclear ribonucleoprotein Sm D1 |
| 1099 | P62316 | SMD2 | Small nuclear ribonucleoprotein Sm D2 |
| 1100 | P62318 | SMD3 | Small nuclear ribonucleoprotein Sm D3 |
| 1101 | P62328 | TYB4 | Hematopoietic system regulatory peptide; Thymosin beta-4 |
| 1102 | P62333 | PRS10 | 26S protease regulatory subunit 10B |
| 1103 | P62424 | RL7A | 60S ribosomal protein L7a |
| 1104 | P62633 | CNBP | Cellular nucleic acid-binding protein |
| 1105 | P62701 | RS4X | 40S ribosomal protein S4, X isoform |
| 1106 | P62750 | RL23A | 60S ribosomal protein L23a |
| 1107 | P62753 | RS6 | 40S ribosomal protein S6 |
| 1108 | P62805 | H4 | Histone H4 |
| 1109 | P62826 | RAN | GTP-binding nuclear protein Ran |
| 1110 | P62829 | RL23 | 60S ribosomal protein L23 |
| 1111 | P62841 | RS15 | 40S ribosomal protein S15 |
| 1112 | P62847 | RS24 | 40S ribosomal protein S24 |
| 1113 | P62851 | RS25 | 40S ribosomal protein S25 |
| 1114 | P62854 | RS26 | 40S ribosomal protein S26 |
| 1115 | P62857 | RS28 | 40S ribosomal protein S28 |
| 1116 | P62861 | RS30 | 40S ribosomal protein S30 |
| 1117 | P62873 | GBB1 | Guanine nucleotide-binding protein G(I)/G(S)/G(T) subunit beta-1 |
| 1118 | P62879 | GBB2 | Guanine nucleotide-binding protein G(I)/G(S)/G(T) subunit beta-2 |
| 1119 | P62888 | RL30 | 60S ribosomal protein L30 |
| 1120 | P62891 | RL39 | 60S ribosomal protein L39 |
| 1121 | P62899 | RL31 | 60S ribosomal protein L31 |
| 1122 | P62906 | RL10A | 60S ribosomal protein L10a |
| 1123 | P62910 | RL32 | 60S ribosomal protein L32 |
| 1124 | P62913 | RL11 | 60S ribosomal protein L11 |
| 1125 | P62917 | RL8 | 60S ribosomal protein L8 |
| 1126 | P62937 | PPIA | Peptidyl-prolyl cis-trans isomerase A; Peptidyl-prolyl cis-trans |
| | | | isomerase A, N-terminally processed |
| 1127 | P62942 | FKB1A | Peptidyl-prolyl cis-trans isomerase FKBP1A |
| 1128 | P62979 | RS27A | 40S ribosomal protein S27a; Ubiquitin; Ubiquitin-40S ribosomal |
| | | | protein S27a |
| 1129 | P62993 | GRB2 | Growth factor receptor-bound protein 2 |
| 1130 | P62995 | TRA2B | Transformer-2 protein homolog beta |
| 1131 | P63027 | VAMP2 | Vesicle-associated membrane protein 2 |
| 1132 | P63104 | 1433Z | 14-3-3 protein zeta/delta |
| 1133 | P63151 | 2ABA | Serine/threonine-protein phosphatase 2A 55 kDa regulatory |
| | | | subunit B alpha isoform |
| 1134 | P63165 | SUMO1 | Small ubiquitin-related modifier 1 |
| 1135 | P63173 | RL38 | 60S ribosomal protein L38 |
| 1136 | P63208 | SKP1 | S-phase kinase-associated protein 1 |
| 1137 | P63220 | RS21 | 40S ribosomal protein S21 |
| 1138 | P63241 | IF5A1 | Eukaryotic translation initiation factor 5A-1 |
| 1139 | P63244 | RACK1 | Guanine nucleotide-binding protein subunit beta-2-like 1 |
| 1140 | P63261 | ACTG | Actin, cytoplasmic 2; Actin, cytoplasmic 2, N-terminally processed |
| 1141 | P63267 | ACTH | Actin, gamma-enteric smooth muscle |
| 1142 | P63313 | TYB10 | Thymosin beta-10 |
| 1143 | P67809 | YBOX1 | Nuclease-sensitive element-binding protein 1 |
| 1144 | P67936 | TPM4 | Tropomyosin alpha-4 chain |
| 1145 | P68036 | UB2L3 | Ubiquitin-conjugating enzyme E2 L3 |
| 1146 | P68106 | FKB1B | Peptidyl-prolyl cis-trans isomerase FKBP1B |
| 1147 | P68366 | TBA4A | Tubulin alpha-4A chain |
| 1148 | P68371 | TBB4B | Tubulin beta-4B chain |
| 1149 | P68402 | PA1B2 | Platelet-activating factor acetylhydrolase IB subunit beta |
| 1150 | P68871 | HBB | Hemoglobin subunit beta; LW-hemorphin-7; Spinorphin |
| 1151 | P69905 | HBA | Hemoglobin subunit alpha |
| 1152 | P78310 | CXAR | Coxsackievirus and adenovirus receptor |
| 1153 | P78329 | CP4F2 | Phylloquinone omega-hydroxylase CYP4F2 |
| 1154 | P78371 | TCPB | T-complex protein 1 subunit beta |
| 1155 | P78406 | RAE1L | mRNA export factor |
| 1156 | P78417 | GSTO1 | Glutathione S-transferase omega-1 |
| 1157 | P80188 | NGAL | Neutrophil gelatinase-associated lipocalin |
| 1158 | P80297 | MT1X | Metallothionein-1X |
| 1159 | P80303 | NUCB2 | Nesfatin-1; Nucleobindin-2 |
| 1160 | P80404 | GABT | 4-aminobutyrate aminotransferase, mitochondrial |
| 1161 | P80723 | BASP1 | Brain acid soluble protein 1 |
| 1162 | P80748 | LV302 | Ig lambda chain V-III region LOI |
| 1163 | P81605 | DCD | DCD-1; Dermcidin; Survival-promoting peptide |
| 1164 | P82094 | TMF1 | TATA element modulatory factor |
| 1165 | P82673 | RT35 | 28S ribosomal protein S35, mitochondrial |
| 1166 | P82909 | RT36 | 28S ribosomal protein S36, mitochondrial |
| 1167 | P82914 | RT15 | 28S ribosomal protein S15, mitochondrial |
| 1168 | P82970 | HMGN5 | High mobility group nucleosome-binding domain-containing protein 5 |
| 1169 | P82979 | SARNP | SAP domain-containing ribonucleoprotein |
| 1170 | P83731 | RL24 | 60S ribosomal protein L24 |
| 1171 | P83916 | CBX1 | Chromobox protein homolog 1 |
| 1172 | P84090 | ERH | Enhancer of rudimentary homolog |
| 1173 | P84098 | RL19 | 60S ribosomal protein L19 |
| 1174 | P84103 | SRSF3 | Serine/arginine-rich splicing factor 3 |
| 1175 | P84243 | H33 | Histone H3.3 |
| 1176 | P98082 | DAB2 | Disabled homolog 2 |
| 1177 | P98088 | MUC5A | Mucin-5AC |
| 1178 | P98095 | FBLN2 | Fibulin-2 |
| 1179 | P98160 | PGBM | Basement membrane-specific heparan sulfate proteoglycan core |
| | | | protein; Endorepellin; LG3 peptide |
| 1180 | P98175 | RBM10 | RNA-binding protein 10 |
| 1181 | P98179 | RBM3 | RNA-binding protein 3 |
| 1182 | P99999 | CYC | Cytochrome c |
| 1183 | Q00059 | TFAM | Transcription factor A, mitochondrial |
| 1184 | Q00325 | MPCP | Phosphate carrier protein, mitochondrial |
| 1185 | Q00341 | VIGLN | Vigilin |
| 1186 | Q00403 | TF2B | Transcription initiation factor IIB |
| 1187 | Q00577 | PURA | Transcriptional activator protein Pur-alpha |
| 1188 | Q00587 | BORG5 | Cdc42 effector protein 1 |
| 1189 | Q00610 | CLH1 | Clathrin heavy chain 1 |
| 1190 | Q00688 | FKBP3 | Peptidyl-prolyl cis-trans isomerase FKBP3 |
| 1191 | Q00796 | DHSO | Sorbitol dehydrogenase |
| 1192 | Q00839 | HNRPU | Heterogeneous nuclear ribonucleoprotein U |
| 1193 | Q01082 | SPTB2 | Spectrin beta chain, non-erythrocytic 1 |
| 1194 | Q01105 | SET | Protein SET |
| 1195 | Q01130 | SRSF2 | Serine/arginine-rich splicing factor 2 |
| 1196 | Q01433 | AMPD2 | AMP deaminase 2 |
| 1197 | Q01469 | FABP5 | Fatty acid-binding protein, epidermal |
| 1198 | Q01518 | CAP1 | Adenylyl cyclase-associated protein 1 |
| 1199 | Q01658 | NC2B | Protein Dr1 |
| 1200 | Q01804 | OTUD4 | OTU domain-containing protein 4 |
| 1201 | Q01844 | EWS | RNA-binding protein EWS |
| 1202 | Q01995 | TAGL | Transgelin |
| 1203 | Q02218 | ODO1 | 2-oxoglutarate dehydrogenase, mitochondrial |
| 1204 | Q02252 | MMSA | Methylmalonate-semialdehyde dehydrogenase [acylating], mitochondrial |
| 1205 | Q02447 | SP3 | Transcription factor Sp3 |
| 1206 | Q02487 | DSC2 | Desmocollin-2 |
| 1207 | Q02543 | RL18A | 60S ribosomal protein L18a |
| 1208 | Q02790 | FKBP4 | Peptidyl-prolyl cis-trans isomerase FKBP4; Peptidyl-prolyl cis-trans |
| | | | isomerase FKBP4, N-terminally processed |
| 1209 | Q02818 | NUCB1 | Nucleobindin-1 |
| 1210 | Q02878 | RL6 | 60S ribosomal protein L6 |
| 1211 | Q02952 | AKA12 | A-kinase anchor protein 12 |
| 1212 | Q03188 | CENPC | Centromere protein C |
| 1213 | Q03252 | LMNB2 | Lamin-B2 |
| 1214 | Q03403 | TFF2 | Trefoil factor 2 |
| 1215 | Q04323 | UBXN1 | UBX domain-containing protein 1 |
| 1216 | Q04637 | IF4G1 | Eukaryotic translation initiation factor 4 gamma 1 |
| 1217 | Q04760 | LGUL | Lactoylglutathione lyase |
| 1218 | Q04837 | SSBP | Single-stranded DNA-binding protein, mitochondrial |
| 1219 | Q04917 | 1433F | 14-3-3 protein eta |
| 1220 | Q05084 | ICA69 | Islet cell autoantigen 1 |
| 1221 | Q05209 | PTN12 | Tyrosine-protein phosphatase non-receptor type 12 |
| 1222 | Q05519 | SRS11 | Serine/arginine-rich splicing factor 11 |
| 1223 | Q05639 | EF1A2 | Elongation factor 1-alpha 2 |
| 1224 | Q05682 | CALD1 | Caldesmon |
| 1225 | Q05707 | COEA1 | Collagen alpha-1(XIV) chain |
| 1226 | Q06141 | REG3A | Regenerating islet-derived protein 3-alpha; Regenerating islet-derived |
| | | | protein 3-alpha 15 kDa form |
| 1227 | Q06210 | GFPT1 | Glutamine-fructose-6-phosphate aminotransferase [isomerizing] 1 |
| 1228 | Q06278 | AOXA | Aldehyde oxidase |
| 1229 | Q06323 | PSME1 | Proteasome activator complex subunit 1 |
| 1230 | Q06546 | GABPA | GA-binding protein alpha chain |
| 1231 | Q06830 | PRDX1 | Peroxiredoxin-1 |
| 1232 | Q07020 | RL18 | 60S ribosomal protein L18 |
| 1233 | Q07021 | C1QBP | Complement component 1 Q subcomponent-binding protein, |
| | | | mitochondrial |
| 1234 | Q07065 | CKAP4 | Cytoskeleton-associated protein 4 |
| 1235 | Q07092 | COGA1 | Collagen alpha-1(XVI) chain |
| 1236 | Q07157 | ZO1 | Tight junction protein ZO-1 |
| 1237 | Q07666 | KHDR1 | KH domain-containing, RNA-binding, signal transduction-associated |
| | | | protein 1 |
| 1238 | Q07866 | KLC1 | Kinesin light chain 1 |
| 1239 | Q07954 | LRP1 | Low-density lipoprotein receptor-related protein 1 515 kDa subunit |
| 1240 | Q07955 | SRSF1 | Serine/arginine-rich splicing factor 1 |
| 1241 | Q07960 | RHG01 | Rho GTPase-activating protein 1 |
| 1242 | Q08043 | ACTN3 | Alpha-actinin-3 |
| 1243 | Q08170 | SRSF4 | Serine/arginine-rich splicing factor 4 |
| 1244 | Q08211 | DHX9 | ATP-dependent RNA helicase A |
| 1245 | Q08257 | QOR | Quinone oxidoreductase |
| 1246 | Q08378 | GOGA3 | Golgin subfamily A member 3 |
| 1247 | Q08379 | GOGA2 | Golgin subfamily A member 2 |
| 1248 | Q08380 | LG3BP | Galectin-3-binding protein |
| 1249 | Q08397 | LOXL1 | Lysyl oxidase homolog 1 |
| 1250 | Q08431 | MFGM | Lactadherin; Lactadherin short form; Medin |
| 1251 | Q08495 | DEMA | Dematin |
| 1252 | Q08752 | PPID | Peptidyl-prolyl cis-trans isomerase D |
| 1253 | Q08945 | SSRP1 | FACT complex subunit SSRP1 |
| 1254 | Q09028 | RBBP4 | Histone-binding protein RBBP4 |
| 1255 | Q09666 | AHNK | Neuroblast differentiation-associated protein AHNAK |
| 1256 | Q0VAF6 | SYCN | Syncoilin |
| 1257 | Q0VF96 | CGNL1 | Cingulin-like protein 1 |
| 1258 | Q0VGL1 | LTOR4 | Ragulator complex protein LAMTOR4; Ragulator complex protein |
| | | | LAMTOR4, N-terminally processed |
| 1259 | Q0ZGT2 | NEXN | Nexilin |
| 1260 | Q10567 | AP1B1 | AP-1 complex subunit beta-1 |
| 1261 | Q12765 | SCRN1 | Secernin-1 |
| 1262 | Q12805 | FBLN3 | EGF-containing fibulin-like extracellular matrix protein 1 |
| 1263 | Q12841 | FSTL1 | Follistatin-related protein 1 |
| 1264 | Q12874 | SF3A3 | Splicing factor 3A subunit 3 |
| 1265 | Q12888 | TP53B | Tumor suppressor p53-binding protein 1 |
| 1266 | Q12904 | AIMP1 | Aminoacyl tRNA synthase complex-interacting multifunctional protein 1 |
| 1267 | Q12906 | ILF3 | Interleukin enhancer-binding factor 3 |
| 1268 | Q12907 | LMAN2 | Vesicular integral-membrane protein VIP36 |
| 1269 | Q12912 | LRMP | Lymphoid-restricted membrane protein; Processed lymphoid-restricted |
| | | | membrane protein |
| 1270 | Q12929 | EPS8 | Epidermal growth factor receptor kinase substrate 8 |
| 1271 | Q12931 | TRAP1 | Heat shock protein 75 kDa, mitochondrial |
| 1272 | Q12955 | ANK3 | Ankyrin-3 |
| 1273 | Q12959 | DLG1 | Disks large homolog 1 |
| 1274 | Q13011 | ECH1 | Delta(3,5)-Delta(2,4)-dienoyl-CoA isomerase, mitochondrial |
| 1275 | Q13087 | PDIA2 | Protein disulfide-isomerase A2 |
| 1276 | Q13098 | CSN1 | COP9 signalosome complex subunit 1 |
| 1277 | Q13103 | SPP24 | Secreted phosphoprotein 24 |
| 1278 | Q13123 | RED | Protein Red |
| 1279 | Q13126 | MTAP | S-methyl-5′-thioadenosine phosphorylase |
| 1280 | Q13144 | EI2BE | Translation initiation factor elF-2B subunit epsilon |
| 1281 | Q13148 | TADBP | TAR DNA-binding protein 43 |
| 1282 | Q13151 | ROAO | Heterogeneous nuclear ribonucleoprotein A0 |
| 1283 | Q13162 | PRDX4 | Peroxiredoxin-4 |
| 1284 | Q13177 | PAK2 | PAK-2p27; PAK-2p34; Serine/threonine-protein kinase PAK 2 |
| 1285 | Q13185 | CBX3 | Chromobox protein homolog 3 |
| 1286 | Q13200 | PSMD2 | 26S proteasome non-ATPase regulatory subunit 2 |
| 1287 | Q13217 | DNJC3 | DnaJ homolog subfamily C member 3 |
| 1288 | Q13228 | SBP1 | Selenium-binding protein 1 |
| 1289 | Q13242 | SRSF9 | Serine/arginine-rich splicing factor 9 |
| 1290 | Q13243 | SRSF5 | Serine/arginine-rich splicing factor 5 |
| 1291 | Q13247 | SRSF6 | Serine/arginine-rich splicing factor 6 |
| 1292 | Q13263 | TIF1B | Transcription intermediary factor 1-beta |
| 1293 | Q13268 | DHRS2 | Dehydrogenase/reductase SDR family member 2, mitochondrial |
| 1294 | Q13283 | G3BP1 | Ras GTPase-activating protein-binding protein 1 |
| 1295 | Q13310 | PABP4 | Polyadenylate-binding protein 4 |
| 1296 | Q13347 | EIF3I | Eukaryotic translation initiation factor 3 subunit I |
| 1297 | Q13361 | MFAP5 | Microfibrillar-associated protein 5 |
| 1298 | Q13404 | UB2V1 | Ubiquitin-conjugating enzyme E2 variant 1 |
| 1299 | Q13405 | RM49 | 39S ribosomal protein L49, mitochondrial |
| 1300 | Q13409 | DC1I2 | Cytoplasmic dynein 1 intermediate chain 2 |
| 1301 | Q13423 | NNTM | NAD(P) transhydrogenase, mitochondrial |
| 1302 | Q13428 | TCOF | Treacle protein |
| 1303 | Q13435 | SF3B2 | Splicing factor 3B subunit 2 |
| 1304 | Q13438 | OS9 | Protein OS-9 |
| 1305 | Q13439 | GOGA4 | Golgin subfamily A member 4 |
| 1306 | Q13442 | HAP28 | 28 kDa heat- and acid-stable phosphoprotein |
| 1307 | Q13451 | FKBP5 | Peptidyl-prolyl cis-trans isomerase FKBP5; Peptidyl-prolyl |
| | | | cis-trans isomerase FKBP5, N-terminally processed |
| 1308 | Q13459 | MYO9B | Unconventional myosin-IXb |
| 1309 | Q13492 | PICAL | Phosphatidylinositol-binding clathrin assembly protein |
| 1310 | Q13501 | SQSTM | Sequestosome-1 |
| 1311 | Q13510 | ASAH1 | Acid ceramidase; Acid ceramidase subunit alpha; Acid ceramidase |
| | | | subunit beta |
| 1312 | Q13526 | PIN1 | Peptidyl-prolyl cis-trans isomerase NIMA-interacting 1 |
| 1313 | Q13541 | 4EBP1 | Eukaryotic translation initiation factor 4E-binding protein 1 |
| 1314 | Q13542 | 4EBP2 | Eukaryotic translation initiation factor 4E-binding protein 2 |
| 1315 | Q13557 | KCC2D | Calcium/calmodulin-dependent protein kinase type II subunit delta |
| 1316 | Q13561 | DCTN2 | Dynactin subunit 2 |
| 1317 | Q13573 | SNW1 | SNW domain-containing protein 1 |
| 1318 | Q13576 | IQGA2 | Ras GTPase-activating-like protein IQGAP2 |
| 1319 | Q13586 | STIM1 | Stromal interaction molecule 1 |
| 1320 | Q13595 | TRA2A | Transformer-2 protein homolog alpha |
| 1321 | Q13596 | SNX1 | Sorting nexin-1 |
| 1322 | Q13620 | CUL4B | Cullin-4B |
| 1323 | Q13630 | FCL | GDP-L-fucose synthase |
| 1324 | Q13642 | FHL1 | Four and a half LIM domains protein 1 |
| 1325 | Q13643 | FHL3 | Four and a half LIM domains protein 3 |
| 1326 | Q13740 | CD166 | CD166 antigen |
| 1327 | Q13796 | SHRM2 | Protein Shroom2 |
| 1328 | Q13813 | SPTN1 | Spectrin alpha chain, non-erythrocytic 1 |
| 1329 | Q13838 | DX39B | Spliceosome RNA helicase DDX39B |
| 1330 | Q13884 | SNTB1 | Beta-1-syntrophin |
| 1331 | Q13885 | TBB2A | Tubulin beta-2A chain |
| 1332 | Q13938 | CAYP1 | Calcyphosin |
| 1333 | Q14011 | CIRBP | Cold-inducible RNA-binding protein |
| 1334 | Q14019 | COTL1 | Coactosin-like protein |
| 1335 | Q14061 | COX17 | Cytochrome c oxidase copper chaperone |
| 1336 | Q14103 | HNRPD | Heterogeneous nuclear ribonucleoprotein D0 |
| 1337 | Q14112 | NID2 | Nidogen-2 |
| 1338 | Q14118 | DAG1 | Alpha-dystroglycan; Beta-dystroglycan; Dystroglycan |
| 1339 | Q14126 | DSG2 | Desmoglein-2 |
| 1340 | Q14151 | SAFB2 | Scaffold attachment factor B2 |
| 1341 | Q14152 | EIF3A | Eukaryotic translation initiation factor 3 subunit A |
| 1342 | Q14155 | ARHG7 | Rho guanine nucleotide exchange factor 7 |
| 1343 | Q14157 | UBP2L | Ubiquitin-associated protein 2-like |
| 1344 | Q14160 | SCRIB | Protein scribble homolog |
| 1345 | Q14165 | MLEC | Malectin |
| 1346 | Q14195 | DPYL3 | Dihydropyrimidinase-related protein 3 |
| 1347 | Q14197 | ICT1 | Peptidyl-tRNA hydrolase ICT1, mitochondrial |
| 1348 | Q14203 | DCTN1 | Dynactin subunit 1 |
| 1349 | Q14204 | DYHC1 | Cytoplasmic dynein 1 heavy chain 1 |
| 1350 | Q14240 | IF4A2 | Eukaryotic initiation factor 4A-II; Eukaryotic initiation |
| | | | factor 4A-II, N-terminally processed |
| 1351 | Q14244 | MAP7 | Ensconsin |
| 1352 | Q14247 | SRC8 | Src substrate cortactin |
| 1353 | Q14258 | TRI25 | E3 ubiquitin/ISG15 ligase TRIM25 |
| 1354 | Q14315 | FLNC | Filamin-C |
| 1355 | Q14353 | GAMT | Guanidinoacetate N-methyltransferase |
| 1356 | Q14432 | PDE3A | cGMP-inhibited 3′,5′-cyclic phosphodiesterase A |
| 1357 | Q14444 | CAPR1 | Caprin-1 |
| 1358 | Q14498 | RBM39 | RNA-binding protein 39 |
| 1359 | Q14554 | PDIA5 | Protein disulfide-isomerase A5 |
| 1360 | Q14671 | PUM1 | Pumilio homolog 1 |
| 1361 | Q14677 | EPN4 | Clathrin interactor 1 |
| 1362 | Q14683 | SMC1A | Structural maintenance of chromosomes protein 1A |
| 1363 | Q14696 | MESD | LDLR chaperone MESD |
| 1364 | Q14697 | GANAB | Neutral alpha-glucosidase AB |
| 1365 | Q14749 | GNMT | Glycine N-methyltransferase |
| 1366 | Q14764 | MVP | Major vault protein |
| 1367 | Q14766 | LTBP1 | Latent-transforming growth factor beta-binding protein 1 |
| 1368 | Q14767 | LTBP2 | Latent-transforming growth factor beta-binding protein 2 |
| 1369 | Q14789 | GOGB1 | Golgin subfamily B member 1 |
| 1370 | Q14847 | LASP1 | LIM and SH3 domain protein 1 |
| 1371 | Q14956 | GPNMB | Transmembrane glycoprotein NMB |
| 1372 | Q14966 | ZN638 | Zinc finger protein 638 |
| 1373 | Q14974 | IMB1 | Importin subunit beta-1 |
| 1374 | Q14978 | NOLC1 | Nucleolar and coiled-body phosphoprotein 1 |
| 1375 | Q14980 | NUMA1 | Nuclear mitotic apparatus protein 1 |
| 1376 | Q14BN4 | SLMAP | Sarcolemmal membrane-associated protein |
| 1377 | Q14C86 | GAPD1 | GTPase-activating protein and VPS9 domain-containing protein 1 |
| 1378 | Q15005 | SPCS2 | Signal peptidase complex subunit 2 |
| 1379 | Q15018 | F175B | BRISC complex subunit Abro1 |
| 1380 | Q15019 | 37500 | Septin-2 |
| 1381 | Q15046 | SYK | Lysine-tRNA ligase |
| 1382 | Q15056 | IF4H | Eukaryotic translation initiation factor 4H |
| 1383 | Q15063 | POSTN | Periostin |
| 1384 | Q15075 | EEA1 | Early endosome antigen 1 |
| 1385 | Q15084 | PDIA6 | Protein disulfide-isomerase A6 |
| 1386 | Q15121 | PEA15 | Astrocytic phosphoprotein PEA-15 |
| 1387 | Q15149 | PLEC | Plectin |
| 1388 | Q15154 | PCM1 | Pericentriolar material 1 protein |
| 1389 | Q15155 | NOMO1 | Nodal modulator 1 |
| 1390 | Q15181 | IPYR | Inorganic pyrophosphatase |
| 1391 | Q15185 | TEBP | Prostaglandin E synthase 3 |
| 1392 | Q15233 | NONO | Non-POU domain-containing octamer-binding protein |
| 1393 | Q15276 | RABE1 | Rab GTPase-binding effector protein 1 |
| 1394 | Q15287 | RNPS1 | RNA-binding protein with serine-rich domain 1 |
| 1395 | Q15293 | RCN1 | Reticulocalbin-1 |
| 1396 | Q15363 | TMED2 | Transmembrane emp24 domain-containing protein 2 |
| 1397 | Q15365 | PCBP1 | Poly(rC)-binding protein 1 |
| 1398 | Q15366 | PCBP2 | Poly(rC)-binding protein 2 |
| 1399 | Q15369 | ELOC | Transcription elongation factor B polypeptide 1 |
| 1400 | Q15370 | ELOB | Transcription elongation factor B polypeptide 2 |
| 1401 | Q15417 | CNN3 | Calponin-3 |
| 1402 | Q15424 | SAFB1 | Scaffold attachment factor B1 |
| 1403 | Q15427 | SF3B4 | Splicing factor 3B subunit 4 |
| 1404 | Q15428 | SF3A2 | Splicing factor 3A subunit 2 |
| 1405 | Q15435 | PP1R7 | Protein phosphatase 1 regulatory subunit 7 |
| 1406 | Q15437 | SC23B | Protein transport protein Sec23B |
| 1407 | Q15459 | SF3A1 | Splicing factor 3A subunit 1 |
| 1408 | Q15493 | RGN | Regucalcin |
| 1409 | Q15532 | SSXT | Protein SSXT |
| 1410 | Q15582 | BGH3 | Transforming growth factor-beta-induced protein ig-h3 |
| 1411 | Q15599 | NHRF2 | Na(+)/H(+) exchange regulatory cofactor NHE-RF2 |
| 1412 | Q15637 | SF01 | Splicing factor 1 |
| 1413 | Q15642 | CIP4 | Cdc42-interacting protein 4 |
| 1414 | Q15643 | TRIPB | Thyroid receptor-interacting protein 11 |
| 1415 | Q15651 | HMGN3 | High mobility group nucleosome-binding domain-containing protein 3 |
| 1416 | Q15691 | MARE1 | Microtubule-associated protein RP/EB family member 1 |
| 1417 | Q15717 | ELAV1 | ELAV-like protein 1 |
| 1418 | Q15746 | MYLK | Myosin light chain kinase, smooth muscle; Myosin light chain kinase, |
| | | | smooth muscle, deglutamylated form |
| 1419 | Q15819 | UB2V2 | Ubiquitin-conjugating enzyme E2 variant 2 |
| 1420 | Q15836 | VAMP3 | Vesicle-associated membrane protein 3 |
| 1421 | Q15843 | NEDD8 | NEDD8 |
| 1422 | Q15847 | ADIRF | Adipogenesis regulatory factor |
| 1423 | Q15907 | RB11B | Ras-related protein Rab-11B |
| 1424 | Q15942 | ZYX | Zyxin |
| 1425 | Q16134 | ETFD | Electron transfer flavoprotein-ubiquinone oxidoreductase, mitochondrial |
| 1426 | Q16181 | 39326 | Septin-7 |
| 1427 | Q16186 | ADRM1 | Proteasomal ubiquitin receptor ADRM1 |
| 1428 | Q16204 | CCDC6 | Coiled-coil domain-containing protein 6 |
| 1429 | Q16270 | IBP7 | Insulin-like growth factor-binding protein 7 |
| 1430 | Q16363 | LAMA4 | Laminin subunit alpha-4 |
| 1431 | Q16401 | PSMD5 | 26S proteasome non-ATPase regulatory subunit 5 |
| 1432 | Q16527 | CSRP2 | Cysteine and glycine-rich protein 2 |
| 1433 | Q16531 | DDB1 | DNA damage-binding protein 1 |
| 1434 | Q16543 | CDC37 | Hsp90 co-chaperone Cdc37; Hsp90 co-chaperone Cdc37, N-terminally |
| | | | processed |
| 1435 | Q16555 | DPYL2 | Dihydropyrimidinase-related protein 2 |
| 1436 | Q16568 | CART | CART(1-39); CART(42-89); Cocaine- and amphetamine-regulated |
| | | | transcript protein |
| 1437 | Q16595 | FRDA | Frataxin intermediate form; Frataxin mature form; Frataxin(56- |
| | | | 210); Frataxin(78-210); Frataxin, mitochondrial |
| 1438 | Q16625 | OCLN | Occludin |
| 1439 | Q16629 | SRSF7 | Serine/arginine-rich splicing factor 7 |
| 1440 | Q16630 | CPSF6 | Cleavage and polyadenylation specificity factor subunit 6 |
| 1441 | Q16643 | DREB | Drebrin |
| 1442 | Q16666 | IF16 | Gamma-interferon-inducible protein 16 |
| 1443 | Q16695 | H31T | Histone H3.1t |
| 1444 | Q16698 | DECR | 2,4-dienoyl-CoA reductase, mitochondrial |
| 1445 | Q16718 | NDUA5 | NADH dehydrogenase [ubiquinone] 1 alpha subcomplex subunit 5 |
| 1446 | Q16762 | THTR | Thiosulfate sulfurtransferase |
| 1447 | Q16775 | GLO2 | Hydroxyacylglutathione hydrolase, mitochondrial |
| 1448 | Q16777 | H2A2C | Histone H2A type 2-C |
| 1449 | Q16822 | PCKGM | Phosphoenolpyruvate carboxykinase [GTP], mitochondrial |
| 1450 | Q16836 | HCDH | Hydroxyacyl-coenzyme A dehydrogenase, mitochondrial |
| 1451 | Q16851 | UGPA | UTP--glucose-1-phosphate uridylyltransferase |
| 1452 | Q16890 | TPD53 | Tumor protein D53 |
| 1453 | Q16891 | MIC60 | MICOS complex subunit MIC60 |
| 1454 | Q1KMD3 | HNRL2 | Heterogeneous nuclear ribonucleoprotein U-like protein 2 |
| 1455 | Q1MSJ5 | CSPP1 | Centrosome and spindle pole-associated protein 1 |
| 1456 | Q29RF7 | PDS5A | Sister chromatid cohesion protein PDS5 homolog A |
| 1457 | Q2KHR3 | QSER1 | Glutamine and serine-rich protein 1 |
| 1458 | Q2M2I8 | AAK1 | AP2-associated protein kinase 1 |
| 1459 | Q2NL98 | VMAC | Vimentin-type intermediate filament-associated coiled-coil protein |
| 1460 | Q2TAL8 | QRIC1 | Glutamine-rich protein 1 |
| 1461 | Q32M84 | BTBDG | BTB/POZ domain-containing protein 16 |
| 1462 | Q32MZ4 | LRRF1 | Leucine-rich repeat flightless-interacting protein 1 |
| 1463 | Q3LXA3 | TKFC | ATP-dependent dihydroxyacetone kinase |
| 1464 | Q3SY69 | AL1L2 | Mitochondrial 10-formyltetrahydrofolate dehydrogenase |
| 1465 | Q3YEC7 | RABL6 | Rab-like protein 6 |
| 1466 | Q400G9 | AMZ1 | Archaemetzincin-1 |
| 1467 | Q49A26 | GLYR1 | Putative oxidoreductase GLYR1 |
| 1468 | Q49B96 | COX19 | Cytochrome c oxidase assembly protein COX19 |
| 1469 | Q4G0F5 | VP26B | Vacuolar protein sorting-associated protein 26B |
| 1470 | Q4KMP7 | TB10B | TBC1 domain family member 10B |
| 1471 | Q4KWH8 | PLCH1 | 1-phosphatidylinositol 4,5-bisphosphate phosphodiesterase eta-1 |
| 1472 | Q4L180 | FIL1L | Filamin A-interacting protein 1-like |
| 1473 | Q4V328 | GRAP1 | GRIP1-associated protein 1 |
| 1474 | Q4VC05 | BCL7A | B-cell CLL/lymphoma 7 protein family member A |
| 1475 | Q4VC31 | CCD58 | Coiled-coil domain-containing protein 58 |
| 1476 | Q53EL6 | PDCD4 | Programmed cell death protein 4 |
| 1477 | Q53F19 | NCBP3 | Uncharacterized protein C17orf85 |
| 1478 | Q53FZ2 | ACSM3 | Acyl-coenzyme A synthetase ACSM3, mitochondrial |
| 1479 | Q53GG5 | PDLI3 | PDZ and LIM domain protein 3 |
| 1480 | Q53GQ0 | DHB12 | Very-long-chain 3-oxoacyl-CoA reductase |
| 1481 | Q53S33 | BOLA3 | BolA-like protein 3 |
| 1482 | Q53SF7 | COBL1 | Cordon-bleu protein-like 1 |
| 1483 | Q53T59 | H1BP3 | HCLS1-binding protein 3 |
| 1484 | Q567U6 | CCD93 | Coiled-coil domain-containing protein 93 |
| 1485 | Q5BJF6 | ODFP2 | Outer dense fiber protein 2 |
| 1486 | Q5EBL8 | PDZ11 | PDZ domain-containing protein 11 |
| 1487 | Q5H9R7 | PP6R3 | Serine/threonine-protein phosphatase 6 regulatory subunit 3 |
| 1488 | Q5HYK7 | SH319 | SH3 domain-containing protein 19 |
| 1489 | Q5JR59 | MTUS2 | Microtubule-associated tumor suppressor candidate 2 |
| 1490 | Q5JRA6 | MIA3 | Melanoma inhibitory activity protein 3 |
| 1491 | Q5JSH3 | WDR44 | WD repeat-containing protein 44 |
| 1492 | Q5JSZ5 | PRC2B | Protein PRRC2B |
| 1493 | Q5JTD0 | TJAP1 | Tight junction-associated protein 1 |
| 1494 | Q5JTJ3 | COA6 | Cytochrome c oxidase assembly factor 6 homolog |
| 1495 | Q5JTV8 | TOIP1 | Torsin-1A-interacting protein 1 |
| 1496 | Q5JV73 | FRPD3 | FERM and PDZ domain-containing protein 3 |
| 1497 | Q5JXA9 | SIRB2 | Signal-regulatory protein beta-2 |
| 1498 | Q5M775 | CYTSB | Cytospin-B |
| 1499 | Q5NUL3 | FFAR4 | Free fatty acid receptor 4 |
| 1500 | Q5QJE6 | TDIF2 | Deoxynucleotidyltransferase terminal-interacting protein 2 |
| 1501 | Q5R3I4 | TTC38 | Tetratricopeptide repeat protein 38 |
| 1502 | Q5SSJ5 | HP1B3 | Heterochromatin protein 1-binding protein 3 |
| 1503 | Q5T013 | HYI | Putative hydroxypyruvate isomerase |
| 1504 | Q5T0F9 | C2D1B | Coiled-coil and C2 domain-containing protein 1B |
| 1505 | Q5T0N5 | FBP1L | Formin-binding protein 1-like |
| 1506 | Q5T0Z8 | CF132 | Uncharacterized protein C6orf132 |
| 1507 | Q5T5P2 | SKT | Sickle tail protein homolog |
| 1508 | Q5T6F2 | UBAP2 | Ubiquitin-associated protein 2 |
| 1509 | Q5T7N3 | KANK4 | KN motif and ankyrin repeat domain-containing protein 4 |
| 1510 | Q5T8P6 | RBM26 | RNA-binding protein 26 |
| 1511 | Q5T9A4 | ATD3B | ATPase family AAA domain-containing protein 3B |
| 1512 | Q5TCX8 | M3KL4 | Mitogen-activated protein kinase kinase kinase MLK4 |
| 1513 | Q5TZA2 | CROCC | Rootletin |
| 1514 | Q5U5X0 | LYRM7 | Complex III assembly factor LYRM7 |
| 1515 | Q5VSY0 | GKAP1 | G kinase-anchoring protein 1 |
| 1516 | Q5VUB5 | F1711 | Protein FAM171A1 |
| 1517 | Q5VZ89 | DEN4C | DENN domain-containing protein 4C |
| 1518 | Q5VZF2 | MBNL2 | Muscleblind-like protein 2 |
| 1519 | Q5VZK9 | CARL1 | Leucine-rich repeat-containing protein 16A |
| 1520 | Q5W0U4 | BSPRY | B box and SPRY domain-containing protein |
| 1521 | Q63HN8 | RN213 | E3 ubiquitin-protein ligase RNF213 |
| 1522 | Q63HR2 | TNS2 | Tensin-2 |
| 1523 | Q63ZY3 | KANK2 | KN motif and ankyrin repeat domain-containing protein 2 |
| 1524 | Q641Q2 | FA21A | WASH complex subunit FAM21A |
| 1525 | Q659C4 | LAR1B | La-related protein 1B |
| 1526 | Q66K74 | MAP1S | MAP1S heavy chain; MAP1S light chain; Microtubule-associated |
| | | | protein 1S |
| 1527 | Q66PJ3 | AR6P4 | ADP-ribosylation factor-like protein 6-interacting protein 4 |
| 1528 | Q68CZ2 | TENS3 | Tensin-3 |
| 1529 | Q6DD87 | ZN787 | Zinc finger protein 787 |
| 1530 | Q6EEV6 | SUMO4 | Small ubiquitin-related modifier 4 |
| 1531 | Q6FI81 | CPIN1 | Anamorsin |
| 1532 | Q6FIF0 | ZFAN6 | AN1-type zinc finger protein 6 |
| 1533 | Q6GMV3 | PTRD1 | Putative peptidyl-tRNA hydrolase PTRHD1 |
| 1534 | Q6GPI1 | CTRB2 | Chymotrypsin B2 chain A; Chymotrypsin B2 chain B; Chymotrypsin B2 |
| | | | chain C; Chymotrypsinogen B2 |
| 1535 | Q6IAA8 | LTOR1 | Ragulator complex protein LAMTOR1 |
| 1536 | Q6IEE8 | SN12L | Schlafen family member 12-like |
| 1537 | Q6IQ23 | PKHA7 | Pleckstrin homology domain-containing family A member 7 |
| 1538 | Q6JBY9 | CPZIP | CapZ-interacting protein |
| 1539 | Q6NUK1 | SCMC1 | Calcium-binding mitochondrial carrier protein SCaMC-1 |
| 1540 | Q6NVY1 | HIBCH | 3-hydroxyisobutyryl-CoA hydrolase, mitochondrial |
| 1541 | Q6NW29 | RWDD4 | RWD domain-containing protein 4 |
| 1542 | Q6NXT1 | ANR54 | Ankyrin repeat domain-containing protein 54 |
| 1543 | Q6NYC8 | PPR18 | Phostensin |
| 1544 | Q6NZI2 | PTRF | Polymerase I and transcript release factor |
| 1545 | Q6NZY4 | ZCHC8 | Zinc finger CCHC domain-containing protein 8 |
| 1546 | Q6P087 | RUSD3 | RNA pseudouridylate synthase domain-containing protein 3 |
| 1547 | Q6P1L8 | RM14 | 39S ribosomal protein L14, mitochondrial |
| 1548 | Q6P1N0 | C2D1A | Coiled-coil and C2 domain-containing protein 1A |
| 1549 | Q6P1N9 | TATD1 | Putative deoxyribonuclease TATDN1 |
| 1550 | Q6P1R3 | MSD2 | Myb/SANT-like DNA-binding domain-containing protein 2 |
| 1551 | Q6P2E9 | EDC4 | Enhancer of mRNA-decapping protein 4 |
| 1552 | Q6P4F2 | ADXL | Adrenodoxin-like protein, mitochondrial |
| 1553 | Q6P587 | FAHD1 | Acylpyruvase FAHD1, mitochondrial |
| 1554 | Q6P5R6 | RL22L | 60S ribosomal protein L22-like 1 |
| 1555 | Q6P6B1 | ERIC5 | Glutamate-rich protein 5 |
| 1556 | Q6PCB0 | VWA1 | von Willebrand factor A domain-containing protein 1 |
| 1557 | Q6PJT7 | ZC3HE | Zinc finger CCCH domain-containing protein 14 |
| 1558 | Q6PKG0 | LARP1 | La-related protein 1 |
| 1559 | Q6QNY0 | BL1S3 | Biogenesis of lysosome-related organelles complex 1 subunit 3 |
| 1560 | Q6QNY1 | BL1S2 | Biogenesis of lysosome-related organelles complex 1 subunit 2 |
| 1561 | Q6UN15 | FIP1 | Pre-mRNA 3′-end-processing factor FIP1 |
| 1562 | Q6UW15 | REG3G | Regenerating islet-derived protein 3-gamma; Regenerating islet-derived |
| | | | protein 3-gamma 15 kDa form |
| 1563 | Q6UW68 | TM205 | Transmembrane protein 205 |
| 1564 | Q6UWP8 | SBSN | Suprabasin |
| 1565 | Q6UX06 | OLFM4 | Olfactomedin-4 |
| 1566 | Q6UXB4 | CLC4G | C-type lectin domain family 4 member G |
| 1567 | Q6UXH1 | CREL2 | Cysteine-rich with EGF-like domain protein 2 |
| 1568 | Q6UXI9 | NPNT | Nephronectin |
| 1569 | Q6UXV4 | MIC27 | MICOS complex subunit MIC27 |
| 1570 | Q6WCQ1 | MPRIP | Myosin phosphatase Rho-interacting protein |
| 1571 | Q6Y7W6 | PERQ2 | PERQ amino acid-rich with GYF domain-containing protein 2 |
| 1572 | Q6YN16 | HSDL2 | Hydroxysteroid dehydrogenase-like protein 2 |
| 1573 | Q6YP21 | KAT3 | Kynurenine-oxoglutarate transaminase 3 |
| 1574 | Q6ZRV2 | FA83H | Protein FAM83H |
| 1575 | Q6ZSR9 | YJ005 | Uncharacterized protein FLJ45252 |
| 1576 | Q6ZUJ8 | BCAP | Phosphoinositide 3-kinase adapter protein 1 |
| 1577 | Q6ZUS6 | CC149 | Coiled-coil domain-containing protein 149 |
| 1578 | Q6ZVF9 | GRIN3 | G protein-regulated inducer of neurite outgrowth 3 |
| 1579 | Q6ZVM7 | TM1L2 | TOM1-like protein 2 |
| 1580 | Q70E73 | RAPH1 | Ras-associated and pleckstrin homology domains-containing protein 1 |
| 1581 | Q71RC2 | LARP4 | La-related protein 4 |
| 1582 | Q71UM5 | RS27L | 40S ribosomal protein S27-like |
| 1583 | Q7KZF4 | SND1 | Staphylococcal nuclease domain-containing protein 1 |
| 1584 | Q7L266 | ASGL1 | Isoaspartyl peptidase/L-asparaginase; Isoaspartyl peptidase/L- |
| | | | asparaginase alpha chain |
| 1585 | Q7L2H7 | EIF3M | Eukaryotic translation initiation factor 3 subunit M |
| 1586 | Q7L2J0 | MEPCE | 7SK snRNA methylphosphate capping enzyme |
| 1587 | Q7L311 | ARMX2 | Armadillo repeat-containing X-linked protein 2 |
| 1588 | Q7L4I2 | RSRC2 | Arginine/serine-rich coiled-coil protein 2 |
| 1589 | Q7LBR1 | CHM1B | Charged multivesicular body protein 1b |
| 1590 | Q7RTS1 | BHA15 | Class A basic helix-loop-helix protein 15 |
| 1591 | Q7RTV0 | PHF5A | PHD finger-like domain-containing protein 5A |
| 1592 | Q7Z2W4 | ZCCHV | Zinc finger CCCH-type antiviral protein 1 |
| 1593 | Q7Z3E2 | CC186 | Coiled-coil domain-containing protein 186 |
| 1594 | Q7Z3K3 | POGZ | Pogo transposable element with ZNF domain |
| 1595 | Q7Z3T8 | ZFY16 | Zinc finger FYVE domain-containing protein 16 |
| 1596 | Q7Z406 | MYH14 | Myosin-14 |
| 1597 | Q7Z417 | NUFP2 | Nuclear fragile X mental retardation-interacting protein 2 |
| 1598 | Q7Z422 | SZRD1 | SUZ domain-containing protein 1 |
| 1599 | Q7Z434 | MAVS | Mitochondrial antiviral-signaling protein |
| 1600 | Q7Z4H3 | HDDC2 | HD domain-containing protein 2 |
| 1601 | Q7Z4V5 | HDGR2 | Hepatoma-derived growth factor-related protein 2 |
| 1602 | Q7Z4W1 | DCXR | L-xylulose reductase |
| 1603 | Q7Z5L9 | I2BP2 | Interferon regulatory factor 2-binding protein 2 |
| 1604 | Q7Z6B0 | CCD91 | Coiled-coil domain-containing protein 91 |
| 1605 | Q7Z6I8 | CE024 | UPF0461 protein C5orf24 |
| 1606 | Q7Z6K5 | ARPIN | Arpin |
| 1607 | Q7Z6Z7 | HUWE1 | E3 ubiquitin-protein ligase HUWE1 |
| 1608 | Q7Z739 | YTHD3 | YTH domain-containing family protein 3 |
| 1609 | Q7Z7G0 | TARSH | Target of Nesh-SH3 |
| 1610 | Q7Z7K0 | COXM1 | COX assembly mitochondrial protein homolog |
| 1611 | Q7Z7K6 | CENPV | Centromere protein V |
| 1612 | Q7Z7L8 | CK096 | Uncharacterized protein C11orf96 |
| 1613 | Q86SX6 | GLRX5 | Glutaredoxin-related protein 5, mitochondrial |
| 1614 | Q86U28 | ISCA2 | Iron-sulfur cluster assembly 2 homolog, mitochondrial |
| 1615 | Q86U42 | PABP2 | Polyadenylate-binding protein 2 |
| 1616 | Q86U44 | MTA70 | N6-adenosine-methyltransferase 70 kDa subunit |
| 1617 | Q86UE4 | LYRIC | Protein LYRIC |
| 1618 | Q86UP2 | KTN1 | Kinectin |
| 1619 | Q86UP6 | CUZD1 | CUB and zona pellucida-like domain-containing protein 1 |
| 1620 | Q86UU1 | PHLB1 | Pleckstrin homology-like domain family B member 1 |
| 1621 | Q86V48 | LUZP1 | Leucine zipper protein 1 |
| 1622 | Q86V81 | THOC4 | THO complex subunit 4 |
| 1623 | Q86VB7 | C163A | Scavenger receptor cysteine-rich type 1 protein M130; Soluble CD163 |
| 1624 | Q86VM9 | ZCH18 | Zinc finger CCCH domain-containing protein 18 |
| 1625 | Q86WW8 | COA5 | Cytochrome c oxidase assembly factor 5 |
| 1626 | Q86WZ6 | ZN227 | Zinc finger protein 227 |
| 1627 | Q86X29 | LSR | Lipolysis-stimulated lipoprotein receptor |
| 1628 | Q86X76 | NIT1 | Nitrilase homolog 1 |
| 1629 | Q86XE5 | HOGA1 | 4-hydroxy-2-oxoglutarate aldolase, mitochondrial |
| 1630 | Q86XP3 | DDX42 | ATP-dependent RNA helicase DDX42 |
| 1631 | Q86XX4 | FRAS1 | Extracellular matrix protein FRAS1 |
| 1632 | Q86Y82 | STX12 | Syntaxin-12 |
| 1633 | Q86YB8 | ERO1B | ERO1-like protein beta |
| 1634 | Q86YL5 | TDRP | Testis development-related protein |
| 1635 | Q86YP4 | P66A | Transcriptional repressor p66-alpha |
| 1636 | Q86YR5 | GPSM1 | G-protein-signaling modulator 1 |
| 1637 | Q86YW9 | MD12L | Mediator of RNA polymerase II transcription subunit 12-like protein |
| 1638 | Q8IU81 | I2BP1 | Interferon regulatory factor 2-binding protein 1 |
| 1639 | Q8IUD2 | RB6I2 | ELKS/Rab6-interacting/CAST family member 1 |
| 1640 | Q8IUE6 | H2A2B | Histone H2A type 2-B |
| 1641 | Q8IVF2 | AHNK2 | Protein AHNAK2 |
| 1642 | Q8IVF4 | DYH10 | Dynein heavy chain 10, axonemal |
| 1643 | Q8IVM0 | CCD50 | Coiled-coil domain-containing protein 50 |
| 1644 | Q8IVN3 | MSTN1 | Musculoskeletal embryonic nuclear protein 1 |
| 1645 | Q8IVN8 | SBSPO | Somatomedin-B and thrombospondin type-1 domain-containing protein |
| 1646 | Q8IWE2 | NXP20 | Protein NOXP20 |
| 1647 | Q8IWW6 | RHG12 | Rho GTPase-activating protein 12 |
| 1648 | Q8IWX8 | CHERP | Calcium homeostasis endoplasmic reticulum protein |
| 1649 | Q8IX12 | CCAR1 | Cell division cycle and apoptosis regulator protein 1 |
| 1650 | Q8IXM2 | BAP18 | Chromatin complexes subunit BAP18 |
| 1651 | Q8IXQ4 | GPAM1 | GPALPP motifs-containing protein 1 |
| 1652 | Q8IY67 | RAVR1 | Ribonucleoprotein PTB-binding 1 |
| 1653 | Q8IYB3 | SRRM1 | Serine/arginine repetitive matrix protein 1 |
| 1654 | Q8IYB8 | SUV3 | ATP-dependent RNA helicase SUPV3L1, mitochondrial |
| 1655 | Q8IYJ3 | SYTL1 | Synaptotagmin-like protein 1 |
| 1656 | Q8IYL3 | CA174 | UPF0688 protein C1orf174 |
| 1657 | Q8IYQ7 | THNS1 | Threonine synthase-like 1 |
| 1658 | Q8IZ21 | PHAR4 | Phosphatase and actin regulator 4 |
| 1659 | Q8IZA0 | K319L | Dyslexia-associated protein KIAA0319-like protein |
| 1660 | Q8IZP0 | ABH | Abl interactor 1 |
| 1661 | Q8IZQ5 | SELH | Selenoprotein H |
| 1662 | Q8N0T1 | CH059 | Uncharacterized protein C8orf59 |
| 1663 | Q8N163 | CCAR2 | Cell cycle and apoptosis regulator protein 2 |
| 1664 | Q8N183 | MIMIT | Mimitin, mitochondrial |
| 1665 | Q8N1A0 | KT222 | Keratin-like protein KRT222 |
| 1666 | Q8N1G4 | LRC47 | Leucine-rich repeat-containing protein 47 |
| 1667 | Q8N2S1 | LTBP4 | Latent-transforming growth factor beta-binding protein 4 |
| 1668 | Q8N3J5 | PPM1K | Protein phosphatase 1K, mitochondrial |
| 1669 | Q8N3V7 | SYNPO | Synaptopodin |
| 1670 | Q8N4P3 | MESH1 | Guanosine-3′,5′-bis(diphosphate) 3′-pyrophosphohydrolase MESH1 |
| 1671 | Q8N4Q1 | MIA40 | Mitochondrial intermembrane space import and assembly protein 40 |
| 1672 | Q8N4T8 | CBR4 | Carbonyl reductase family member 4 |
| 1673 | Q8N5J2 | FA63A | Protein FAM63A |
| 1674 | Q8N5K1 | CISD2 | CDGSH iron-sulfur domain-containing protein 2 |
| 1675 | Q8N5N7 | RM50 | 39S ribosomal protein L50, mitochondrial |
| 1676 | Q8N684 | CPSF7 | Cleavage and polyadenylation specificity factor subunit 7 |
| 1677 | Q8N6D5 | ANR29 | Ankyrin repeat domain-containing protein 29 |
| 1678 | Q8N6H7 | ARFG2 | ADP-ribosylation factor GTPase-activating protein 2 |
| 1679 | Q8N6T3 | ARFG1 | ADP-ribosylation factor GTPase-activating protein 1 |
| 1680 | Q8N8S7 | ENAH | Protein enabled homolog |
| 1681 | Q8N9L9 | ACOT4 | Acyl-coenzyme A thioesterase 4 |
| 1682 | Q8N9N8 | EIF1A | Probable RNA-binding protein EIF1AD |
| 1683 | Q8NAA4 | A16L2 | Autophagy-related protein 16-2 |
| 1684 | Q8NBJ4 | GOLM1 | Golgi membrane protein 1 |
| 1685 | Q8NBJ7 | SUMF2 | Sulfatase-modifying factor 2 |
| 1686 | Q8NBJ9 | SIDT2 | SID1 transmembrane family member 2 |
| 1687 | Q8NBS9 | TXND5 | Thioredoxin domain-containing protein 5 |
| 1688 | Q8NBX0 | SCPDL | Saccharopine dehydrogenase-like oxidoreductase |
| 1689 | Q8NC51 | PAIRB | Plasminogen activator inhibitor 1 RNA-binding protein |
| 1690 | Q8NC56 | LEMD2 | LEM domain-containing protein 2 |
| 1691 | Q8NC96 | NECP1 | Adaptin ear-binding coat-associated protein 1 |
| 1692 | Q8NCA5 | FA98A | Protein FAM98A |
| 1693 | Q8NCW5 | NNRE | NAD(P)H-hydrate epimerase |
| 1694 | Q8ND24 | RN214 | RING finger protein 214 |
| 1695 | Q8ND56 | LS14A | Protein LSM14 homolog A |
| 1696 | Q8NDC0 | MISSL | MAPK-interacting and spindle-stabilizing protein-like |
| 1697 | Q8NE62 | CHDH | Choline dehydrogenase, mitochondrial |
| 1698 | Q8NE71 | ABCF1 | ATP-binding cassette sub-family F member 1 |
| 1699 | Q8NEP7 | KLDC9 | Kelch domain-containing protein 9 |
| 1700 | Q8NFC6 | BD1L1 | Biorientation of chromosomes in cell division protein 1-like 1 |
| 1701 | Q8NFD5 | ARI1B | AT-rich interactive domain-containing protein 1B |
| 1702 | Q8NFH5 | NUP53 | Nucleoporin NUP53 |
| 1703 | Q8NFQ8 | TOIP2 | Torsin-1A-interacting protein 2 |
| 1704 | Q8NFU3 | TSTD1 | Thiosulfate sulfurtransferase/rhodanese-like domain-containing protein 1 |
| 1705 | Q8NFW8 | NEUA | N-acylneuraminate cytidylyltransferase |
| 1706 | Q8NHM4 | TRY6 | Putative trypsin-6 |
| 1707 | Q8NHZ8 | CDC26 | Anaphase-promoting complex subunit CDC26 |
| 1708 | Q8NI22 | MCFD2 | Multiple coagulation factor deficiency protein 2 |
| 1709 | Q8NI60 | ADCK3 | Atypical kinase ADCK3, mitochondrial |
| 1710 | Q8TAE8 | G45IP | Growth arrest and DNA damage-inducible proteins-interacting protein 1 |
| 1711 | Q8TAQ2 | SMRC2 | SWI/SNF complex subunit SMARCC2 |
| 1712 | Q8TAT6 | NPL4 | Nuclear protein localization protein 4 homolog |
| 1713 | Q8TB37 | NUBPL | Iron-sulfur protein NUBPL |
| 1714 | Q8TBA6 | GOGA5 | Golgin subfamily A member 5 |
| 1715 | Q8TC07 | TBC15 | TBC1 domain family member 15 |
| 1716 | Q8TCC3 | RM30 | 39S ribosomal protein L30, mitochondrial |
| 1717 | Q8TCS8 | PNPT1 | Polyribonucleotide nucleotidyltransferase 1, mitochondrial |
| 1718 | Q8TEA8 | DTD1 | D-tyrosyl-tRNA(Tyr) deacylase 1 |
| 1719 | Q8TF45 | ZN418 | Zinc finger protein 418 |
| 1720 | Q8TF65 | GIPC2 | PDZ domain-containing protein GIPC2 |
| 1721 | Q8TF71 | MOT10 | Monocarboxylate transporter 10 |
| 1722 | Q8TF74 | WIPF2 | WAS/WASL-interacting protein family member 2 |
| 1723 | Q8WU79 | SMAP2 | Stromal membrane-associated protein 2 |
| 1724 | Q8WU90 | ZC3HF | Zinc finger CCCH domain-containing protein 15 |
| 1725 | Q8WUD4 | CCD12 | Coiled-coil domain-containing protein 12 |
| 1726 | Q8WUH6 | TM263 | Transmembrane protein 263 |
| 1727 | Q8WUM4 | PDC6I | Programmed cell death 6-interacting protein |
| 1728 | Q8WUP2 | FBLI1 | Filamin-binding LIM protein 1 |
| 1729 | Q8VW99 | ZFN2B | AN1-type zinc finger protein 2B |
| 1730 | Q8VWJ2 | NUDC2 | NudC domain-containing protein 2 |
| 1731 | Q8VWK2 | SNR27 | U4/U6.U5 small nuclear ribonucleoprotein 27 kDa protein |
| 1732 | Q8VWM8 | SCFD1 | Sec1 family domain-containing protein 1 |
| 1733 | Q8WW4 | POF1B | Protein POF1B |
| 1734 | Q8WW12 | PCNP | PEST proteolytic signal-containing nuclear protein |
| 1735 | Q8WWI1 | LMO7 | LIM domain only protein 7 |
| 1736 | Q8WWM7 | ATX2L | Ataxin-2-like protein |
| 1737 | Q8WWX9 | SELM | Selenoprotein M |
| 1738 | Q8WX93 | PALLD | Palladin |
| 1739 | Q8WXC6 | MYOV2 | Myeloma-overexpressed gene 2 protein |
| 1740 | Q8WXD2 | SCG3 | Secretogranin-3 |
| 1741 | Q8WXD9 | CSKI1 | Caskin-1 |
| 1742 | Q8WXF1 | PSPC1 | Paraspeckle component 1 |
| 1743 | Q8WXI7 | MUC16 | Mucin-16 |
| 1744 | Q8WXX5 | DNJC9 | DnaJ homolog subfamily C member 9 |
| 1745 | Q8WY36 | BBX | HMG box transcription factor BBX |
| 1746 | Q8WZ42 | TITIN | Titin |
| 1747 | Q8WZA0 | LZIC | Protein LZIC |
| 1748 | Q8WZA9 | IRGQ | Immunity-related GTPase family Q protein |
| 1749 | Q92499 | DDX1 | ATP-dependent RNA helicase DDX1 |
| 1750 | Q92506 | DHB8 | Estradiol 17-beta-dehydrogenase 8 |
| 1751 | Q92508 | PIEZ1 | Piezo-type mechanosensitive ion channel component 1 |
| 1752 | Q92520 | FAM3C | Protein FAM3C |
| 1753 | Q92522 | H1X | Histone H1x |
| 1754 | Q92541 | RTF1 | RNA polymerase-associated protein RTF1 homolog |
| 1755 | Q92575 | UBXN4 | UBX domain-containing protein 4 |
| 1756 | Q92576 | PHF3 | PHD finger protein 3 |
| 1757 | Q92597 | NDRG1 | Protein NDRG1 |
| 1758 | Q92598 | HS105 | Heat shock protein 105 kDa |
| 1759 | Q92599 | 39692 | Septin-8 |
| 1760 | Q92609 | TBCD5 | TBC1 domain family member 5 |
| 1761 | Q92614 | MY18A | Unconventional myosin-XVIIIa |
| 1762 | Q92615 | LAR4B | La-related protein 4B |
| 1763 | Q92620 | PRP16 | Pre-mRNA-splicing factor ATP-dependent RNA helicase PRP16 |
| 1764 | Q92665 | RT31 | 28S ribosomal protein S31, mitochondrial |
| 1765 | Q92667 | AKAP1 | A-kinase anchor protein 1, mitochondrial |
| 1766 | Q92688 | AN32B | Acidic leucine-rich nuclear phosphoprotein 32 family member B |
| 1767 | Q92733 | PRCC | Proline-rich protein PRCC |
| 1768 | Q92734 | TFG | Protein TFG |
| 1769 | Q92747 | ARC1A | Actin-related protein 2/3 complex subunit 1A |
| 1770 | Q92777 | SYN2 | Synapsin-2 |
| 1771 | Q92783 | STAM1 | Signal transducing adapter molecule 1 |
| 1772 | Q92785 | REQU | Zinc finger protein ubi-d4 |
| 1773 | Q92796 | DLG3 | Disks large homolog 3 |
| 1774 | Q92797 | SYMPK | Symplekin |
| 1775 | Q92804 | RBP56 | TATA-binding protein-associated factor 2N |
| 1776 | Q92805 | GOGA1 | Golgin subfamily A member 1 |
| 1777 | Q92823 | NRCAM | Neuronal cell adhesion molecule |
| 1778 | Q92841 | DDX17 | Probable ATP-dependent RNA helicase DDX17 |
| 1779 | Q92878 | RAD50 | DNA repair protein RAD50 |
| 1780 | Q92896 | GSLG1 | Golgi apparatus protein 1 |
| 1781 | Q92900 | RENT1 | Regulator of nonsense transcripts 1 |
| 1782 | Q92905 | CSN5 | COP9 signalosome complex subunit 5 |
| 1783 | Q92917 | GPKOW | G patch domain and KOW motifs-containing protein |
| 1784 | Q92922 | SMRC1 | SWI/SNF complex subunit SMARCC1 |
| 1785 | Q92934 | BAD | Bcl2-associated agonist of cell death |
| 1786 | Q92945 | FUBP2 | Far upstream element-binding protein 2 |
| 1787 | Q92973 | TNPO1 | Transportin-1 |
| 1788 | Q93009 | UBP7 | Ubiquitin carboxyl-terminal hydrolase 7 |
| 1789 | Q93052 | LPP | Lipoma-preferred partner |
| 1790 | Q93062 | RBPMS | RNA-binding protein with multiple splicing |
| 1791 | Q969E4 | TCAL3 | Transcription elongation factor A protein-like 3 |
| 1792 | Q969G3 | SMCE1 | SWI/SNF-related matrix-associated actin-dependent |
| | | | regulator of chromatin subfamily E member 1 |
| 1793 | Q969G5 | PRDBP | Protein kinase C delta-binding protein |
| 1794 | Q969H8 | MYDGF | Myeloid-derived growth factor |
| 1795 | Q969Q0 | RL36L | 60S ribosomal protein L36a-like |
| 1796 | Q969T9 | WBP2 | WW domain-binding protein 2 |
| 1797 | Q96A00 | PP14A | Protein phosphatase 1 regulatory subunit 14A |
| 1798 | Q96A26 | F162A | Protein FAM162A |
| 1799 | Q96A33 | CCD47 | Coiled-coil domain-containing protein 47 |
| 1800 | Q96A49 | SYAP1 | Synapse-associated protein 1 |
| 1801 | Q96A83 | COQA1 | Collagen alpha-1(XXVI) chain |
| 1802 | Q96AB3 | ISOC2 | Isochorismatase domain-containing protein 2, mitochondrial |
| 1803 | Q96AE4 | FUBP1 | Far upstream element-binding protein 1 |
| 1804 | Q96AG3 | S2546 | Solute carrier family 25 member 46 |
| 1805 | Q96AG4 | LRC59 | Leucine-rich repeat-containing protein 59 |
| 1806 | Q96AT1 | K1143 | Uncharacterized protein KIAA1143 |
| 1807 | Q96B36 | AKTS1 | Proline-rich AKT1 substrate 1 |
| 1808 | Q96B54 | ZN428 | Zinc finger protein 428 |
| 1809 | Q96B97 | SH3K1 | SH3 domain-containing kinase-binding protein 1 |
| 1810 | Q96BJ3 | AIDA | Axin interactor, dorsalization-associated protein |
| 1811 | Q96BP3 | PPWD1 | Peptidylprolyl isomerase domain and WD repeat-containing protein 1 |
| 1812 | Q96C01 | F136A | Protein FAM136A |
| 1813 | Q96C19 | EFHD2 | EF-hand domain-containing protein D2 |
| 1814 | Q96C23 | GALM | Aldose 1-epimerase |
| 1815 | Q96C36 | P5CR2 | Pyrroline-5-carboxylate reductase 2 |
| 1816 | Q96C86 | DCPS | m7GpppX diphosphatase |
| 1817 | Q96C90 | PP14B | Protein phosphatase 1 regulatory subunit 14B |
| 1818 | Q96CT7 | CC124 | Coiled-coil domain-containing protein 124 |
| 1819 | Q96CV9 | OPTN | Optineurin |
| 1820 | Q96CX2 | KCD12 | BTB/POZ domain-containing protein KCTD12 |
| 1821 | Q96D15 | RCN3 | Reticulocalbin-3 |
| 1822 | Q96DC8 | ECHD3 | Enoyl-CoA hydratase domain-containing protein 3, mitochondrial |
| 1823 | Q96DG6 | CMBL | Carboxymethylenebutenolidase homolog |
| 1824 | Q96DN0 | ERP27 | Endoplasmic reticulum resident protein 27 |
| 1825 | Q96DZ1 | ERLEC | Endoplasmic reticulum lectin 1 |
| 1826 | Q96E11 | RRFM | Ribosome-recycling factor, mitochondrial |
| 1827 | Q96E29 | MTEF3 | Transcription termination factor 3, mitochondrial |
| 1828 | Q96E39 | RMXL1 | RNA binding motif protein, X-linked-like-1 |
| 1829 | Q96EE3 | SEH1 | Nucleoporin SEH1 |
| 1830 | Q96EI5 | TCAL4 | Transcription elongation factor A protein-like 4 |
| 1831 | Q96EL3 | RM53 | 39S ribosomal protein L53, mitochondrial |
| 1832 | Q96EP5 | DAZP1 | DAZ-associated protein 1 |
| 1833 | Q96ER9 | CCD51 | Coiled-coil domain-containing protein 51 |
| 1834 | Q96EV2 | RBM33 | RNA-binding protein 33 |
| 1835 | Q96EY8 | MMAB | Cob(I)yrinic acid a,c-diamide adenosyltransferase, mitochondrial |
| 1836 | Q96FF7 | YS003 | Uncharacterized protein LOC113230 |
| 1837 | Q96FW1 | OTUB1 | Ubiquitin thioesterase OTUB1 |
| 1838 | Q96G03 | PGM2 | Phosphoglucomutase-2 |
| 1839 | Q96GK7 | FAH2A | Fumarylacetoacetate hydrolase domain-containing protein 2A |
| 1840 | Q96GX9 | MTNB | Methylthioribulose-1-phosphate dehydratase |
| 1841 | Q96GY0 | ZC21A | Zinc finger C2HC domain-containing protein 1A |
| 1842 | Q96HC4 | PDLI5 | PDZ and LIM domain protein 5 |
| 1843 | Q96HE7 | ERO1A | ERO1-like protein alpha |
| 1844 | Q96HR9 | REEP6 | Receptor expression-enhancing protein 6 |
| 1845 | Q96HY6 | DDRGK | DDRGK domain-containing protein 1 |
| 1846 | Q96I15 | SCLY | Selenocysteine lyase |
| 1847 | Q96I24 | FUBP3 | Far upstream element-binding protein 3 |
| 1848 | Q96I25 | SPF45 | Splicing factor 45 |
| 1849 | Q96I99 | SUCB2 | Succinyl-CoA ligase [GDP-forming] subunit beta, mitochondrial |
| 1850 | Q96IJ6 | GMPPA | Mannose-1-phosphate guanyltransferase alpha |
| 1851 | Q96IU4 | ABHEB | Alpha/beta hydrolase domain-containing protein 14B |
| 1852 | Q96IZ0 | PAWR | PRKC apoptosis WT1 regulator protein |
| 1853 | Q96IZ7 | RSRC1 | Serine/Arginine-related protein 53 |
| 1854 | Q96J84 | KIRR1 | Kin of IRRE-like protein 1 |
| 1855 | Q96JA1 | LRIG1 | Leucine-rich repeats and immunoglobulin-like domains protein 1 |
| 1856 | Q96JB5 | CK5P3 | CDK5 regulatory subunit-associated protein 3 |
| 1857 | Q96JH7 | VCIP1 | Deubiquitinating protein VCIP135 |
| 1858 | Q96JP5 | ZFP91 | E3 ubiquitin-protein ligase ZFP91 |
| 1859 | Q96JQ2 | CLMN | Calmin |
| 1860 | Q96JY6 | PDLI2 | PDZ and LIM domain protein 2 |
| 1861 | Q96K17 | BT3L4 | Transcription factor BTF3 homolog 4 |
| 1862 | Q96KC8 | DNJC1 | DnaJ homolog subfamily C member 1 |
| 1863 | Q96KG9 | NTKL | N-terminal kinase-like protein |
| 1864 | Q96KP4 | CNDP2 | Cytosolic non-specific dipeptidase |
| 1865 | Q96KR1 | ZFR | Zinc finger RNA-binding protein |
| 1866 | Q96M27 | PRRC1 | Protein PRRC1 |
| 1867 | Q96MW1 | CCD43 | Coiled-coil domain-containing protein 43 |
| 1868 | Q96NB3 | ZN830 | Zinc finger protein 830 |
| 1869 | Q96P44 | COLA1 | Collagen alpha-1(XXI) chain |
| 1870 | Q96PE7 | MCEE | Methylmalonyl-CoA epimerase, mitochondrial |
| 1871 | Q96PK6 | RBM14 | RNA-binding protein 14 |
| 1872 | Q96PU5 | NED4L | E3 ubiquitin-protein ligase NEDD4-like |
| 1873 | Q96Q06 | PLIN4 | Perilipin-4 |
| 1874 | Q96Q89 | KI20B | Kinesin-like protein KIF20B |
| 1875 | Q96QK1 | VPS35 | Vacuolar protein sorting-associated protein 35 |
| 1876 | Q96QR8 | PURB | Transcriptional activator protein Pur-beta |
| 1877 | Q96QS6 | KPSH2 | Serine/threonine-protein kinase H2 |
| 1878 | Q96QT6 | PHF12 | PHD finger protein 12 |
| 1879 | Q96QZ7 | MAGI1 | Membrane-associated guanylate kinase, WW and PDZ domain- |
| | | | containing protein 1 |
| 1880 | Q96RF0 | SNX18 | Sorting nexin-18 |
| 1881 | Q96RL1 | UIMC1 | BRCA1-A complex subunit RAP80 |
| 1882 | Q96RP9 | EFGM | Elongation factor G, mitochondrial |
| 1883 | Q96RQ3 | MCCA | Methylcrotonoyl-CoA carboxylase subunit alpha, mitochondrial |
| 1884 | Q96S55 | WRIP1 | ATPase WRNIP1 |
| 1885 | Q96S66 | CLCC1 | Chloride channel CLIC-like protein 1 |
| 1886 | Q96SB3 | NEB2 | Neurabin-2 |
| 1887 | Q96ST2 | IWS1 | Protein IWS1 homolog |
| 1888 | Q96ST3 | SIN3A | Paired amphipathic helix protein Sin3a |
| 1889 | Q96T37 | RBM15 | Putative RNA-binding protein 15 |
| 1890 | Q96T51 | RUFY1 | RUN and FYVE domain-containing protein 1 |
| 1891 | Q96T58 | MINT | Msx2-interacting protein |
| 1892 | Q96TA1 | NIBL1 | Niban-like protein 1 |
| 1893 | Q99417 | MYCBP | C-Myc-binding protein |
| 1894 | Q99426 | TBCB | Tubulin-folding cofactor B |
| 1895 | Q99436 | PSB7 | Proteasome subunit beta type-7 |
| 1896 | Q99439 | CNN2 | Calponin-2 |
| 1897 | Q99459 | CDC5L | Cell division cycle 5-like protein |
| 1898 | Q99460 | PSMD1 | 26S proteasome non-ATPase regulatory subunit 1 |
| 1899 | Q99471 | PFD5 | Prefoldin subunit 5 |
| 1900 | Q99497 | PARK7 | Protein deglycase DJ-1 |
| 1901 | Q99536 | VAT1 | Synaptic vesicle membrane protein VAT-1 homolog |
| 1902 | Q99538 | LGMN | Legumain |
| 1903 | Q99569 | PKP4 | Plakophilin-4 |
| 1904 | Q99584 | S10AD | Protein S100-A13 |
| 1905 | Q99590 | SCAFB | Protein SCAF11 |
| 1906 | Q99614 | TTC1 | Tetratricopeptide repeat protein 1 |
| 1907 | Q99622 | C10 | Protein C10 |
| 1908 | Q99623 | PHB2 | Prohibitin-2 |
| 1909 | Q99700 | ATX2 | Ataxin-2 |
| 1910 | Q99714 | HCD2 | 3-hydroxyacyl-CoA dehydrogenase type-2 |
| 1911 | Q99715 | COCA1 | Collagen alpha-1(XII) chain |
| 1912 | Q99729 | ROAA | Heterogeneous nuclear ribonucleoprotein A/B |
| 1913 | Q99733 | NP1L4 | Nucleosome assembly protein 1-like 4 |
| 1914 | Q99747 | SNAG | Gamma-soluble NSF attachment protein |
| 1915 | Q99757 | THIOM | Thioredoxin, mitochondrial |
| 1916 | Q99798 | ACON | Aconitate hydratase, mitochondrial |
| 1917 | Q99805 | TM9S2 | Transmembrane 9 superfamily member 2 |
| 1918 | Q99829 | CPNE1 | Copine-1 |
| 1919 | Q99832 | TCPH | T-complex protein 1 subunit eta |
| 1920 | Q99895 | CTRC | Chymotrypsin-C |
| 1921 | Q99933 | BAG1 | BAG family molecular chaperone regulator 1 |
| 1922 | Q99959 | PKP2 | Plakophilin-2 |
| 1923 | Q99961 | SH3G1 | Endophilin-A2 |
| 1924 | Q99969 | RARR2 | Retinoic acid receptor responder protein 2 |
| 1925 | Q99996 | AKAP9 | A-kinase anchor protein 9 |
| 1926 | Q9BPX5 | ARP5L | Actin-related protein 2/3 complex subunit 5-like protein |
| 1927 | Q9BPY8 | HOP | Homeodomain-only protein |
| 1928 | Q9BQ69 | MACD1 | O-acetyl-ADP-ribose deacetylase MACROD1 |
| 1929 | Q9BQA1 | MEP50 | Methylosome protein 50 |
| 1930 | Q9BQE3 | TBA1C | Tubulin alpha-1C chain |
| 1931 | Q9BQI0 | AIF1L | Allograft inflammatory factor 1-like |
| 1932 | Q9BQS8 | FYCO1 | FYVE and coiled-coil domain-containing protein 1 |
| 1933 | Q9BR76 | COR1B | Coronin-1B |
| 1934 | Q9BRA0 | LSMD1 | N-alpha-acetyltransferase 38, NatC auxiliary subunit |
| 1935 | Q9BRA2 | TXD17 | Thioredoxin domain-containing protein 17 |
| 1936 | Q9BRJ6 | CG050 | Uncharacterized protein C7orf50 |
| 1937 | Q9BRK5 | CAB45 | 45 kDa calcium-binding protein |
| 1938 | Q9BRP8 | PYM1 | Partner of Y14 and mago |
| 1939 | Q9BRQ6 | MIC25 | MICOS complex subunit MIC25 |
| 1940 | Q9BRT3 | MIEN1 | Migration and invasion enhancer 1 |
| 1941 | Q9BRX2 | PELO | Protein pelota homolog |
| 1942 | Q9BS26 | ERP44 | Endoplasmic reticulum resident protein 44 |
| 1943 | Q9BS40 | LXN | Latexin |
| 1944 | Q9BSH4 | TACO1 | Translational activator of cytochrome c oxidase 1 |
| 1945 | Q9BT09 | CNPY3 | Protein canopy homolog 3 |
| 1946 | Q9BTC0 | DIDO1 | Death-inducer obliterator 1 |
| 1947 | Q9BTD8 | RBM42 | RNA-binding protein 42 |
| 1948 | Q9BTE1 | DCTN5 | Dynactin subunit 5 |
| 1949 | Q9BTT0 | AN32E | Acidic leucine-rich nuclear phosphoprotein 32 family member E |
| 1950 | Q9BTZ2 | DHRS4 | Dehydrogenase/reductase SDR family member 4 |
| 1951 | Q9BU68 | PR15L | Proline-rich protein 15-like protein |
| 1952 | Q9BUH6 | PAXX | Protein PAXX |
| 1953 | Q9BUJ2 | HNRL1 | Heterogeneous nuclear ribonucleoprotein U-like protein 1 |
| 1954 | Q9BUQ8 | DDX23 | Probable ATP-dependent RNA helicase DDX23 |
| 1955 | Q9BUT1 | BDH2 | 3-hydroxybutyrate dehydrogenase type 2 |
| 1956 | Q9BV20 | MTNA | Methylthioribose-1-phosphate isomerase |
| 1957 | Q9BV40 | VAMP8 | Vesicle-associated membrane protein 8 |
| 1958 | Q9BVC5 | ASHWN | Ashwin |
| 1959 | Q9BVG4 | PBDC1 | Protein PBDC1 |
| 1960 | Q9BVJ6 | UT14A | U3 small nucleolar RNA-associated protein 14 homolog A |
| 1961 | Q9BVK6 | TMED9 | Transmembrane emp24 domain-containing protein 9 |
| 1962 | Q9BVL2 | NUP58 | Nucleoporin p58/p45 |
| 1963 | Q9BVM2 | DPCD | Protein DPCD |
| 1964 | Q9BVM4 | GGACT | Gamma-glutamylaminecyclotransferase |
| 1965 | Q9BW04 | SARG | Specifically androgen-regulated gene protein |
| 1966 | Q9BW30 | TPPP3 | Tubulin polymerization-promoting protein family member 3 |
| 1967 | Q9BW91 | NUDT9 | ADP-ribose pyrophosphatase, mitochondrial |
| 1968 | Q9BWF3 | RBM4 | RNA-binding protein 4 |
| 1969 | Q9BWH2 | FUND2 | FUN14 domain-containing protein 2 |
| 1970 | Q9BWS9 | CHID1 | Chitinase domain-containing protein 1 |
| 1971 | Q9BWU0 | NADAP | Kanadaptin |
| 1972 | Q9BX40 | LS14B | Protein LSM14 homolog B |
| 1973 | Q9BX66 | SRBS1 | Sorbin and SH3 domain-containing protein 1 |
| 1974 | Q9BX68 | HINT2 | Histidine triad nucleotide-binding protein 2, mitochondrial |
| 1975 | Q9BX97 | PLVAP | Plasmalemma vesicle-associated protein |
| 1976 | Q9BXK5 | B2L13 | Bcl-2-like protein 13 |
| 1977 | Q9BXN1 | ASPN | Asporin |
| 1978 | Q9BXP5 | SRRT | Serrate RNA effector molecule homolog |
| 1979 | Q9BXV9 | CN142 | Uncharacterized protein C14orf142 |
| 1980 | Q9BY11 | PACN1 | Protein kinase C and casein kinase substrate in neurons protein 1 |
| 1981 | Q9BY42 | RTF2 | Protein RTF2 homolog |
| 1982 | Q9BY43 | CHM4A | Charged multi vesicular body protein 4a |
| 1983 | Q9BY44 | EIF2A | Eukaryotic translation initiation factor 2A; Eukaryotic translation initiation |
| | | | factor 2A, N-terminally processed |
| 1984 | Q9BY50 | SC11C | Signal peptidase complex catalytic subunit SEC11C |
| 1985 | Q9BY67 | CADM1 | Cell adhesion molecule 1 |
| 1986 | Q9BY77 | PDIP3 | Polymerase delta-interacting protein 3 |
| 1987 | Q9BY89 | K1671 | Uncharacterized protein KIAA1671 |
| 1988 | Q9BYT8 | NEUL | Neurolysin, mitochondrial |
| 1989 | Q9BZF9 | UACA | Uveal autoantigen with coiled-coil domains and ankyrin repeats |
| 1990 | Q9BZI7 | REN3B | Regulator of nonsense transcripts 3B |
| 1991 | Q9BZL4 | PP12C | Protein phosphatase 1 regulatory subunit 12C |
| 1992 | Q9BZQ8 | NIBAN | Protein Niban |
| 1993 | Q9BZV1 | UBXN6 | UBX domain-containing protein 6 |
| 1994 | Q9BZZ5 | API5 | Apoptosis inhibitor 5 |
| 1995 | Q9C005 | DPY30 | Protein dpy-30 homolog |
| 1996 | Q9C037 | TRIM4 | E3 ubiquitin-protein ligase TRIM4 |
| 1997 | Q9C0C2 | TB182 | 182 kDa tankyrase-1-binding protein |
| 1998 | Q9C0D0 | PHAR1 | Phosphatase and actin regulator 1 |
| 1999 | Q9C0H5 | RHG39 | Rho GTPase-activating protein 39 |
| 2000 | Q9C0J8 | WDR33 | pre-mRNA 3′ end processing protein WDR33 |
| 2001 | Q9GZM7 | TINAL | Tubulointerstitial nephritis antigen-like |
| 2002 | Q9GZT3 | SLIRP | SRA stem-loop-interacting RNA-binding protein, mitochondrial |
| 2003 | Q9GZV4 | IF5A2 | Eukaryotic translation initiation factor 5A-2 |
| 2004 | Q9GZZ9 | UBA5 | Ubiquitin-like modifier-activating enzyme 5 |
| 2005 | Q9H098 | F107B | Protein FAM107B |
| 2006 | Q9H0E2 | TOLIP | Toll-interacting protein |
| 2007 | Q9H0E9 | BRD8 | Bromodomain-containing protein 8 |
| 2008 | Q9H0N5 | PHS2 | Pterin-4-alpha-carbinolamine dehydratase 2 |
| 2009 | Q9H0R4 | HDHD2 | Haloacid dehalogenase-like hydrolase domain-containing protein 2 |
| 2010 | Q9H0U4 | RAB1B | Ras-related protein Rab-1B |
| 2011 | Q9H0W9 | CK054 | Ester hydrolase C11orf54 |
| 2012 | Q9H1B7 | I2BPL | Interferon regulatory factor 2-binding protein-like |
| 2013 | Q9H1C7 | CYTM1 | Cysteine-rich and transmembrane domain-containing protein 1 |
| 2014 | Q9H1E3 | NUCKS | Nuclear ubiquitous casein and cyclin-dependent kinase substrate 1 |
| 2015 | Q9H1K1 | ISCU | Iron-sulfur cluster assembly enzyme ISCU, mitochondrial |
| 2016 | Q9H223 | EHD4 | EH domain-containing protein 4 |
| 2017 | Q9H299 | SH3L3 | SH3 domain-binding glutamic acid-rich-like protein 3 |
| 2018 | Q9H2D6 | TARA | TRIO and F-actin-binding protein |
| 2019 | Q9H2G2 | SLK | STE20-like serine/threonine-protein kinase |
| 2020 | Q9H2K8 | TAOK3 | Serine/threonine-protein kinase TAO3 |
| 2021 | Q9H2U2 | IPYR2 | Inorganic pyrophosphatase 2, mitochondrial |
| 2022 | Q9H307 | PININ | Pinin |
| 2023 | Q9H3K6 | BOLA2 | BolA-like protein 2 |
| 2024 | Q9H3P7 | GCP60 | Golgi resident protein GCP60 |
| 2025 | Q9H3Q1 | BORG4 | Cdc42 effector protein 4 |
| 2026 | Q9H425 | CA198 | Uncharacterized protein C1orf198 |
| 2027 | Q9H444 | CHM4B | Charged multivesicular body protein 4b |
| 2028 | Q9H446 | RWDD1 | RWD domain-containing protein 1 |
| 2029 | Q9H479 | FN3K | Fructosamine-3-kinase |
| 2030 | Q9H4A3 | WNK1 | Serine/threonine-protein kinase WNK1 |
| 2031 | Q9H4A4 | AMPB | Aminopeptidase B |
| 2032 | Q9H4A5 | GLP3L | Golgi phosphoprotein 3-like |
| 2033 | Q9H4B7 | TBB1 | Tubulin beta-1 chain |
| 2034 | Q9H4G0 | E41L1 | Band 4.1-like protein 1 |
| 2035 | Q9H4I3 | TRABD | TraB domain-containing protein |
| 2036 | Q9H4X1 | RGCC | Regulator of cell cycle RGCC |
| 2037 | Q9H5N1 | RABE2 | Rab GTPase-binding effector protein 2 |
| 2038 | Q9H6F5 | CCD86 | Coiled-coil domain-containing protein 86 |
| 2039 | Q9H6N6 | MYH16 | Putative uncharacterized protein MYH16 |
| 2040 | Q9H6S3 | ES8L2 | Epidermal growth factor receptor kinase substrate 8-like protein 2 |
| 2041 | Q9H6Z4 | RANB3 | Ran-binding protein 3 |
| 2042 | Q9H6Z9 | EGLN3 | Egl nine homolog 3 |
| 2043 | Q9H7C9 | AAMDC | Mth938 domain-containing protein |
| 2044 | Q9H7L2 | KI3X1 | Putative killer cell immunoglobulin-like receptor-like protein KIR3DX1 |
| 2045 | Q9H7Z6 | KAT8 | Histone acetyltransferase KAT8 |
| 2046 | Q9H8H3 | MET7A | Methyltransferase-like protein 7A |
| 2047 | Q9H8L6 | MMRN2 | Multimerin-2 |
| 2048 | Q9H8Y8 | GORS2 | Golgi reassembly-stacking protein 2 |
| 2049 | Q9H910 | HN1L | Hematological and neurological expressed 1-like protein |
| 2050 | Q9H9E3 | COG4 | Conserved oligomeric Golgi complex subunit 4 |
| 2051 | Q9HA64 | KT3K | Ketosamine-3-kinase |
| 2052 | Q9HAB8 | PPCS | Phosphopantothenate-cysteine ligase |
| 2053 | Q9HAV0 | GBB4 | Guanine nucleotide-binding protein subunit beta-4 |
| 2054 | Q9HAV7 | GRPE1 | GrpE protein homolog 1, mitochondrial |
| 2055 | Q9HB71 | CYBP | Calcyclin-binding protein |
| 2056 | Q9HBL0 | TENS1 | Tensin-1 |
| 2057 | Q9HC35 | EMAL4 | Echinoderm microtubule-associated protein-like 4 |
| 2058 | Q9HC38 | GLOD4 | Glyoxalase domain-containing protein 4 |
| 2059 | Q9HC84 | MUC5B | Mucin-5B |
| 2060 | Q9HCB6 | SPON1 | Spondin-1 |
| 2061 | Q9HCC0 | MCCB | Methylcrotonoyl-CoA carboxylase beta chain, mitochondrial |
| 2062 | Q9HCG8 | CWC22 | Pre-mRNA-splicing factor CWC22 homolog |
| 2063 | Q9HCN8 | SDF2L | Stromal cell-derived factor 2-like protein 1 |
| 2064 | Q9HD42 | CHM1A | Charged multivesicular body protein 1a |
| 2065 | Q9HDC9 | APMAP | Adipocyte plasma membrane-associated protein |
| 2066 | Q9NNW7 | TRXR2 | Thioredoxin reductase 2, mitochondrial |
| 2067 | Q9NP61 | ARFG3 | ADP-ribosylation factor GTPase-activating protein 3 |
| 2068 | Q9NP74 | PALMD | Palmdelphin |
| 2069 | Q9NP79 | VTA1 | Vacuolar protein sorting-associated protein VTA1 homolog |
| 2070 | Q9NPF4 | OSGEP | Probable tRNA N6-adenosine threonylcarbamoyltransferase |
| 2071 | Q9NPJ3 | ACO13 | Acyl-coenzyme A thioesterase 13; Acyl-coenzyme A thioesterase 13, N- |
| | | | terminally processed |
| 2072 | Q9NQ50 | RM40 | 39S ribosomal protein L40, mitochondrial |
| 2073 | Q9NQ75 | CASS4 | Cas scaffolding protein family member 4 |
| 2074 | Q9NQC3 | RTN4 | Reticulon-4 |
| 2075 | Q9NQG5 | RPR1B | Regulation of nuclear pre-mRNA domain-containing protein 1B |
| 2076 | Q9NQR4 | NIT2 | Omega-amidase NIT2 |
| 2077 | Q9NQS1 | AVEN | Cell death regulator Aven |
| 2078 | Q9NQT5 | EXOS3 | Exosome complex component RRP40 |
| 2079 | Q9NQW7 | XPP1 | Xaa-Pro aminopeptidase 1 |
| 2080 | Q9NQX3 | GEPH | Gephyrin; Molybdopterin adenylyltransferase; Molybdopterin |
| | | | molybdenumtransferase |
| 2081 | Q9NQX4 | MYO5C | Unconventional myosin-Vc |
| 2082 | Q9NR12 | PDLI7 | PDZ and LIM domain protein 7 |
| 2083 | Q9NR28 | DBLOH | Diablo homolog, mitochondrial |
| 2084 | Q9NR30 | DDX21 | Nucleolar RNA helicase 2 |
| 2085 | Q9NR31 | SAR1A | GTP-binding protein SAR1a |
| 2086 | Q9NR45 | SIAS | Sialic acid synthase |
| 2087 | Q9NR46 | SHLB2 | Endophilin-B2 |
| 2088 | Q9NR56 | MBNL1 | Muscleblind-like protein 1 |
| 2089 | Q9NR96 | TLR9 | Toll-like receptor 9 |
| 2090 | Q9NRP2 | COXM2 | COX assembly mitochondrial protein 2 homolog |
| 2091 | Q9NRR5 | UBQL4 | Ubiquilin-4 |
| 2092 | Q9NRR8 | C42S1 | CDC42 small effector protein 1 |
| 2093 | Q9NRS6 | SNX15 | Sorting nexin-15 |
| 2094 | Q9NRV9 | HEBP1 | Heme-binding protein 1 |
| 2095 | Q9NRX4 | PHP14 | 14 kDa phosphohistidine phosphatase |
| 2096 | Q9NRY5 | F1142 | Protein FAM114A2 |
| 2097 | Q9NS71 | GKN1 | Gastrokine-1 |
| 2098 | Q9NS87 | KIF15 | Kinesin-like protein KIF15 |
| 2099 | Q9NSB8 | HOME2 | Homer protein homolog 2 |
| 2100 | Q9NSD9 | SYFB | Phenylalanine-tRNA ligase beta subunit |
| 2101 | Q9NSE4 | SYIM | Isoleucine-tRNA ligase, mitochondrial |
| 2102 | Q9NSK0 | KLC4 | Kinesin light chain 4 |
| 2103 | Q9NTK5 | OLA1 | Obg-like ATPase 1 |
| 2104 | Q9NTX5 | ECHD1 | Ethylmalonyl-CoA decarboxylase |
| 2105 | Q9NTZ6 | RBM12 | RNA-binding protein 12 |
| 2106 | Q9NUB1 | ACS2L | Acetyl-coenzyme A synthetase 2-like, mitochondrial |
| 2107 | Q9NUJ1 | ABHDA | Mycophenolic acid acyl-glucuronide esterase, mitochondrial |
| 2108 | Q9NUL5 | RYDEN | UPF0515 protein C19orf66 |
| 2109 | Q9NUP9 | LIN7C | Protein lin-7 homolog C |
| 2110 | Q9NUQ3 | TXLNG | Gamma-taxilin |
| 2111 | Q9NUQ6 | SPS2L | SPATS2-like protein |
| 2112 | Q9NUQ8 | ABCF3 | ATP-binding cassette sub-family F member 3 |
| 2113 | Q9NVA2 | 40787 | Septin-11 |
| 2114 | Q9NVI7 | ATD3A | ATPase family AAA domain-containing protein 3A |
| 2115 | Q9NVS9 | PNPO | Pyridoxine-5′-phosphate oxidase |
| 2116 | Q9NVU7 | SDA1 | Protein SDA1 homolog |
| 2117 | Q9NVZ3 | NECP2 | Adaptin ear-binding coat-associated protein 2 |
| 2118 | Q9NW68 | BSDC1 | BSD domain-containing protein 1 |
| 2119 | Q9NW97 | TMM51 | Transmembrane protein 51 |
| 2120 | Q9NWB6 | ARGL1 | Arginine and glutamate-rich protein 1 |
| 2121 | Q9NWH9 | SLTM | SAFB-like transcription modulator |
| 2122 | Q9NVW4 | CA123 | UPF0587 protein C1orf123 |
| 2123 | Q9NX24 | NHP2 | H/ACA ribonucleoprotein complex subunit 2 |
| 2124 | Q9NX40 | OCAD1 | OCIA domain-containing protein 1 |
| 2125 | Q9NX58 | LYAR | Cell growth-regulating nucleolar protein |
| 2126 | Q9NX63 | MIC19 | MICOS complex subunit MIC19 |
| 2127 | Q9NXG2 | THUM1 | THUMP domain-containing protein 1 |
| 2128 | Q9NXV6 | CARF | CDKN2A-interacting protein |
| 2129 | Q9NY12 | GAR1 | H/ACA ribonucleoprotein complex subunit 1 |
| 2130 | Q9NY27 | PP4R2 | Serine/threonine-protein phosphatase 4 regulatory subunit 2 |
| 2131 | Q9NY33 | DPP3 | Dipeptidyl peptidase 3 |
| 2132 | Q9NYB0 | TE2IP | Telomeric repeat-binding factor 2-interacting protein 1 |
| 2133 | Q9NYF8 | BCLF1 | Bcl-2-associated transcription factor 1 |
| 2134 | Q9NYJ1 | COA4 | Cytochrome c oxidase assembly factor 4 homolog, mitochondrial |
| 2135 | Q9NYL4 | FKB11 | Peptidyl-prolyl cis-trans isomerase FKBP11 |
| 2136 | Q9NYL9 | TMOD3 | Tropomodulin-3 |
| 2137 | Q9NYU2 | UGGG1 | UDP-glucose: glycoprotein glucosyltransferase 1 |
| 2138 | Q9NZ01 | TECR | Very-long-chain enoyl-CoA reductase |
| 2139 | Q9NZ45 | CISD1 | CDGSH iron-sulfur domain-containing protein 1 |
| 2140 | Q9NZB2 | F120A | Constitutive coactivator of PPAR-gamma-like protein 1 |
| 2141 | Q9NZL9 | MAT2B | Methionine adenosyltransferase 2 subunit beta |
| 2142 | Q9NZM3 | ITSN2 | Intersectin-2 |
| 2143 | Q9NZN4 | EHD2 | EH domain-containing protein 2 |
| 2144 | Q9NZR1 | TMOD2 | Tropomodulin-2 |
| 2145 | Q9NZU5 | LMCD1 | LIM and cysteine-rich domains protein 1 |
| 2146 | Q9NZV6 | MSRB1 | Methionine-R-sulfoxide reductase B1 |
| 2147 | Q9NZZ3 | CHMP5 | Charged multi vesicular body protein 5 |
| 2148 | Q9P013 | CWC15 | Spliceosome-associated protein CWC15 homolog |
| 2149 | Q9P016 | THYN1 | Thymocyte nuclear protein 1 |
| 2150 | Q9P0L0 | VAPA | Vesicle-associated membrane protein-associated protein A |
| 2151 | Q9P0M9 | RM27 | 39S ribosomal protein L27, mitochondrial |
| 2152 | Q9P0P8 | CF203 | Uncharacterized protein C6orf203 |
| 2153 | Q9P1F3 | ABRAL | Costars family protein ABRACL |
| 2154 | Q9P1Z2 | CACO1 | Calcium-binding and coiled-coil domain-containing protein 1 |
| 2155 | Q9P206 | K1522 | Uncharacterized protein KIAA1522 |
| 2156 | Q9P258 | RCC2 | Protein RCC2 |
| 2157 | Q9P265 | DIP2B | Disco-interacting protein 2 homolog B |
| 2158 | Q9P266 | JCAD | Junctional protein associated with coronary artery disease |
| 2159 | Q9P270 | SLAI2 | SLAIN motif-containing protein 2 |
| 2160 | Q9P289 | STK26 | Serine/threonine-protein kinase 26 |
| 2161 | Q9P2B2 | FPRP | Prostaglandin F2 receptor negative regulator |
| 2162 | Q9P2B4 | CT2NL | CTTNBP2 N-terminal-like protein |
| 2163 | Q9P2E9 | RRBP1 | Ribosome-binding protein 1 |
| 2164 | Q9P2F8 | SI1L2 | Signal-induced proliferation-associated 1-like protein 2 |
| 2165 | Q9P2J5 | SYLC | Leucine-tRNA ligase, cytoplasmic |
| 2166 | Q9P2K5 | MYEF2 | Myelin expression factor 2 |
| 2167 | Q9P2M7 | CING | Cingulin |
| 2168 | Q9P2R3 | ANFY1 | Rabankyrin-5 |
| 2169 | Q9P2R7 | SUCB1 | Succinyl-CoA ligase [ADP-forming] subunit beta, mitochondrial |
| 2170 | Q9P2T1 | GMPR2 | GMP reductase 2 |
| 2171 | Q9UBB5 | MBD2 | Methyl-CpG-binding domain protein 2 |
| 2172 | Q9UBC2 | EP15R | Epidermal growth factor receptor substrate 15-like 1 |
| 2173 | Q9UBI6 | GBG12 | Guanine nucleotide-binding protein G(I)/G(S)/G(O) subunit gamma-12 |
| 2174 | Q9UBQ7 | GRHPR | Glyoxylate reductase/hydroxypyruvate reductase |
| 2175 | Q9UBS4 | DJB11 | DnaJ homolog subfamily B member 11 |
| 2176 | Q9UBT2 | SAE2 | SUMO-activating enzyme subunit 2 |
| 2177 | Q9UBU3 | GHRL | Appetite-regulating hormone; Ghrelin-27; Ghrelin-28; Obestatin |
| 2178 | Q9UBV2 | SE1L1 | Protein sel-1 homolog 1 |
| 2179 | Q9UBW8 | CSN7A | COP9 signalosome complex subunit 7a |
| 2180 | Q9UBX5 | FBLN5 | Fibulin-5 |
| 2181 | Q9UBY9 | HSPB7 | Heat shock protein beta-7 |
| 2182 | Q9UD71 | PPR1B | Protein phosphatase 1 regulatory subunit 1B |
| 2183 | Q9UDT6 | CLIP2 | CAP-Gly domain-containing linker protein 2 |
| 2184 | Q9UDY2 | ZO2 | Tight junction protein ZO-2 |
| 2185 | Q9UDY4 | DNJB4 | DnaJ homolog subfamily B member 4 |
| 2186 | Q9UEE9 | CFDP1 | Craniofacial development protein 1 |
| 2187 | Q9UEW8 | STK39 | STE20/SPS1-related proline-alanine-rich protein kinase |
| 2188 | Q9UEY8 | ADDG | Gamma-adducin |
| 2189 | Q9UFG5 | CS025 | UPF0449 protein C19orf25 |
| 2190 | Q9UFW8 | CGBP1 | CGG triplet repeat-binding protein 1 |
| 2191 | Q9UGI8 | TES | Testin |
| 2192 | Q9UGM3 | DMBT1 | Deleted in malignant brain tumors 1 protein |
| 2193 | Q9UGP4 | LIMD1 | LIM domain-containing protein 1 |
| 2194 | Q9UGP8 | SEC63 | Translocation protein SEC63 homolog |
| 2195 | Q9UH65 | SWP70 | Switch-associated protein 70 |
| 2196 | Q9UHB6 | LIMA1 | LIM domain and actin-binding protein 1 |
| 2197 | Q9UHB9 | SRP68 | Signal recognition particle subunit SRP68 |
| 2198 | Q9UHD8 | 40057 | Septin-9 |
| 2199 | Q9UHD9 | UBQL2 | Ubiquilin-2 |
| 2200 | Q9UHG0 | DCDC2 | Doublecortin domain-containing protein 2 |
| 2201 | Q9UHG2 | PCSK1 | Big LEN; Big PEN-LEN; Big SAAS; KEP; Little LEN; Little |
| | | | SAAS; PEN; ProSAAS |
| 2202 | Q9UHG3 | PCYOX | Prenylcysteine oxidase 1 |
| 2203 | Q9UHR5 | S30BP | SAP30-binding protein |
| 2204 | Q9UHV9 | PFD2 | Prefoldin subunit 2 |
| 2205 | Q9UHX1 | PUF60 | Poly(U)-binding-splicing factor PUF60 |
| 2206 | Q9UI09 | NDUAC | NADH dehydrogenase [ubiquinone] 1 alpha subcomplex subunit 12 |
| 2207 | Q9UI10 | EI2BD | Translation initiation factor elF-2B subunit delta |
| 2208 | Q9UII2 | ATIF1 | ATPase inhibitor, mitochondrial |
| 2209 | Q9UIJ7 | KAD3 | GTP: AMP phosphotransferase AK3, mitochondrial |
| 2210 | Q9UIS9 | MBD1 | Methyl-CpG-binding domain protein 1 |
| 2211 | Q9UJ70 | NAGK | N-acetyl-D-glucosamine kinase |
| 2212 | Q9UJC3 | HOOK1 | Protein Hook homolog 1 |
| 2213 | Q9UJC5 | SH3L2 | SH3 domain-binding glutamic acid-rich-like protein 2 |
| 2214 | Q9UJU6 | DBNL | Drebrin-like protein |
| 2215 | Q9UJZ1 | STML2 | Stomatin-like protein 2, mitochondrial |
| 2216 | Q9UK22 | FBX2 | F-box only protein 2 |
| 2217 | Q9UK45 | LSM7 | U6 snRNA-associated Sm-like protein LSm7 |
| 2218 | Q9UK76 | HN1 | Hematological and neurological expressed 1 protein |
| 2219 | Q9UKG1 | DP13A | DCC-interacting protein 13-alpha |
| 2220 | Q9UKK9 | NUDT5 | ADP-sugar pyrophosphatase |
| 2221 | Q9UKM9 | RALY | RNA-binding protein Raly |
| 2222 | Q9UKS6 | PACN3 | Protein kinase C and casein kinase substrate in neurons protein 3 |
| 2223 | Q9UKV3 | ACINU | Apoptotic chromatin condensation inducer in the nucleus |
| 2224 | Q9UKX7 | NUP50 | Nuclear pore complex protein Nup50 |
| 2225 | Q9UKY7 | CDV3 | Protein CDV3 homolog |
| 2226 | Q9UL12 | SARDH | Sarcosine dehydrogenase, mitochondrial |
| 2227 | Q9UL46 | PSME2 | Proteasome activator complex subunit 2 |
| 2228 | Q9ULA0 | DNPEP | Aspartyl aminopeptidase |
| 2229 | Q9ULD2 | MTUS1 | Microtubule-associated tumor suppressor 1 |
| 2230 | Q9ULH0 | KDIS | Kinase D-interacting substrate of 220 kDa |
| 2231 | Q9ULP9 | TBC24 | TBC1 domain family member 24 |
| 2232 | Q9ULR0 | ISY1 | Pre-mRNA-splicing factor ISY1 homolog |
| 2233 | Q9ULR5 | PAI2B | Polyadenylate-binding protein-interacting protein 2B |
| 2234 | Q9ULV4 | COR1C | Coronin-1C |
| 2235 | Q9ULZ3 | ASC | Apoptosis-associated speck-like protein containing a CARD |
| 2236 | Q9UM54 | MYO6 | Unconventional myosin-VI |
| 2237 | Q9UMR2 | DD19B | ATP-dependent RNA helicase DDX19B |
| 2238 | Q9UMS0 | NFU1 | NFU1 iron-sulfur cluster scaffold homolog, mitochondrial |
| 2239 | Q9UMS4 | PRP19 | Pre-mRNA-processing factor 19 |
| 2240 | Q9UMS6 | SYNP2 | Synaptopodin-2 |
| 2241 | Q9UMX0 | UBQL1 | Ubiquilin-1 |
| 2242 | Q9UMX5 | NENF | Neudesin |
| 2243 | Q9UMY1 | NOL7 | Nucleolar protein 7 |
| 2244 | Q9UMY4 | SNX12 | Sorting nexin-12 |
| 2245 | Q9UMZ2 | SYNRG | Synergin gamma |
| 2246 | Q9UN36 | NDRG2 | Protein NDRG2 |
| 2247 | Q9UN37 | VPS4A | Vacuolar protein sorting-associated protein 4A |
| 2248 | Q9UN86 | G3BP2 | Ras GTPase-activating protein-binding protein 2 |
| 2249 | Q9UNF0 | PACN2 | Protein kinase C and casein kinase substrate in neurons protein 2 |
| 2250 | Q9UNF1 | MAGD2 | Melanoma-associated antigen D2 |
| 2251 | Q9UNL2 | SSRG | Translocon-associated protein subunit gamma |
| 2252 | Q9UNP9 | PPIE | Peptidyl-prolyl cis-trans isomerase E |
| 2253 | Q9UNS2 | CSN3 | COP9 signalosome complex subunit 3 |
| 2254 | Q9UNZ2 | NSF1C | NSFL1 cofactor p47 |
| 2255 | Q9UPN4 | CP131 | Centrosomal protein of 131 kDa |
| 2256 | Q9UPQ9 | TNR6B | Trinucleotide repeat-containing gene 6B protein |
| 2257 | Q9UPT8 | ZC3H4 | Zinc finger CCCH domain-containing protein 4 |
| 2258 | Q9UQ35 | SRRM2 | Serine/arginine repetitive matrix protein 2 |
| 2259 | Q9UQ80 | PA2G4 | Proliferation-associated protein 2G4 |
| 2260 | Q9UQB3 | CTND2 | Catenin delta-2 |
| 2261 | Q9UQE7 | SMC3 | Structural maintenance of chromosomes protein 3 |
| 2262 | Q9UQN3 | CHM2B | Charged multi vesicular body protein 2b |
| 2263 | Q9Y224 | CN166 | UPF0568 protein C14orf166 |
| 2264 | Q9Y230 | RUVB2 | RuvB-like 2 |
| 2265 | Q9Y237 | PIN4 | Peptidyl-prolyl cis-trans isomerase NIMA-interacting 4 |
| 2266 | Q9Y262 | EIF3L | Eukaryotic translation initiation factor 3 subunit L |
| 2267 | Q9Y265 | RUVB1 | RuvB-like 1 |
| 2268 | Q9Y266 | NUDC | Nuclear migration protein nudC |
| 2269 | Q9Y277 | VDAC3 | Voltage-dependent anion-selective channel protein 3 |
| 2270 | Q9Y281 | COF2 | Cofilin-2 |
| 2271 | Q9Y285 | SYFA | Phenylalanine-tRNA ligase alpha subunit |
| 2272 | Q9Y295 | DRG1 | Developmentally-regulated GTP-binding protein 1 |
| 2273 | Q9Y2B0 | CNPY2 | Protein canopy homolog 2 |
| 2274 | Q9Y2D5 | AKAP2 | A-kinase anchor protein 2 |
| 2275 | Q9Y2H0 | DLGP4 | Disks large-associated protein 4 |
| 2276 | Q9Y2H6 | FND3A | Fibronectin type-III domain-containing protein 3A |
| 2277 | Q9Y2I6 | NINL | Ninein-like protein |
| 2278 | Q9Y2I9 | TBC30 | TBC1 domain family member 30 |
| 2279 | Q9Y2J2 | E41L3 | Band 4.1-like protein 3; Band 4.1-like protein 3, N-terminally processed |
| 2280 | Q9Y2Q3 | GSTK1 | Glutathione S-transferase kappa 1 |
| 2281 | Q9Y2Q9 | RT28 | 28S ribosomal protein S28, mitochondrial |
| 2282 | Q9Y2S6 | TMA7 | Translation machinery-associated protein 7 |
| 2283 | Q9Y2U8 | MAN1 | Inner nuclear membrane protein Man1 |
| 2284 | Q9Y2V2 | CHSP1 | Calcium-regulated heat stable protein 1 |
| 2285 | Q9Y2W1 | TR150 | Thyroid hormone receptor-associated protein 3 |
| 2286 | Q9Y2W2 | WBP11 | WW domain-binding protein 11 |
| 2287 | Q9Y2X3 | NOP58 | Nucleolar protein 58 |
| 2288 | Q9Y2Z0 | SGT1 | Suppressor of G2 allele of SKP1 homolog |
| 2289 | Q9Y314 | NOSIP | Nitric oxide synthase-interacting protein |
| 2290 | Q9Y320 | TMX2 | Thioredoxin-related transmembrane protein 2 |
| 2291 | Q9Y333 | LSM2 | U6 snRNA-associated Sm-like protein LSm2 |
| 2292 | Q9Y365 | PCTL | PCTP-like protein |
| 2293 | Q9Y383 | LC7L2 | Putative RNA-binding protein Luc7-like 2 |
| 2294 | Q9Y3A5 | SBDS | Ribosome maturation protein SBDS |
| 2295 | Q9Y3B3 | TMED7 | Transmembrane emp24 domain-containing protein 7 |
| 2296 | Q9Y3B4 | SF3B6 | Splicing factor 3B subunit 6 |
| 2297 | Q9Y3B9 | RRP15 | RRP15-like protein |
| 2298 | Q9Y3C0 | CCD53 | WASH complex subunit CCDC53 |
| 2299 | Q9Y3C1 | NOP16 | Nucleolar protein 16 |
| 2300 | Q9Y3C6 | PPIL1 | Peptidyl-prolyl cis-trans isomerase-like 1 |
| 2301 | Q9Y3C8 | UFC1 | Ubiquitin-fold modifier-conjugating enzyme 1 |
| 2302 | Q9Y3D2 | MSRB2 | Methionine-R-sulfoxide reductase B2, mitochondrial |
| 2303 | Q9Y3D6 | FIS1 | Mitochondrial fission 1 protein |
| 2304 | Q9Y3E1 | HDGR3 | Hepatoma-derived growth factor-related protein 3 |
| 2305 | Q9Y3E7 | CHMP3 | Charged multi vesicular body protein 3 |
| 2306 | Q9Y3F4 | STRAP | Serine-threonine kinase receptor-associated protein |
| 2307 | Q9Y3I0 | RTCB | tRNA-splicing ligase RtcB homolog |
| 2308 | Q9Y3Q8 | T22D4 | TSC22 domain family protein 4 |
| 2309 | Q9Y3S1 | WNK2 | Serine/threonine-protein kinase WNK2 |
| 2310 | Q9Y3U8 | RL36 | 60S ribosomal protein L36 |
| 2311 | Q9Y3X0 | CCDC9 | Coiled-coil domain-containing protein 9 |
| 2312 | Q9Y3Z3 | SAMH1 | Deoxynucleoside triphosphate triphosphohydrolase SAMHD1 |
| 2313 | Q9Y446 | PKP3 | Plakophilin-3 |
| 2314 | Q9Y450 | HBS1L | HBS1-like protein |
| 2315 | Q9Y490 | TLN1 | Talin-1 |
| 2316 | Q9Y4F1 | FARP1 | FERM, RhoGEF and pleckstrin domain-containing protein 1 |
| 2317 | Q9Y4L1 | HYOU1 | Hypoxia up-regulated protein 1 |
| 2318 | Q9Y4P3 | TBL2 | Transducin beta-like protein 2 |
| 2319 | Q9Y4W6 | AFG32 | AFG3-like protein 2 |
| 2320 | Q9Y4Y9 | LSM5 | U6 snRNA-associated Sm-like protein LSm5 |
| 2321 | Q9Y4Z0 | LSM4 | U6 snRNA-associated Sm-like protein LSm4 |
| 2322 | Q9Y508 | RN114 | E3 ubiquitin-protein ligase RNF114 |
| 2323 | Q9Y520 | PRC2C | Protein PRRC2C |
| 2324 | Q9Y534 | CSDC2 | Cold shock domain-containing protein C2 |
| 2325 | Q9Y5F6 | PCDGM | Protocadherin gamma-C5 |
| 2326 | Q9Y5J1 | UTP18 | U3 small nucleolar RNA-associated protein 18 homolog |
| 2327 | Q9Y5J7 | TIM9 | Mitochondrial import inner membrane translocase subunit Tim9 |
| 2328 | Q9Y5J9 | TIM8B | Mitochondrial import inner membrane translocase subunit Tim8 B |
| 2329 | Q9Y5K6 | CD2AP | CD2-associated protein |
| 2330 | Q9Y5L4 | TIM13 | Mitochondrial import inner membrane translocase subunit Tim13 |
| 2331 | Q9Y5M8 | SRPRB | Signal recognition particle receptor subunit beta |
| 2332 | Q9Y5P6 | GMPPB | Mannose-1-phosphate guanyltransferase beta |
| 2333 | Q9Y5S9 | RBM8A | RNA-binding protein 8A |
| 2334 | Q9Y5U2 | TSSC4 | Protein TSSC4 |
| 2335 | Q9Y5V0 | ZN706 | Zinc finger protein 706 |
| 2336 | Q9Y5X1 | SNX9 | Sorting nexin-9 |
| 2337 | Q9Y5Y2 | NUBP2 | Cytosolic Fe-S cluster assembly factor NUBP2 |
| 2338 | Q9Y5Z4 | HEBP2 | Heme-binding protein 2 |
| 2339 | Q9Y608 | LRRF2 | Leucine-rich repeat flightless-interacting protein 2 |
| 2340 | Q9Y617 | SERC | Phosphoserine aminotransferase |
| 2341 | Q9Y639 | NPTN | Neuroplastin |
| 2342 | Q9Y657 | SPIN1 | Spindlin-1 |
| 2343 | Q9Y673 | ALG5 | Dolichyl-phosphate beta-glucosyltransferase |
| 2344 | Q9Y678 | COPG1 | Coatomer subunit gamma-1 |
| 2345 | Q9Y696 | CLIC4 | Chloride intracellular channel protein 4 |
| 2346 | Q9Y6C2 | EMIL1 | EMILIN-1 |
| 2347 | Q9Y6D5 | BIG2 | Brefeldin A-inhibited guanine nucleotide-exchange protein 2 |
| 2348 | Q9Y6E0 | STK24 | Serine/threonine-protein kinase 24; Serine/threonine-protein kinase 24 12 |
| | | | kDa subunit |
| 2349 | Q9Y6H1 | CHCH2 | Coiled-coil-helix-coiled-coil-helix domain-containing protein 2 |
| 2350 | Q9Y6I3 | EPN1 | Epsin-1 |
| 2351 | Q9Y6N5 | SQRD | Sulfide: quinone oxidoreductase, mitochondrial |
| 2352 | Q9Y6N6 | LAMC3 | Laminin subunit gamma-3 |
| 2353 | Q9Y6W5 | WASF2 | Wiskott-Aldrich syndrome protein family member 2 |
| 2354 | Q9Y6X8 | ZHX2 | Zinc fingers and homeoboxes protein 2 |
| 2355 | A1X283 | SPD2B | SH3 and PX domain-containing protein 2B |
| 2356 | B9A064 | IGLL5 | Immunoglobulin lambda-like polypeptide 5 |
| 2357 | C4AMC7 | WASH3 | Putative WAS protein family homolog 3 |
| 2358 | O00182 | LEG9 | Galectin-9 |
| 2359 | O00231 | PSD11 | 26S proteasome non-ATPase regulatory subunit 11 |
| 2360 | O00232 | PSD12 | 26S proteasome non-ATPase regulatory subunit 12 |
| 2361 | O00410 | IPO5 | Importin-5 |
| 2362 | O00429 | DNM1L | Dynamin-1-like protein |
| 2363 | O00461 | GOLI4 | Golgi integral membrane protein 4 |
| 2364 | O00488 | ZN593 | Zinc finger protein 593 |
| 2365 | O00584 | RNT2 | Ribonuclease T2 |
| 2366 | O00585 | CCL21 | C-C motif chemokine 21 |
| 2367 | O00592 | PODXL | Podocalyxin |
| 2368 | O00763 | ACACB | Acetyl-CoA carboxylase 2; Biotin carboxylase |
| 2369 | O14498 | ISLR | Immunoglobulin superfamily containing leucine-rich repeat protein |
| 2370 | O14787 | TNPO2 | Transportin-2 |
| 2371 | O15078 | CE290 | Centrosomal protein of 290 kDa |
| 2372 | O15117 | FYB | FYN-binding protein |
| 2373 | O15195 | VILL | Villin-like protein |
| 2374 | O15533 | TPSN | Tapasin |
| 2375 | O43164 | PJA2 | E3 ubiquitin-protein ligase Praja-2 |
| 2376 | O43169 | CYB5B | Cytochrome b5 type B |
| 2377 | O43294 | TGFI1 | Transforming growth factor beta-1-induced transcript 1 protein |
| 2378 | O43488 | ARK72 | Aflatoxin B1 aldehyde reductase member 2 |
| 2379 | O43516 | WIPF1 | WAS/WASL-interacting protein family member 1 |
| 2380 | O43681 | ASNA | ATPase ASNA1 |
| 2381 | O43747 | AP1G1 | AP-1 complex subunit gamma-1 |
| 2382 | O43866 | CD5L | CD5 antigen-like |
| 2383 | O60234 | GMFG | Glia maturation factor gamma |
| 2384 | O60361 | NDK8 | Putative nucleoside diphosphate kinase |
| 2385 | O60516 | 4EBP3 | Eukaryotic translation initiation factor 4E-binding protein 3 |
| 2386 | O60568 | PLOD3 | Procollagen-lysine, 2-oxoglutarate 5-dioxygenase 3 |
| 2387 | O75056 | SDC3 | Syndecan-3 |
| 2388 | O75131 | CPNE3 | Copine-3 |
| 2389 | O75251 | NDUS7 | NADH dehydrogenase [ubiquinone] iron-sulfur protein 7, mitochondrial |
| 2390 | O75306 | NDUS2 | NADH dehydrogenase [ubiquinone] iron-sulfur protein 2, mitochondrial |
| 2391 | O75323 | NIPS2 | Protein NipSnap homolog 2 |
| 2392 | O75339 | CILP1 | Cartilage intermediate layer protein 1; Cartilage intermediate layer protein |
| | | | 1 C1 |
| 2393 | O75363 | BCAS1 | Breast carcinoma-amplified sequence 1 |
| 2394 | O75436 | VP26A | Vacuolar protein sorting-associated protein 26A |
| 2395 | O75506 | HSBP1 | Heat shock factor-binding protein 1 |
| 2396 | O75663 | TIPRL | TIP41-like protein |
| 2397 | O75915 | PRAF3 | PRA1 family protein 3 |
| 2398 | O75964 | ATP5L | ATP synthase subunit g, mitochondrial |
| 2399 | O75995 | SASH3 | SAM and SH3 domain-containing protein 3 |
| 2400 | O94776 | MTA2 | Metastasis-associated protein MTA2 |
| 2401 | O94804 | STK10 | Serine/threonine-protein kinase 10 |
| 2402 | O94826 | TOM70 | Mitochondrial import receptor subunit TOM70 |
| 2403 | O94907 | DKK1 | Dickkopf-related protein 1 |
| 2404 | O95084 | PRS23 | Serine protease 23 |
| 2405 | O95171 | SCEL | Sciellin |
| 2406 | O95232 | LC7L3 | Luc7-like protein 3 |
| 2407 | O95299 | NDUAA | NADH dehydrogenase [ubiquinone] 1 alpha subcomplex subunit 10, |
| | | | mitochondrial |
| 2408 | O95302 | FKBP9 | Peptidyl-prolyl cis-trans isomerase FKBP9 |
| 2409 | O95340 | PAPS2 | Adenylyl-sulfate kinase; Bifunctional 3′-phosphoadenosine |
| | | | 5′-phosphosulfate synthase 2 |
| 2410 | O95571 | ETHE1 | Persulfide dioxygenase ETHE1, mitochondrial |
| 2411 | O95633 | FSTL3 | Follistatin-related protein 3 |
| 2412 | O95758 | PTBP3 | Polypyrimidine tract-binding protein 3 |
| 2413 | O95782 | AP2A1 | AP-2 complex subunit alpha-1 |
| 2414 | O95861 | BPNT1 | 3′(2′),5′-bisphosphate nucleotidase 1 |
| 2415 | O95965 | ITGBL | Integrin beta-like protein 1 |
| 2416 | O95967 | FBLN4 | EGF-containing fibulin-like extracellular matrix protein 2 |
| 2417 | O95999 | BCL10 | B-cell lymphoma/leukemia 10 |
| 2418 | P00403 | COX2 | Cytochrome c oxidase subunit 2 |
| 2419 | P00488 | F13A | Coagulation factor XIII A chain |
| 2420 | P00491 | PNPH | Purine nucleoside phosphorylase |
| 2421 | P00736 | C1R | Complement C1r subcomponent; Complement C1r subcomponent heavy |
| | | | chain |
| 2422 | P00742 | FA10 | Activated factor Xa heavy chain; Coagulation factor X; Factor X heavy |
| | | | chain; Factor X light chain |
| 2423 | P01019 | ANGT | Angiotensin 1-4; Angiotensin 1-5; Angiotensin 1-7; Angiotensin 1- |
| | | | 9; Angiotensin-1; Angiotensin-2; Angiotensin-3 |
| 2424 | P01031 | CO5 | C5a anaphylatoxin; Complement C5; Complement C5 alpha |
| | | | chain; Complement C5 alpha′ chain |
| 2425 | P01033 | TIMP1 | Metalloproteinase inhibitor 1 |
| 2426 | P01593 | KV101 | Ig kappa chain V-I region AG |
| 2427 | P01608 | KV116 | Ig kappa chain V-I region Roy |
| 2428 | P01613 | KV121 | Ig kappa chain V-I region Ni |
| 2429 | P01616 | KV203 | Ig kappa chain V-II region MIL |
| 2430 | P01617 | KV204 | Ig kappa chain V-II region TEW |
| 2431 | P01625 | KV402 | Ig kappa chain V-IV region Len |
| 2432 | P01700 | LV102 | Ig lambda chain V-I region HA |
| 2433 | P01701 | LV103 | Ig lambda chain V-I region NEW |
| 2434 | P01702 | LV104 | Ig lambda chain V-I region NIG-64 |
| 2435 | P01703 | LV105 | Ig lambda chain V-I region NEWM |
| 2436 | P01707 | LV204 | Ig lambda chain V-II region TRO |
| 2437 | P01714 | LV301 | Ig lambda chain V-III region SH |
| 2438 | P01717 | LV403 | Ig lambda chain V-IV region Hil |
| 2439 | P01743 | HV102 | Ig heavy chain V-I region HG3 |
| 2440 | P01764 | HV303 | Ig heavy chain V-III region 23 |
| 2441 | P01772 | HV311 | Ig heavy chain V-III region KOL |
| 2442 | P01781 | HV320 | Ig heavy chain V-III region GAL |
| 2443 | P01871 | IGHM | Ig mu chain C region |
| 2444 | P01877 | IGHA2 | Ig alpha-2 chain C region |
| 2445 | P01903 | DRA | HLA class II histocompatibility antigen, DR alpha chain |
| 2446 | P01912 | 2B13 | HLA class II histocompatibility antigen, DRB1-3 chain |
| 2447 | P02458 | CO2A1 | Chondrocalcin; Collagen alpha-1(II) chain |
| 2448 | P02538 | K2C6A | Keratin, type II cytoskeletal 6A |
| 2449 | P02549 | SPTA1 | Spectrin alpha chain, erythrocytic 1 |
| 2450 | P02649 | APOE | Apolipoprotein E |
| 2451 | P02654 | APOC1 | Apolipoprotein C-I; Truncated apolipoprotein C-I |
| 2452 | P02656 | APOC3 | Apolipoprotein C-III |
| 2453 | P02724 | GLPA | Glycophorin-A |
| 2454 | P02743 | SAMP | Serum amyloid P-component; Serum amyloid P-component(1-203) |
| 2455 | P02745 | C1QA | Complement C1q subcomponent subunit A |
| 2456 | P02746 | C1QB | Complement C1q subcomponent subunit B |
| 2457 | P02747 | C1QC | Complement C1q subcomponent subunit C |
| 2458 | P02753 | RET4 | Plasma retinol-binding protein(1-176); Plasma retinol-binding |
| | | | protein(1-179); Plasma retinol-binding protein(1-181) |
| 2459 | P02775 | CXCL7 | Beta-thromboglobulin; Connective tissue-activating peptide III; Connective |
| | | | tissue-activating peptide III(1-81) |
| 2460 | P02776 | PLF4 | Platelet factor 4; Platelet factor 4, short form |
| 2461 | P02792 | FRIL | Ferritin light chain |
| 2462 | P03973 | SLPI | Antileukoproteinase |
| 2463 | P04003 | C4BPA | C4b-binding protein alpha chain |
| 2464 | P04114 | APOB | Apolipoprotein B-100; Apolipoprotein B-48 |
| 2465 | P04150 | GCR | Glucocorticoid receptor |
| 2466 | P04181 | OAT | Ornithine aminotransferase, hepatic form; Ornithine aminotransferase, |
| | | | mitochondrial |
| 2467 | P04208 | LV106 | Ig lambda chain V-I region WAH |
| 2468 | P04211 | LV001 | Ig lambda chain V region 4A |
| 2469 | P04216 | THY1 | Thy-1 membrane glycoprotein |
| 2470 | P04233 | HG2A | HLA class II histocompatibility antigen gamma chain |
| 2471 | P04271 | S100B | Protein S100-B |
| 2472 | P04275 | VWF | von Willebrand antigen 2; von Willebrand factor |
| 2473 | P04430 | KV122 | Ig kappa chain V-I region BAN |
| 2474 | P04433 | KV309 | Ig kappa chain V-III region VG |
| 2475 | P04434 | KV310 | Ig kappa chain V-III region VH |
| 2476 | P04899 | GNAI2 | Guanine nucleotide-binding protein G(i) subunit alpha-2 |
| 2477 | P05019 | IGF1 | Insulin-like growth factor I |
| 2478 | P05067 | A4 | Amyloid beta A4 protein; Beta-amyloid protein 40; Beta-amyloid protein |
| | | | 42; C31; C80; C83; C99 |
| 2479 | P05090 | APOD | Apolipoprotein D |
| 2480 | P05107 | ITB2 | Integrin beta-2 |
| 2481 | P05155 | IC1 | Plasma protease C1 inhibitor |
| 2482 | P05156 | CFAI | Complement factor I ; Complement factor I heavy chain; Complement factor |
| | | | I light chain |
| 2483 | P05452 | TETN | Tetranectin |
| 2484 | P05546 | HEP2 | Heparin cofactor 2 |
| 2485 | P06727 | APOA4 | Apolipoprotein A-IV |
| 2486 | P06731 | CEAM5 | Carcinoembryonic antigen-related cell adhesion molecule 5 |
| 2487 | P06737 | PYGL | Glycogen phosphorylase, liver form |
| 2488 | P06756 | ITAV | Integrin alpha-V; Integrin alpha-V heavy chain; lntegrin alpha-V light chain |
| 2489 | P06865 | HEXA | Beta-hexosaminidase subunit alpha |
| 2490 | P06889 | LV405 | Ig lambda chain V-IV region MOL |
| 2491 | P06899 | H2B1J | Histone H2B type 1-J |
| 2492 | P07225 | PROS | Vitamin K-dependent protein S |
| 2493 | P07357 | CO8A | Complement component C8 alpha chain |
| 2494 | P07384 | CAN1 | Calpain-1 catalytic subunit |
| 2495 | P07451 | CAH3 | Carbonic anhydrase 3 |
| 2496 | P07476 | INVO | Involucrin |
| 2497 | P07686 | HEXB | Beta-hexosaminidase subunit beta; Beta-hexosaminidase subunit beta |
| | | | chain A |
| 2498 | P08138 | TNR16 | Tumor necrosis factor receptor superfamily member 16 |
| 2499 | P08236 | BGLR | Beta-glucuronidase |
| 2500 | P08253 | MMP2 | 72 kDa type IV collagenase; PEX |
| 2501 | P08493 | MGP | Matrix Gia protein |
| 2502 | P08519 | APOA | Apolipoprotein(a) |
| 2503 | P08567 | PLEK | Pleckstrin |
| 2504 | P08574 | CY1 | Cytochrome c1, heme protein, mitochondrial |
| 2505 | P08575 | PTPRC | Receptor-type tyrosine-protein phosphatase C |
| 2506 | P08603 | CFAH | Complement factor H |
| 2507 | P08637 | FCG3A | Low affinity immunoglobulin gamma Fc region receptor III-A |
| 2508 | P08697 | A2AP | Alpha-2-antiplasmin |
| 2509 | P08779 | K1C16 | Keratin, type I cytoskeletal 16 |
| 2510 | P09104 | ENOG | Gamma-enolase |
| 2511 | P09417 | DHPR | Dihydropteridine reductase |
| 2512 | P09486 | SPRC | SPARC |
| 2513 | P09543 | CN37 | 2′,3′-cyclic-nucleotide 3′-phosphodiesterase |
| 2514 | P09668 | CATH | Cathepsin H; Cathepsin H heavy chain; Cathepsin H light chain; Cathepsin |
| | | | H mini chain; Pro-cathepsin H |
| 2515 | P09871 | C1S | Complement C1s subcomponent; Complement C1s subcomponent heavy |
| | | | chain |
| 2516 | P0C0L5 | CO4B | C4a anaphylatoxin; C4b-B; C4d-B; Complement C4 beta |
| | | | chain; Complement C4 gamma chain; Complement C4-B |
| 2517 | P0C7T7 | FMAS1 | Putative uncharacterized protein FRMD6-AS1 |
| 2518 | P0CG05 | LAC2 | Ig lambda-2 chain C regions |
| 2519 | P0CG38 | POTEI | POTE ankyrin domain family member I |
| 2520 | P0DJI8 | SAA1 | Amyloid protein A; Serum amyloid A-1 protein; Serum amyloid protein A(2- |
| | | | 102); Serum amyloid protein A(2-103) |
| 2521 | P0DKV0 | S31C1 | Spermatogenesis-associated protein 31C1 |
| 2522 | P10145 | IL8 | IL-8(5-77); IL-8(6-77); IL-8(7-77); IL-8(8-77); IL-8(9-77); Interleukin- |
| | | | 8; MDNCF-a |
| 2523 | P10153 | RNAS2 | Non-secretory ribonuclease |
| 2524 | P10155 | RO60 | 60 kDa SS-A/Ro ribonucleoprotein |
| 2525 | P10301 | RRAS | Ras-related protein R-Ras |
| 2526 | P10619 | PPGB | Lysosomal protective protein; Lysosomal protective protein 20 kDa |
| | | | chain; Lysosomal protective protein 32 kDa chain |
| 2527 | P10644 | KAP0 | cAMP-dependent protein kinase type I-alpha regulatory subunit |
| 2528 | P11215 | ITAM | Integrin alpha-M |
| 2529 | P11216 | PYGB | Glycogen phosphorylase, brain form |
| 2530 | P11233 | RALA | Ras-related protein Ral-A |
| 2531 | P11277 | SPTB1 | Spectrin beta chain, erythrocytic |
| 2532 | P11413 | G6PD | Glucose-6-phosphate 1-dehydrogenase |
| 2533 | P11498 | PYC | Pyruvate carboxylase, mitochondrial |
| 2534 | P11678 | PERE | Eosinophil peroxidase; Eosinophil peroxidase heavy chain; Eosinophil |
| | | | peroxidase light chain |
| 2535 | P12532 | KCRU | Creatine kinase U-type, mitochondrial |
| 2536 | P12724 | ECP | Eosinophil cationic protein |
| 2537 | P12829 | MYL4 | Myosin light chain 4 |
| 2538 | P12955 | PEPD | Xaa-Pro dipeptidase |
| 2539 | P13611 | CSPG2 | Versican core protein |
| 2540 | P13671 | CO6 | Complement component C6 |
| 2541 | P13674 | P4HA1 | Prolyl 4-hydroxylase subunit alpha-1 |
| 2542 | P13688 | CEAM1 | Carcinoembryonic antigen-related cell adhesion molecule 1 |
| 2543 | P13727 | PRG2 | Bone marrow proteoglycan; Eosinophil granule major basic protein |
| 2544 | P13760 | 2B14 | HLA class II histocompatibility antigen, DRB1-4 beta chain |
| 2545 | P13798 | ACPH | Acylamino-acid-releasing enzyme |
| 2546 | P13942 | COBA2 | Collagen alpha-2(XI) chain |
| 2547 | P14151 | LYAM1 | L-selectin |
| 2548 | P14780 | MMP9 | 67 kDa matrix metalloproteinase-9; 82 kDa matrix metalloproteinase- |
| | | | 9; Matrix metalloproteinase-9 |
| 2549 | P15121 | ALDR | Aldose reductase |
| 2550 | P15153 | RAC2 | Ras-related C3 botulinum toxin substrate 2 |
| 2551 | P15408 | FOSL2 | Fos-related antigen 2 |
| 2552 | P15502 | ELN | Elastin |
| 2553 | P15586 | GNS | N-acetylglucosamine-6-sulfatase |
| 2554 | P15692 | VEGFA | Vascular endothelial growth factor A |
| 2555 | P15941 | MUC1 | Mucin-1; Mucin-1 subunit alpha; Mucin-1 subunit beta |
| 2556 | P16035 | TIMP2 | Metalloproteinase inhibitor 2 |
| 2557 | P16157 | ANK1 | Ankyrin-1 |
| 2558 | P16278 | BGAL | Beta-galactosidase |
| 2559 | P16671 | CD36 | Platelet glycoprotein 4 |
| 2560 | P17050 | NAGAB | Alpha-N-acetylgalactosaminidase |
| 2561 | P17174 | AATC | Aspartate aminotransferase, cytoplasmic |
| 2562 | P17275 | JUNB | Transcription factor jun-B |
| 2563 | P17858 | PFKAL | ATP-dependent 6-phosphofructokinase, liver type |
| 2564 | P17936 | IBP3 | Insulin-like growth factor-binding protein 3 |
| 2565 | P18428 | LBP | Lipopolysaccharide-binding protein |
| 2566 | P19075 | TSN8 | Tetraspanin-8 |
| 2567 | P19256 | LFA3 | Lymphocyte function-associated antigen 3 |
| 2568 | P19367 | HXK1 | Hexokinase-1 |
| 2569 | P19827 | ITIH1 | Inter-alpha-trypsin inhibitor heavy chain H1 |
| 2570 | P19838 | NFKB1 | Nuclear factor NF-kappa-B p105 subunit; Nuclear factor NF-kappa-B p50 |
| | | | subunit |
| 2571 | P19971 | TYPH | Thymidine phosphorylase |
| 2572 | P20036 | DPA1 | HLA class II histocompatibility antigen, DP alpha 1 chain |
| 2573 | P20039 | 2B1B | HLA class II histocompatibility antigen, DRB1-11 beta chain |
| 2574 | P20073 | ANXA7 | Annexin A7 |
| 2575 | P20851 | C4BPB | C4b-binding protein beta chain |
| 2576 | P20908 | CO5A1 | Collagen alpha-1(V) chain |
| 2577 | P21283 | VATC1 | V-type proton ATPase subunit C 1 |
| 2578 | P21397 | AOFA | Amine oxidase [flavin-containing] A |
| 2579 | P21741 | MK | Midkine |
| 2580 | P22234 | PUR6 | Multifunctional protein ADE2; Phosphoribosylaminoimidazole carboxylase |
| 2581 | P22352 | GPX3 | Glutathione peroxidase 3 |
| 2582 | P22676 | CALB2 | Calretinin |
| 2583 | P22692 | IBP4 | Insulin-like growth factor-binding protein 4 |
| 2584 | P22897 | MRC1 | Macrophage mannose receptor 1 |
| 2585 | P23083 | HV103 | Ig heavy chain V-I region V35 |
| 2586 | P23141 | EST1 | Liver carboxylesterase 1 |
| 2587 | P23368 | MAOM | NAD-dependent malic enzyme, mitochondrial |
| 2588 | P24158 | PRTN3 | Myeloblastin |
| 2589 | P24592 | IBP6 | Insulin-like growth factor-binding protein 6 |
| 2590 | P25774 | CATS | Cathepsin S |
| 2591 | P26440 | IVD | Isovaleryl-CoA dehydrogenase, mitochondrial |
| 2592 | P27105 | STOM | Erythrocyte band 7 integral membrane protein |
| 2593 | P27169 | PON1 | Serum paraoxonase/arylesterase 1 |
| 2594 | P27338 | AOFB | Amine oxidase [flavin-containing] B |
| 2595 | P27658 | CO8A1 | Collagen alpha-1(VIII) chain; Vastatin |
| 2596 | P27918 | PROP | Properdin |
| 2597 | P28062 | PSB8 | Proteasome subunit beta type-8 |
| 2598 | P28065 | PSB9 | Proteasome subunit beta type-9 |
| 2599 | P28300 | LYOX | Protein-lysine 6-oxidase |
| 2600 | P28482 | MK01 | Mitogen-activated protein kinase 1 |
| 2601 | P28676 | GRAN | Grancalcin |
| 2602 | P29120 | NEC1 | Neuroendocrine convertase 1 |
| 2603 | P29218 | IMPA1 | Inositol monophosphatase 1 |
| 2604 | P29350 | PTN6 | Tyrosine-protein phosphatase non-receptor type 6 |
| 2605 | P29373 | RABP2 | Cellular retinoic acid-binding protein 2 |
| 2606 | P29466 | CASP1 | Caspase-1; Caspase-1 subunit p10; Caspase-1 subunit p20 |
| 2607 | P30153 | 2AAA | Serine/threonine-protein phosphatase 2A 65 kDa regulatory subunit A |
| | | | alpha isoform |
| 2608 | P30613 | KPYR | Pyruvate kinase PKLR |
| 2609 | P31323 | KAP3 | cAMP-dependent protein kinase type II-beta regulatory subunit |
| 2610 | P31431 | SDC4 | Syndecan-4 |
| 2611 | P31947 | 1433S | 14-3-3 protein sigma |
| 2612 | P32320 | CDD | Cytidine deaminase |
| 2613 | P33121 | ACSL1 | Long-chain-fatty-acid-CoA ligase 1 |
| 2614 | P34949 | MPI | Mannose-6-phosphate isomerase |
| 2615 | P35442 | TSP2 | Thrombospondin-2 |
| 2616 | P35558 | PCKGC | Phosphoenolpyruvate carboxykinase, cytosolic [GTP] |
| 2617 | P35900 | K1C20 | Keratin, type I cytoskeletal 20 |
| 2618 | P36543 | VATE1 | V-type proton ATPase subunit E 1 |
| 2619 | P36952 | SPB5 | Serpin B5 |
| 2620 | P36955 | PEDF | Pigment epithelium-derived factor |
| 2621 | P36969 | GPX4 | Phospholipid hydroperoxide glutathione peroxidase, mitochondrial |
| 2622 | P38570 | ITAE | Integrin alpha-E; Integrin alpha-E heavy chain; Integrin alpha-E light chain |
| 2623 | P40199 | CEAM6 | Carcinoembryonic antigen-related cell adhesion molecule 6 |
| 2624 | P40261 | NNMT | Nicotinamide N-methyltransferase |
| 2625 | P40306 | PSB10 | Proteasome subunit beta type-10 |
| 2626 | P40763 | STAT3 | Signal transducer and activator of transcription 3 |
| 2627 | P41208 | CETN2 | Centrin-2 |
| 2628 | P41218 | MNDA | Myeloid cell nuclear differentiation antigen |
| 2629 | P41222 | PTGDS | Prostaglandin-H2 D-isomerase |
| 2630 | P41240 | CSK | Tyrosine-protein kinase CSK |
| 2631 | P42224 | STAT1 | Signal transducer and activator of transcription 1-alpha/beta |
| 2632 | P42574 | CASP3 | Caspase-3; Caspase-3 subunit p12; Caspase-3 subunit p17 |
| 2633 | P42768 | WASP | Wiskott-Aldrich syndrome protein |
| 2634 | P43490 | NAMPT | Nicotinamide phosphoribosyltransferase |
| 2635 | P43652 | AFAM | Afamin |
| 2636 | P43681 | ACHA4 | Neuronal acetylcholine receptor subunit alpha-4 |
| 2637 | P45877 | PPIC | Peptidyl-prolyl cis-trans isomerase C |
| 2638 | P46063 | RECQ1 | ATP-dependent DNA helicase Q1 |
| 2639 | P47755 | CAZA2 | F-actin-capping protein subunit alpha-2 |
| 2640 | P48059 | LIMS1 | LIM and senescent cell antigen-like-containing domain protein 1 |
| 2641 | P48147 | PPCE | Prolyl endopeptidase |
| 2642 | P48556 | PSMD8 | 26S proteasome non-ATPase regulatory subunit 8 |
| 2643 | P49747 | COMP | Cartilage oligomeric matrix protein |
| 2644 | P49908 | SEPP1 | Selenoprotein P |
| 2645 | P49959 | MRE11 | Double-strand break repair protein MRE11A |
| 2646 | P50281 | MMP14 | Matrix metalloproteinase-14 |
| 2647 | P50453 | SPB9 | Serpin B9 |
| 2648 | P50570 | DYN2 | Dynamin-2 |
| 2649 | P51148 | RAB5C | Ras-related protein Rab-5C |
| 2650 | P51149 | RAB7A | Ras-related protein Rab-7a |
| 2651 | P51513 | NOVA1 | RNA-binding protein Nova-1 |
| 2652 | P51659 | DHB4 | (3R)-hydroxyacyl-CoA dehydrogenase; Enoyl-CoA hydratase |
| | | | 2; Peroxisomal multifunctional enzyme type 2 |
| 2653 | P52294 | IMA5 | Importin subunit alpha-5; Importin subunit alpha-5, N-terminally processed |
| 2654 | P52926 | HMGA2 | High mobility group protein HMGI-C |
| 2655 | P53004 | BIEA | Biliverdin reductase A |
| 2656 | P53634 | CATC | Dipeptidyl peptidase 1; Dipeptidyl peptidase 1 exclusion domain chain |
| 2657 | P53804 | TTC3 | E3 ubiquitin-protein ligase TTC3 |
| 2658 | P54821 | PRRX1 | Paired mesoderm homeobox protein 1 |
| 2659 | P55001 | MFAP2 | Microfibrillar-associated protein 2 |
| 2660 | P55083 | MFAP4 | Microfibril-associated glycoprotein 4 |
| 2661 | P55786 | PSA | Puromycin-sensitive aminopeptidase |
| 2662 | P55795 | HNRH2 | Heterogeneous nuclear ribonucleoprotein H2 |
| 2663 | P55809 | SCOT1 | Succinyl-CoA: 3-ketoacid coenzyme A transferase 1, mitochondrial |
| 2664 | P55854 | SUMO3 | Small ubiquitin-related modifier 3 |
| 2665 | P56181 | NDUV3 | NADH dehydrogenase [ubiquinone] flavoprotein 3, mitochondrial |
| 2666 | P56470 | LEG4 | Galectin-4 |
| 2667 | P60033 | CD81 | CD81 antigen |
| 2668 | P60891 | PRPS1 | Ribose-phosphate pyrophosphokinase 1 |
| 2669 | P61254 | RL26 | 60S ribosomal protein L26 |
| 2670 | P61421 | VA0D1 | V-type proton ATPase subunit d 1 |
| 2671 | P61586 | RHOA | Transforming protein RhoA |
| 2672 | P61923 | COPZ1 | Coatomer subunit zeta-1 |
| 2673 | P61970 | NTF2 | Nuclear transport factor 2 |
| 2674 | P62495 | ERF1 | Eukaryotic peptide chain release factor subunit 1 |
| 2675 | P62736 | ACTA | Actin, aortic smooth muscle |
| 2676 | P62834 | RAP1A | Ras-related protein Rap-1A |
| 2677 | P62877 | RBX1 | E3 ubiquitin-protein ligase RBX1; E3 ubiquitin-protein ligase RBX1, N- |
| | | | terminally processed |
| 2678 | P63000 | RAC1 | Ras-related C3 botulinum toxin substrate 1 |
| 2679 | P63010 | AP2B1 | AP-2 complex subunit beta |
| 2680 | P63098 | CANB1 | Calcineurin subunit B type 1 |
| 2681 | P68104 | EF1A1 | Elongation factor 1-alpha 1 |
| 2682 | P78527 | PRKDC | DNA-dependent protein kinase catalytic subunit |
| 2683 | P78559 | MAP1A | MAPI light chain LC2; MAP1A heavy chain; Microtubule-associated |
| | | | protein 1A |
| 2684 | P80511 | S10AC | Calcitermin; Protein S100-A12 |
| 2685 | P83111 | LACTB | Serine beta-lactamase-like protein LACTB, mitochondrial |
| 2686 | P84085 | ARF5 | ADP-ribosylation factor 5 |
| 2687 | P84095 | RHOG | Rho-related GTP-binding protein RhoG |
| 2688 | P84157 | MXRA7 | Matrix-remodeling-associated protein 7 |
| 2689 | Q00765 | REEP5 | Receptor expression-enhancing protein 5 |
| 2690 | Q01484 | ANK2 | Ankyrin-2 |
| 2691 | Q01813 | PFKAP | ATP-dependent 6-phosphofructokinase, platelet type |
| 2692 | Q02318 | CP27A | Sterol 26-hydroxylase, mitochondrial |
| 2693 | Q02809 | PLOD1 | Procollagen-lysine,2-oxoglutarate 5-dioxygenase 1 |
| 2694 | Q02978 | M2OM | Mitochondrial 2-oxoglutarate/malate carrier protein |
| 2695 | Q02985 | FHR3 | Complement factor H-related protein 3 |
| 2696 | Q03001 | DYST | Dystonin |
| 2697 | Q03135 | CAV1 | Caveolin-1 |
| 2698 | Q03591 | FHR1 | Complement factor H-related protein 1 |
| 2699 | Q04446 | GLGB | 1,4-alpha-glucan-branching enzyme |
| 2700 | Q04695 | K1C17 | Keratin, type I cytoskeletal 17 |
| 2701 | Q04941 | PLP2 | Proteolipid protein 2 |
| 2702 | Q05469 | LIPS | Hormone-sensitive lipase |
| 2703 | Q06033 | ITIH3 | Inter-alpha-trypsin inhibitor heavy chain H3 |
| 2704 | Q06828 | FMOD | Fibromodulin |
| 2705 | Q07507 | DERM | Dermatopontin |
| 2706 | Q07812 | BAX | Apoptosis regulator BAX |
| 2707 | Q08209 | PP2BA | Serine/threonine-protein phosphatase 2B catalytic subunit alpha isoform |
| 2708 | Q08AM6 | VAC14 | Protein VAC14 homolog |
| 2709 | Q0VD83 | APOBR | Apolipoprotein B receptor |
| 2710 | Q10588 | BST1 | ADP-ribosyl cyclase/cyclic ADP-ribose hydrolase 2 |
| 2711 | Q12797 | ASPH | Aspartyl/asparaginyl beta-hydroxylase |
| 2712 | Q12882 | DPYD | Dihydropyrimidine dehydrogenase [NADP(+)] |
| 2713 | Q12905 | ILF2 | Interleukin enhancer-binding factor 2 |
| 2714 | Q12986 | NFX1 | Transcriptional repressor NF-X1 |
| 2715 | Q13045 | FLII | Protein flightless-1 homolog |
| 2716 | Q13136 | LIPA1 | Liprin-alpha-1 |
| 2717 | Q13308 | PTK7 | Inactive tyrosine-protein kinase 7 |
| 2718 | Q13418 | ILK | Integrin-linked protein kinase |
| 2719 | Q13421 | MSLN | Megakaryocyte-potentiating factor; Mesothelin; Mesothelin, cleaved form |
| 2720 | Q13464 | ROCK1 | Rho-associated protein kinase 1 |
| 2721 | Q13507 | TRPC3 | Short transient receptor potential channel 3 |
| 2722 | Q13509 | TBB3 | Tubulin beta-3 chain |
| 2723 | Q13522 | PPR1A | Protein phosphatase 1 regulatory subunit 1A |
| 2724 | Q13564 | ULA1 | NEDD8-activating enzyme E1 regulatory subunit |
| 2725 | Q13724 | MOGS | Mannosyl-oligosaccharide glucosidase |
| 2726 | Q13751 | LAMB3 | Laminin subunit beta-3 |
| 2727 | Q13753 | LAMC2 | Laminin subunit gamma-2 |
| 2728 | Q14002 | CEAM7 | Carcinoembryonic antigen-related cell adhesion molecule 7 |
| 2729 | Q14005 | IL16 | Interleukin-16; Pro-interleukin-16 |
| 2730 | Q14028 | CNGB1 | Cyclic nucleotide-gated cation channel beta-1 |
| 2731 | Q14108 | SCRB2 | Lysosome membrane protein 2 |
| 2732 | Q14116 | IL18 | Interleukin-18 |
| 2733 | Q14134 | TRI29 | Tripartite motif-containing protein 29 |
| 2734 | Q14141 | 38961 | Septin-6 |
| 2735 | Q14192 | FHL2 | Four and a half LIM domains protein 2 |
| 2736 | Q14194 | DPYL1 | Dihydropyrimidinase-related protein 1 |
| 2737 | Q14508 | WFDC2 | WAP four-disulfide core domain protein 2 |
| 2738 | Q14520 | HABP2 | Hyaluronan-binding protein 2; Hyaluronan-binding protein 2 27 kDa light |
| | | | chain |
| 2739 | Q14624 | ITIH4 | 35 kDa inter-alpha-trypsin inhibitor heavy chain H4; 70 kDa inter-alpha- |
| | | | trypsin inhibitor heavy chain H4 |
| 2740 | Q14839 | CHD4 | Chromodomain-helicase-DNA-binding protein 4 |
| 2741 | Q15008 | PSMD6 | 26S proteasome non-ATPase regulatory subunit 6 |
| 2742 | Q15036 | SNX17 | Sorting nexin-17 |
| 2743 | Q15113 | PCOC1 | Procollagen C-endopeptidase enhancer 1 |
| 2744 | Q15124 | PGM5 | Phosphoglucomutase-like protein 5 |
| 2745 | Q15257 | PTPA | Serine/threonine-protein phosphatase 2A activator |
| 2746 | Q15393 | SF3B3 | Splicing factor 3B subunit 3 |
| 2747 | Q15404 | RSU1 | Ras suppressor protein 1 |
| 2748 | Q15436 | SC23A | Protein transport protein Sec23A |
| 2749 | Q15811 | ITSN1 | Intersectin-1 |
| 2750 | Q16610 | ECM1 | Extracellular matrix protein 1 |
| 2751 | Q16626 | MEA1 | Male-enhanced antigen 1 |
| 2752 | Q16647 | PTGIS | Prostacyclin synthase |
| 2753 | Q16658 | FSCN1 | Fascin |
| 2754 | Q16769 | QPCT | Glutaminyl-peptide cyclotransferase |
| 2755 | Q16774 | KGUA | Guanylate kinase |
| 2756 | Q16787 | LAMA3 | Laminin subunit alpha-3 |
| 2757 | Q16799 | RTN1 | Reticulon-1 |
| 2758 | Q16853 | AOC3 | Membrane primary amine oxidase |
| 2759 | Q16881 | TRXR1 | Thioredoxin reductase 1, cytoplasmic |
| 2760 | Q17RS7 | GEN | Flap endonuclease GEN homolog 1 |
| 2761 | Q32P28 | P3H1 | Prolyl 3-hydroxylase 1 |
| 2762 | Q4V9L6 | TM119 | Transmembrane protein 119 |
| 2763 | Q4ZHG4 | FNDC1 | Fibronectin type III domain-containing protein 1 |
| 2764 | Q53FA7 | QORX | Quinone oxidoreductase PIG3 |
| 2765 | Q53H12 | AGK | Acylglycerol kinase, mitochondrial |
| 2766 | Q53QV2 | LBH | Protein LBH |
| 2767 | Q562R1 | ACTBL | Beta-actin-like protein 2 |
| 2768 | Q56VL3 | OCAD2 | OCIA domain-containing protein 2 |
| 2769 | Q5H9U9 | DDX6L | Probable ATP-dependent RNA helicase DDX60-like |
| 2770 | Q5JRX3 | PREP | Presequence protease, mitochondrial |
| 2771 | Q5SW79 | CE170 | Centrosomal protein of 170 kDa |
| 2772 | Q5T200 | ZC3HD | Zinc finger CCCH domain-containing protein 13 |
| 2773 | Q5T4S7 | UBR4 | E3 ubiquitin-protein ligase UBR4 |
| 2774 | Q5T7M9 | FA69A | Protein FAM69A |
| 2775 | Q5TDH0 | DDI2 | Protein DDI1 homolog 2 |
| 2776 | Q5VIR6 | VPS53 | Vacuolar protein sorting-associated protein 53 homolog |
| 2777 | Q5VT66 | 36951 | Mitochondrial amidoxime-reducing component 1 |
| 2778 | Q5VW32 | BROX | BRO1 domain-containing protein BROX |
| 2779 | Q6FHJ7 | SFRP4 | Secreted frizzled-related protein 4 |
| 2780 | Q6IBS0 | TWF2 | Twinfilin-2 |
| 2781 | Q6IQ49 | SDE2 | Protein SDE2 homolog |
| 2782 | Q6N021 | TET2 | Methylcytosine dioxygenase TET2 |
| 2783 | Q6NUM9 | RETST | All-trans-retinol 13,14-reductase |
| 2784 | Q6NY19 | KANK3 | KN motif and ankyrin repeat domain-containing protein 3 |
| 2785 | Q6NZY7 | BORG3 | Cdc42 effector protein 5 |
| 2786 | Q6P1J9 | CDC73 | Parafibromin |
| 2787 | Q6P2Q9 | PRP8 | Pre-mRNA-processing-splicing factor 8 |
| 2788 | Q6P2S7 | TTC41 | Tetratricopeptide repeat protein GNN |
| 2789 | Q6PD62 | CTR9 | RNA polymerase-associated protein CTR9 homolog |
| 2790 | Q6QAJ8 | TM220 | Transmembrane protein 220 |
| 2791 | Q6UWY5 | OLFL1 | Olfactomedin-like protein 1 |
| 2792 | Q6XQN6 | PNCB | Nicotinate phosphoribosyltransferase |
| 2793 | Q6ZMP0 | THSD4 | Thrombospondin type-1 domain-containing protein 4 |
| 2794 | Q6ZNG1 | ZN600 | Zinc finger protein 600 |
| 2795 | Q6ZRS4 | CC129 | Coiled-coil domain-containing protein 129 |
| 2796 | Q6ZUT6 | CO052 | Uncharacterized protein C15orf52 |
| 2797 | Q709C8 | VP13C | Vacuolar protein sorting-associated protein 13C |
| 2798 | Q7L014 | DDX46 | Probable ATP-dependent RNA helicase DDX46 |
| 2799 | Q7L0L9 | YA043 | Transmembrane protein LOC653160 |
| 2800 | Q7L576 | CYFP1 | Cytoplasmic FMR1-interacting protein 1 |
| 2801 | Q7Z5K2 | WAPL | Wings apart-like protein homolog |
| 2802 | Q7Z5L7 | PODN | Podocan |
| 2803 | Q7Z7H5 | TMED4 | Transmembrane emp24 domain-containing protein 4 |
| 2804 | Q86UX7 | URP2 | Fermitin family homolog 3 |
| 2805 | Q86VP6 | CAND1 | Cullin-associated NEDD8-dissociated protein 1 |
| 2806 | Q86VS8 | HOOK3 | Protein Hook homolog 3 |
| 2807 | Q86WA6 | BPHL | Valacyclovir hydrolase |
| 2808 | Q86WU2 | LDHD | Probable D-lactate dehydrogenase, mitochondrial |
| 2809 | Q86Z14 | KLOTB | Beta-klotho |
| 2810 | Q8IUX7 | AEBP1 | Adipocyte enhancer-binding protein 1 |
| 2811 | Q8IVL6 | P3H3 | Prolyl 3-hydroxylase 3 |
| 2812 | Q8IVT2 | MISP | Mitotic interactor and substrate of PLK1 |
| 2813 | Q8IW45 | NNRD | ATP-dependent (S)-NAD(P)H-hydrate dehydratase |
| 2814 | Q8IXL7 | MSRB3 | Methionine-R-sulfoxide reductase B3 |
| 2815 | Q8IXS6 | PALM2 | Paralemmin-2 |
| 2816 | Q8IZ73 | RUSD2 | RNA pseudouridylate synthase domain-containing protein 2 |
| 2817 | Q8IZ83 | A16A1 | Aldehyde dehydrogenase family 16 member A1 |
| 2818 | Q8N0X7 | SPG20 | Spartin |
| 2819 | Q8N129 | CNPY4 | Protein canopy homolog 4 |
| 2820 | Q8N3D4 | EH1L1 | EH domain-binding protein 1-like protein 1 |
| 2821 | Q8N4C6 | NIN | Ninein |
| 2822 | Q8N5A5 | ZGPAT | Zinc finger CCCH-type with G patch domain-containing protein |
| 2823 | Q8NBJ5 | GT251 | Procollagen galactosyltransferase 1 |
| 2824 | Q8NG27 | PJA1 | E3 ubiquitin-protein ligase Praja-1 |
| 2825 | Q8TD55 | PKHO2 | Pleckstrin homology domain-containing family O member 2 |
| 2826 | Q8TDB6 | DTX3L | E3 ubiquitin-protein ligase DTX3L |
| 2827 | Q8WTS6 | SETD7 | Histone-lysine N-methyltransferase SETD7 |
| 2828 | Q8WU39 | MZB1 | Marginal zone B- and B1-cell-specific protein |
| 2829 | Q8WUR7 | CO040 | UPF0235 protein C15orf40 |
| 2830 | Q8VW28 | BLNK | B-cell linker protein |
| 2831 | Q8WW9 | HNRLL | Heterogeneous nuclear ribonucleoprotein L-like |
| 2832 | Q8WWM9 | CYGB | Cytoglobin |
| 2833 | Q8WYQ3 | CHC10 | Coiled-coil-helix-coiled-coil-helix domain-containing protein 10, |
| | | | mitochondrial |
| 2834 | Q92504 | S39A7 | Zinc transporter SLC39A7 |
| 2835 | Q92530 | PSMF1 | Proteasome inhibitor PI31 subunit |
| 2836 | Q92743 | HTRA1 | Serine protease HTRA1 |
| 2837 | Q92817 | EVPL | Envoplakin |
| 2838 | Q92839 | HYAS1 | Hyaluronan synthase 1 |
| 2839 | Q92844 | TANK | TRAF family member-associated NF-kappa-B activator |
| 2840 | Q92882 | OSTF1 | Osteoclast-stimulating factor 1 |
| 2841 | Q92890 | UFD1 | Ubiquitin fusion degradation protein 1 homolog |
| 2842 | Q92954 | PRG4 | Proteoglycan 4; Proteoglycan 4 C-terminal part |
| 2843 | Q93091 | RNAS6 | Ribonuclease K6 |
| 2844 | Q96A73 | P33MX | Putative monooxygenase p33MONOX |
| 2845 | Q96A84 | EMID1 | EMI domain-containing protein 1 |
| 2846 | Q96AC1 | FERM2 | Fermitin family homolog 2 |
| 2847 | Q96AY3 | FKB10 | Peptidyl-prolyl cis-trans isomerase FKBP10 |
| 2848 | Q96CG8 | CTHR1 | Collagen triple helix repeat-containing protein 1 |
| 2849 | Q96CW1 | AP2M1 | AP-2 complex subunit mu |
| 2850 | Q96DB5 | RMD1 | Regulator of microtubule dynamics protein 1 |
| 2851 | Q96F85 | CNRP1 | CB1 cannabinoid receptor-interacting protein 1 |
| 2852 | Q96FQ6 | S10AG | Protein S100-A16 |
| 2853 | Q96FS4 | SIPA1 | Signal-induced proliferation-associated protein 1 |
| 2854 | Q96HA7 | TONSL | Tonsoku-like protein |
| 2855 | Q96IX5 | USMG5 | Up-regulated during skeletal muscle growth protein 5 |
| 2856 | Q96K76 | UBP47 | Ubiquitin carboxyl-terminal hydrolase 47 |
| 2857 | Q96PU8 | QKI | Protein quaking |
| 2858 | Q96PZ0 | PUS7 | Pseudouridylate synthase 7 homolog |
| 2859 | Q96RU3 | FNBP1 | Formin-binding protein 1 |
| 2860 | Q96SM3 | CPXM1 | Probable carboxypeptidase X1 |
| 2861 | Q99613 | EIF3C | Eukaryotic translation initiation factor 3 subunit C |
| 2862 | Q99653 | CHP1 | Calcineurin B homologous protein 1 |
| 2863 | Q99759 | M3K3 | Mitogen-activated protein kinase kinase kinase 3 |
| 2864 | Q99873 | ANM1 | Protein arginine N-methyltransferase 1 |
| 2865 | Q99952 | PTN18 | Tyrosine-protein phosphatase non-receptor type 18 |
| 2866 | Q99983 | OMD | Osteomodulin |
| 2867 | Q9BQ61 | CS043 | Uncharacterized protein C19orf43 |
| 2868 | Q9BQB6 | VKOR1 | Vitamin K epoxide reductase complex subunit 1 |
| 2869 | Q9BRF8 | CPPED | Serine/threonine-protein phosphatase CPPED1 |
| 2870 | Q9BRR6 | ADPGK | ADP-dependent glucokinase |
| 2871 | Q9BRX8 | F213A | Redox-regulatory protein FAM213A |
| 2872 | Q9BSJ8 | ESYT1 | Extended synaptotagmin-1 |
| 2873 | Q9BT23 | LIMD2 | LIM domain-containing protein 2 |
| 2874 | Q9BTP6 | ZBED2 | Zinc finger BED domain-containing protein 2 |
| 2875 | Q9BTV4 | TMM43 | Transmembrane protein 43 |
| 2876 | Q9BUF5 | TBB6 | Tubulin beta-6 chain |
| 2877 | Q9BUP0 | EFHD1 | EF-hand domain-containing protein D1 |
| 2878 | Q9BV57 | MTND | 1,2-dihydroxy-3-keto-5-methylthiopentene dioxygenase |
| 2879 | Q9BVA1 | TBB2B | Tubulin beta-2B chain |
| 2880 | Q9BWM7 | SFXN3 | Sideroflexin-3 |
| 2881 | Q9BXJ4 | C1QT3 | Complement C1q tumor necrosis factor-related protein 3 |
| 2882 | Q9BXX0 | EMIL2 | EMILIN-2 |
| 2883 | Q9BZF2 | OSBL7 | Oxysterol-binding protein-related protein 7 |
| 2884 | Q9BZZ2 | SN | Sialoadhesin |
| 2885 | Q9C075 | K1C23 | Keratin, type I cytoskeletal 23 |
| 2886 | Q9C086 | IN80B | INO80 complex subunit B |
| 2887 | Q9GIY3 | 2B1E | HLA class II histocompatibility antigen, DRB1-14 beta chain |
| 2888 | Q9GZP8 | IMUP | Immortalization up-regulated protein |
| 2889 | Q9GZS3 | WDR61 | WD repeat-containing protein 61; WD repeat-containing protein 61, N- |
| | | | terminally processed |
| 2890 | Q9GZU8 | F192A | Protein FAM192A |
| 2891 | Q9H061 | T126A | Transmembrane protein 126A |
| 2892 | Q9H0L4 | CSTFT | Cleavage stimulation factor subunit 2 tau variant |
| 2893 | Q9H1A4 | APC1 | Anaphase-promoting complex subunit 1 |
| 2894 | Q9H3N1 | TMX1 | Thioredoxin-related transmembrane protein 1 |
| 2895 | Q9H4G4 | GAPR1 | Golgi-associated plant pathogenesis-related protein 1 |
| 2896 | Q9H4M9 | EHD1 | EH domain-containing protein 1 |
| 2897 | Q9H9B4 | SFXN1 | Sideroflexin-1 |
| 2898 | Q9HAK2 | COE2 | Transcription factor COE2 |
| 2899 | Q9HB96 | FANCE | Fanconi anemia group E protein |
| 2900 | Q9HCU0 | CD248 | Endosialin |
| 2901 | Q9HCY8 | S10AE | Protein S100-A14 |
| 2902 | Q9HD89 | RETN | Resistin |
| 2903 | Q9NQ48 | LZTL1 | Leucine zipper transcription factor-like protein 1 |
| 2904 | Q9NQP4 | PFD4 | Prefoldin subunit 4 |
| 2905 | Q9NR19 | ACSA | Acetyl-coenzyme A synthetase, cytoplasmic |
| 2906 | Q9NR99 | MXRA5 | Matrix-remodeling-associated protein 5 |
| 2907 | Q9NRM1 | ENAM | Enamelin |
| 2908 | Q9NRN5 | OLFL3 | Olfactomedin-like protein 3 |
| 2909 | Q9NS15 | LTBP3 | Latent-transforming growth factor beta-binding protein 3 |
| 2910 | Q9NS62 | THSD1 | Thrombospondin type-1 domain-containing protein 1 |
| 2911 | Q9NT62 | ATG3 | Ubiquitin-like-conjugating enzyme ATG3 |
| 2912 | Q9NUQ9 | FA49B | Protein FAM49B |
| 2913 | Q9NVD7 | PARVA | Alpha-parvin |
| 2914 | Q9NVJ2 | ARL8B | ADP-ribosylation factor-like protein 8B |
| 2915 | Q9NY15 | STAB1 | Stabilin-1 |
| 2916 | Q9NYV4 | CDK12 | Cyclin-dependent kinase 12 |
| 2917 | Q9NZ08 | ERAP1 | Endoplasmic reticulum aminopeptidase 1 |
| 2918 | Q9NZD4 | AHSP | Alpha-hemoglobin-stabilizing protein |
| 2919 | Q9NZM1 | MYOF | Myoferlin |
| 2920 | Q9P0K7 | RAI14 | Ankycorbin |
| 2921 | Q9P0M6 | H2AW | Core histone macro-H2A.2 |
| 2922 | Q9P2A4 | ABI3 | ABI gene family member 3 |
| 2923 | Q9UBF2 | COPG2 | Coatomer subunit gamma-2 |
| 2924 | Q9UBQ0 | VPS29 | Vacuolar protein sorting-associated protein 29 |
| 2925 | Q9UBR2 | CATZ | Cathepsin Z |
| 2926 | Q9UBW5 | BIN2 | Bridging integrator 2 |
| 2927 | Q9UFN0 | NPS3A | Protein NipSnap homolog 3A |
| 2928 | Q9UGI9 | AAKG3 | 5′-AMP-activated protein kinase subunit gamma-3 |
| 2929 | Q9UHL4 | DPP2 | Dipeptidyl peptidase 2 |
| 2930 | Q9UHQ9 | NB5R1 | NADH-cytochrome b5 reductase 1 |
| 2931 | Q9UHR4 | BI2L1 | Brain-specific angiogenesis inhibitor 1-associated protein 2-like protein 1 |
| 2932 | Q9UI08 | EVL | Ena/VASP-like protein |
| 2933 | Q9UKK3 | PARP4 | Poly [ADP-ribose] polymerase 4 |
| 2934 | Q9UKU9 | ANGL2 | Angiopoietin-related protein 2 |
| 2935 | Q9UL25 | RAB21 | Ras-related protein Rab-21 |
| 2936 | Q9UMD9 | COHA1 | 120 kDa linear IgA disease antigen; 97 kDa linear IgA disease |
| | | | antigen; Collagen alpha-1(XVII) chain |
| 2937 | Q9UNM6 | PSD13 | 26S proteasome non-ATPase regulatory subunit 13 |
| 2938 | Q9UPN3 | MACF1 | Microtubule-actin cross-linking factor 1, isoforms 1/2/3/5 |
| 2939 | Q9UPW6 | SATB2 | DNA-binding protein SATB2 |
| 2940 | Q9Y2B9 | IPKG | cAMP-dependent protein kinase inhibitor gamma |
| 2941 | Q9Y2H5 | PKHA6 | Pleckstrin homology domain-containing family A member 6 |
| 2942 | Q9Y2Y8 | PRG3 | Proteoglycan 3 |
| 2943 | Q9Y316 | MEMO1 | Protein MEMO1 |
| 2944 | Q9Y371 | SHLB1 | Endophilin-B1 |
| 2945 | Q9Y4G6 | TLN2 | Talin-2 |
| 2946 | Q9Y4H4 | GPSM3 | G-protein-signaling modulator 3 |
| 2947 | Q9Y5P4 | C43BP | Collagen type IV alpha-3-binding protein |
| 2948 | Q9Y5X3 | SNX5 | Sorting nexin-5 |
| 2949 | Q9Y6A5 | TACC3 | Transforming acidic coiled-coil-containing protein 3 |
| 2950 | Q9Y6M9 | NDUB9 | NADH dehydrogenase [ubiquinone] 1 beta subcomplex subunit 9 |

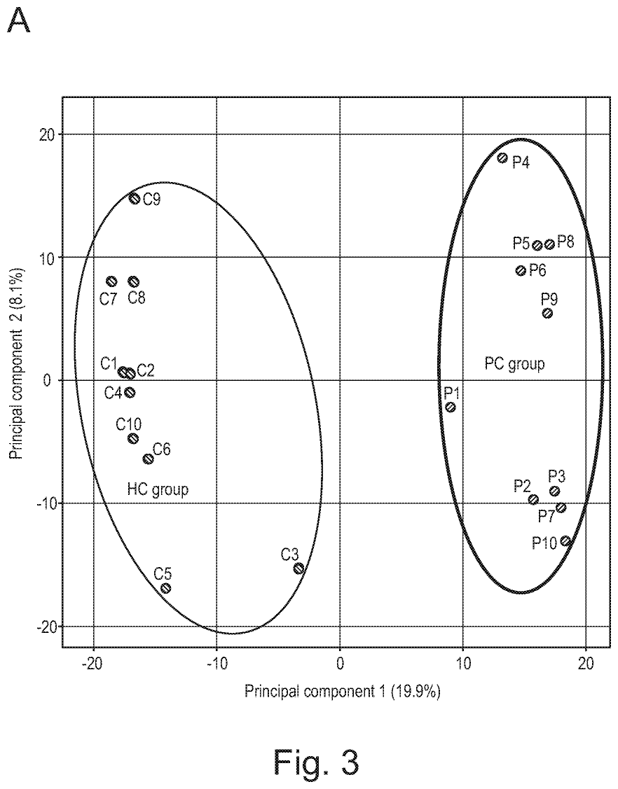

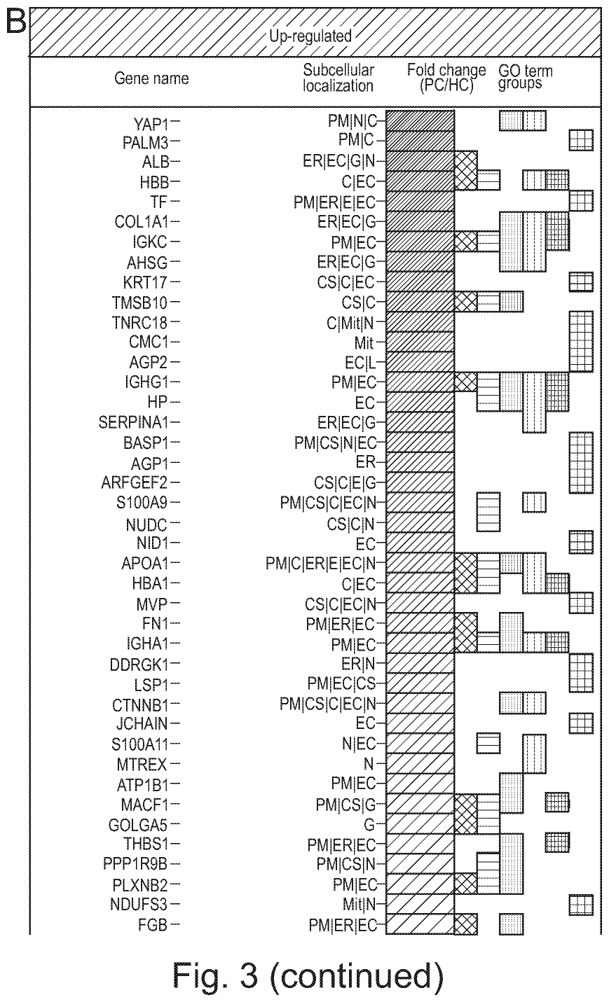

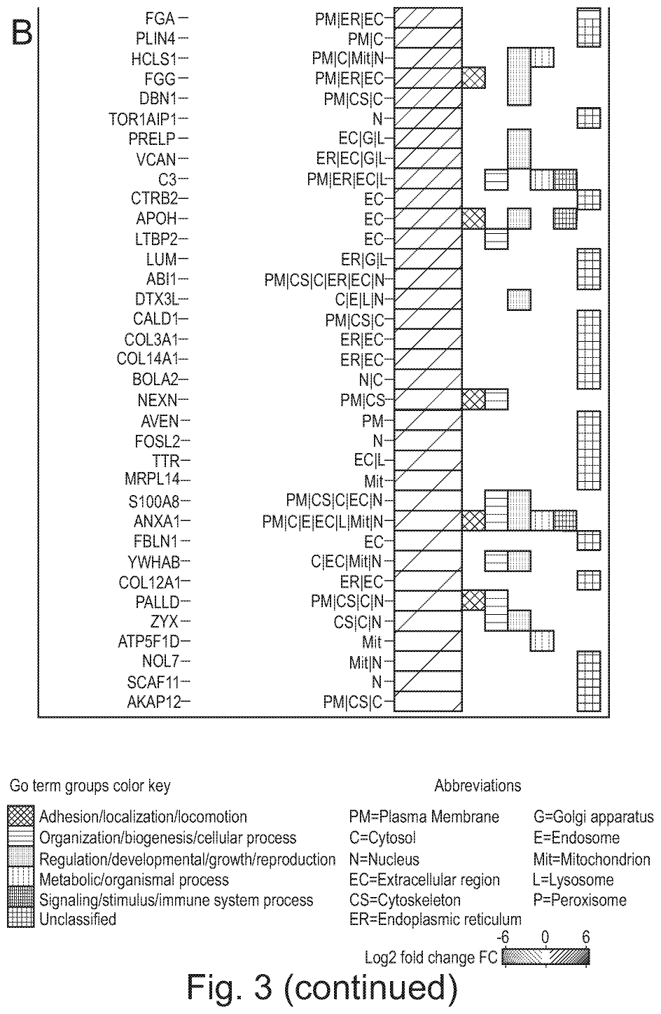

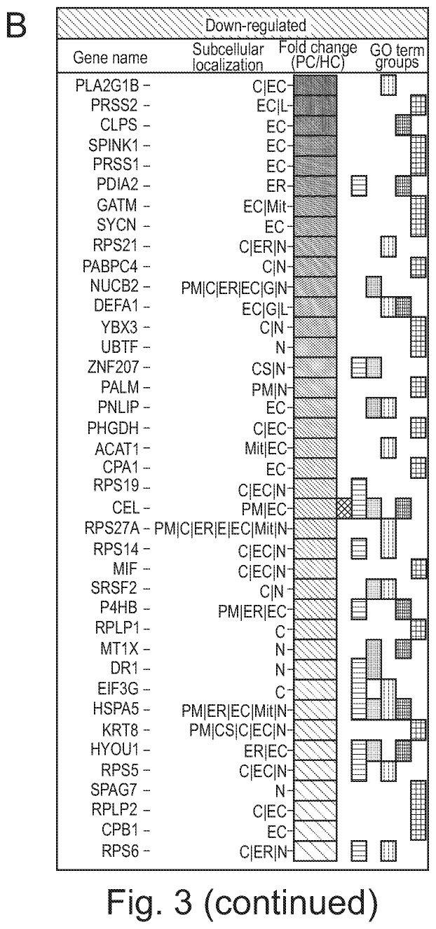

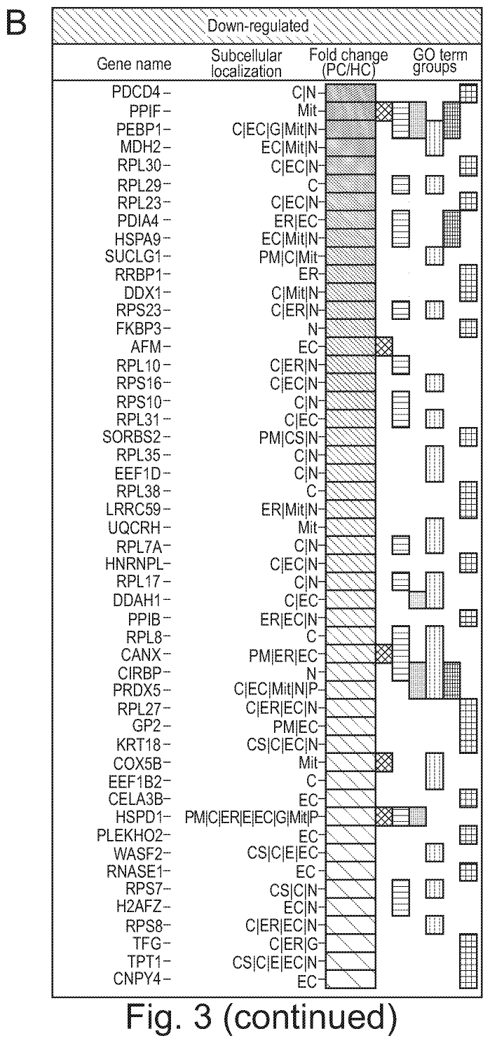

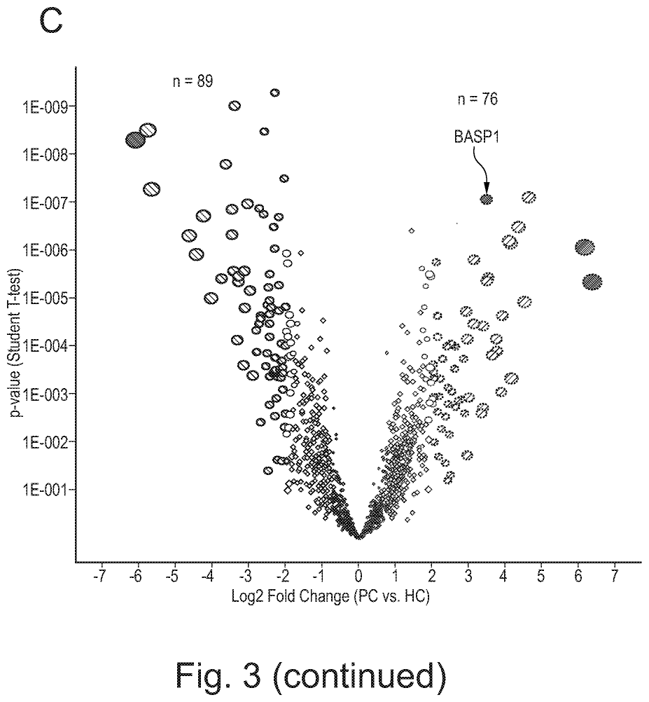

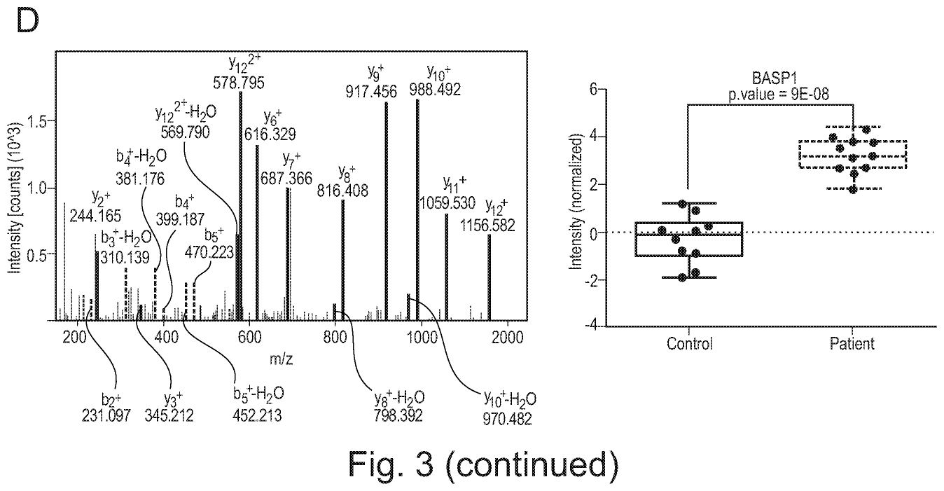

Among the quantified proteins, 2264 proteins were present in the pancreatic cancer group and 2354 proteins in the healthy control group, respectively. To demonstrate the general pattern of protein abundance variation within and between different groups, a two-dimensional Principal Component Analysis (PCA) was performed based on all quantified proteins by an online tool ClustVis (Metsalu T, et al. Nucleic Acids Res 2015; 43(W1):W566-70). Using the log 2-ratio of each sample over the mean of all samples, a complete separation of the pancreatic cancer and healthy control groups was observed (FIG. 3, (A)).

By employing the criteria of FDR adjusted p-value (or q-value) of 0.01, S0=2, the number of peptides N1 and the fold change N2 as a cut-off, a total of 165 proteins with two or more unique peptides were significantly differentially expressed between the two experimental groups (FIG. 3 (B)). A volcano plot of significantly upregulated and down-regulated proteins is presented in FIG. 3 (C).

### Development of Targeted Protein Assays Using PRM

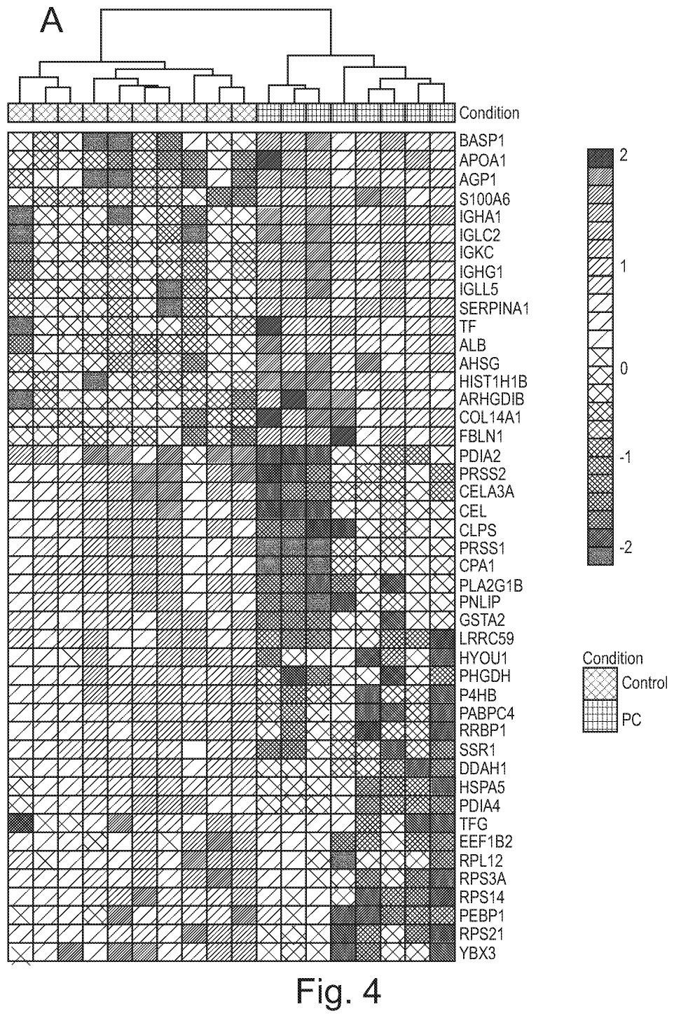

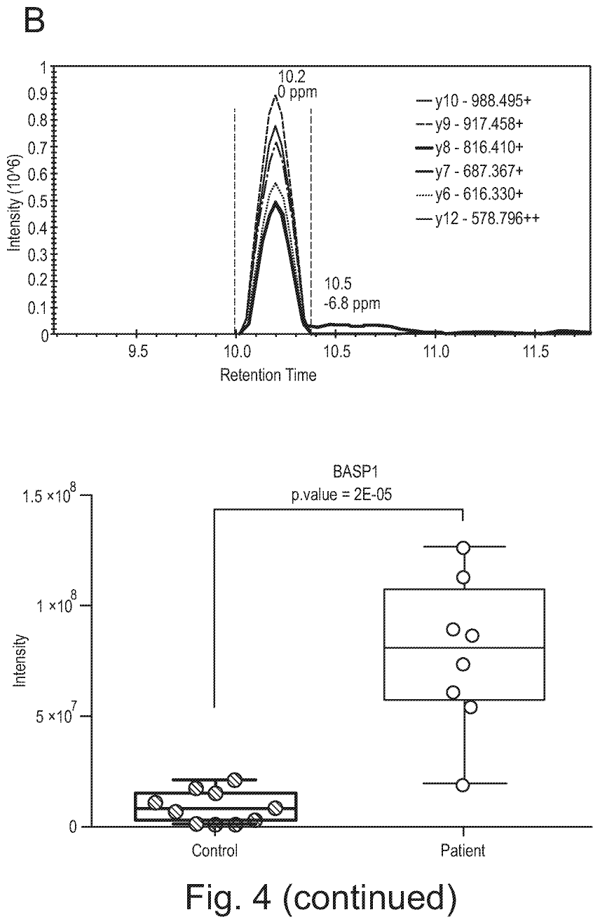

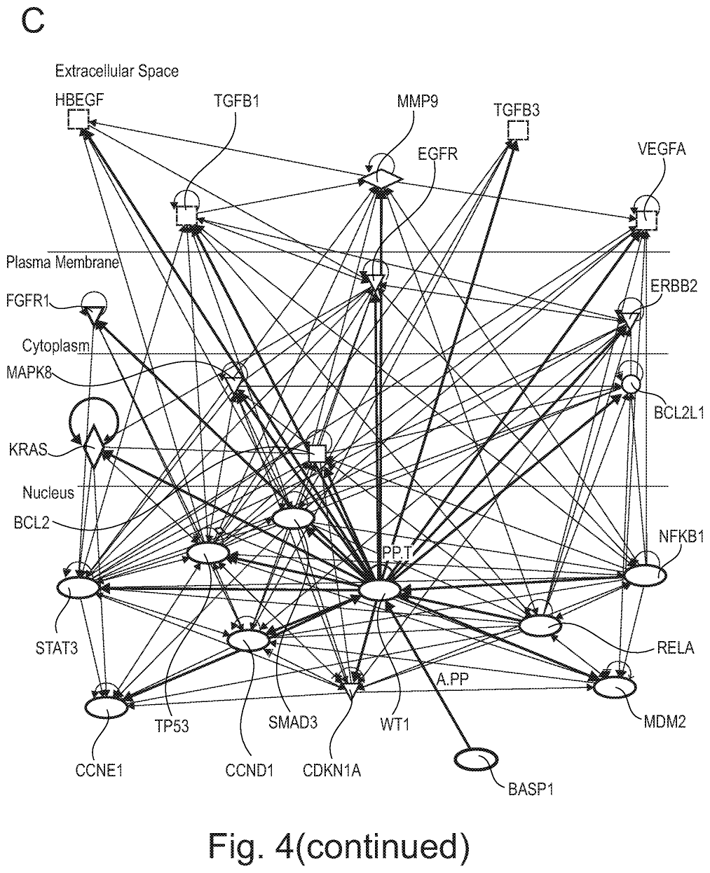

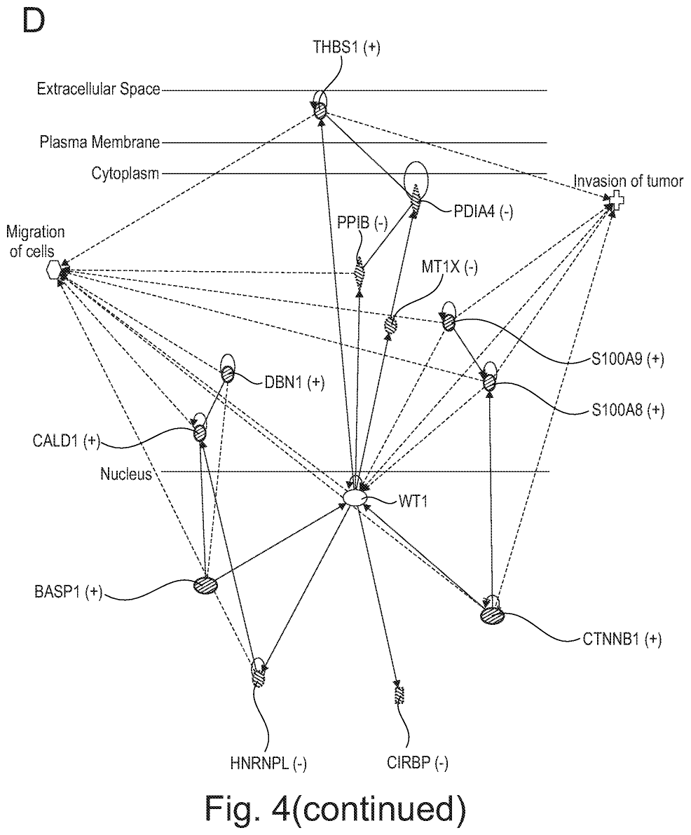

To verify the differential expression changes of potential protein biomarkers from MS discovery, PRM was employed based on the same samples from the MS discovery phase (n=8 in the pancreatic cancer group and n=10 in the healthy control group). Eighty-one proteins with one or two unique peptides for each protein were selected and a panel of 45 proteins were successfully detected and quantified. Among these proteins, 17 proteins were significantly up-regulated (p<01), while 28 proteins were down-regulated in pancreatic cancer versus healthy controls, respectively (Tables 1 and 2, below). From the panel of 45 verified candidates, 16 extracellular proteins emerged that could theoretically be detected in serum and potentially be applied in noninvasive diagnosis and/or prognosis prediction, including S100A6, TF, FBLN1, HYOU1, PNLIP, P4HB, AHSG, PLA2G1B, AGP1, PRSS1, PRSS2, APOA1, ALB, SERPINAL CLPS, and COL14A1 as previously reported by our group (Zhou Q, et al. Alpha-1-acid glycoprotein 1 is a diagnostic and prognostic biomarker for pancreatic cancer; 2019). Subsequently, a consensus clustering heatmap was created based on the 45 verified proteins and a clear discrimination between pancreatic cancer and healthy controls was observed (FIG. 4 (A)).

### Selection of BASP1 for Further Validation

BASP1 is a neuron enriched Ca(2+)-dependent calmodulin-binding protein with unknown function in pancreatic cancer. BASP1 was established as a top-ranked protein, being significantly up-regulated in the pancreatic cancer group by a fold change of 11.24, p=9E-08 (FIG. 3 (D)). Notably, based on quantification of the following unique peptides: SDGAPASDSKPGSSEAAPSSK (SEQ ID NO: 46) and ETPAATEAPSSTPK (SEQ ID NO: 12), BASP1 presented as one of the most reproducible candidates, being significantly up-regulated in the pancreatic cancer group with a fold change of 12.91 and p=2E-05 (FIG. 4 (B)). As a potential novel biomarker, BASP1 was selected for further validation by bioinformatic and clinical association studies.

### BASP1 is Functionally Related to WT

In order to obtain an unbiased overview of the BASP1 functional relationships in a biological context, Ingenuity Pathway Analysis (IPA) was used to create a network involving all proteins with direct relationships (e.g. physical interaction or direct activation) to BASP1. This analysis, building upon a literature-derived relationship knowledge base, yielded a network including 412 proteins that were significantly enriched and involved in several canonical pathways (e.g., pancreatic adenocarcinoma signaling, regulation of the epithelial-mesenchymal transition pathway, ILK signaling, as well as tumorigenic conditions (e.g. apoptosis, cell migration, angiogenesis). Furthermore, among the top upstream regulators automatically identified by the IPA algorithm for the BASP1 interactor set, several well known tumor-related signaling proteins emerged (e.g., TP53, TNF, TGFB1, EGF, HRAS).

The pathway analysis may suggest that the link between BASP1 and pancreatic cancer is via WT1, and there are 21 proteins from the pancreatic adenocarcinoma signaling pathway that interact with WT1 (enrichment p-value 3E-16, FIG. 4 (C)). Among these, extracellular signaling molecules TGFB1, TGFB3, VEGFA, HBEGF, receptor tyrosine kinases EGFR1, ERBB2 and FGFR1, apoptosis regulators BCL2, BCL2L1 and the recognized pancreatic cancer-related transcription regulator TP53, KRAS, and MAPK8 were annotated. Mapping of the differentially expressed proteins into the BASP1/WT1 network provided 11 hits out of 165 (FIG. 4 (D)). Markedly, according to IPA analysis, most of these proteins are involved in cellular migration and tumor invasion processes.

### BASP1 and WT1 Expression in Tumor Samples and Cancer Cell Line

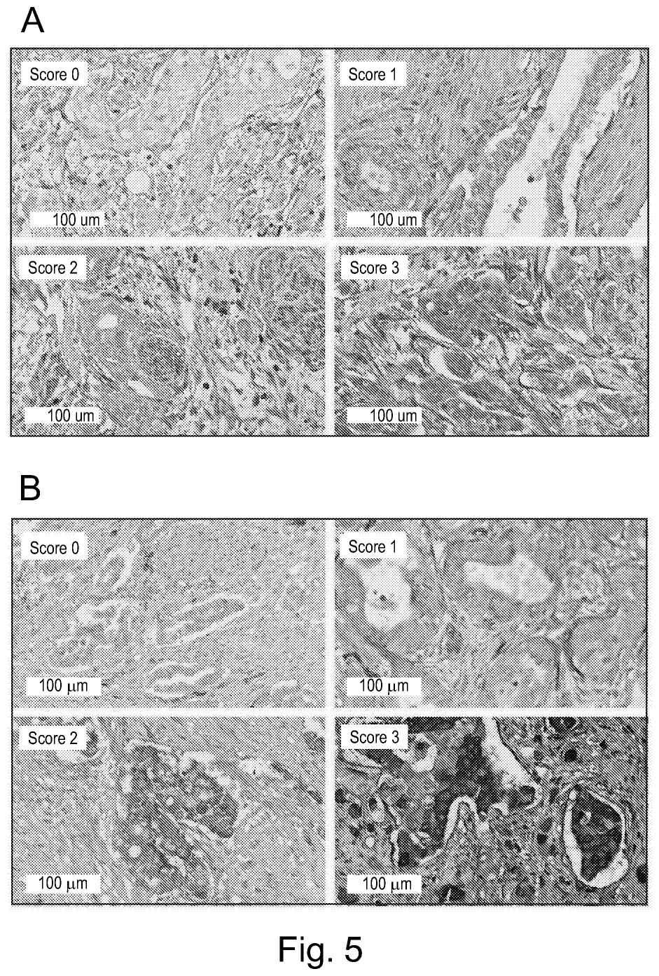

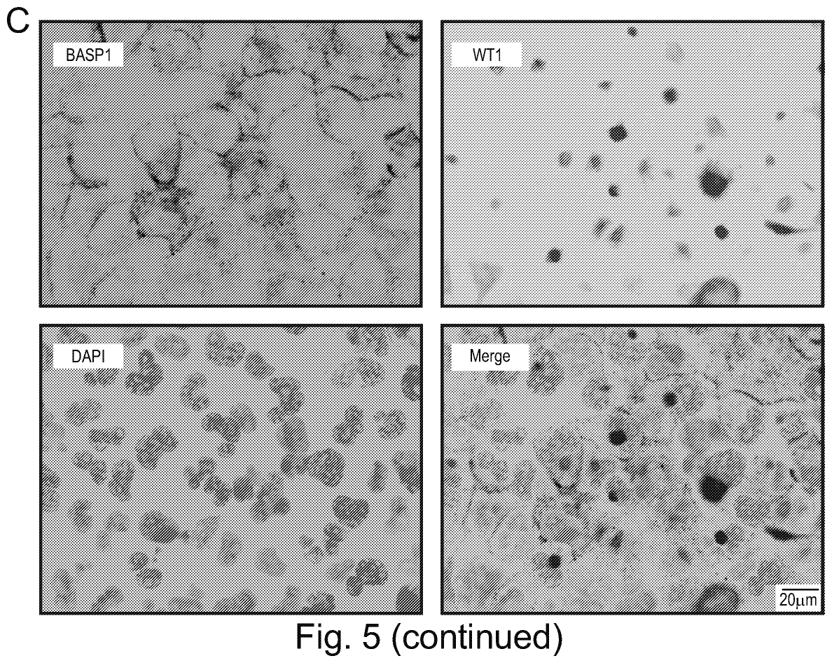

The expression levels of BASP1 and WT1 were assessed in a larger cohort of pancreatic cancer patients by TMA-IHC. The clinical characteristics of the pancreatic cancer patients are shown in Table 3, below. Based on the validation cohort, 141 patients were successfully scored for BASP1 and 139 patients for WT1, respectively. Both markers were evaluable in 137 patients. In the BASP1 cohort (n=141), 15 (10.6%) tissue samples from pancreatic cancer patients showed negative staining (Score 0) and 126 (89.4%) samples displayed positive staining, where 25 (17.7%) samples were scored as weak (Score 1), 66 (46.8%) as moderate (Score 2), and 35 (24.8%) as strong (Score 3). The majority of the staining was observed accentuated in the cytoplasm/plasma membrane (PM), accompanied by weak nuclear staining (FIG. 5 (A)). 135 (97.1%) pancreatic cancer tissue samples had positive staining of WT1 protein in the WT1 cohort (n=139), and only 4 (2.9%) were observed as loss of positivity (Score 0). Moreover, the WT1 staining was predominantly presented in the cytoplasm of pancreatic tumor cells, while nuclear immunostaining was weak. Furthermore, the positively stained tissue samples were subdivided into weak 22 (15.8%, Score 1), moderate 51 (36.7%, Score 2), and strong 62 (44.6%, Score 3) staining (FIG. 5 (B)).

In order to study the dual expression patterns of BASP1 and WT1 in human pancreatic cancer cell line, immunofluorescence staining of BASP1 and WT1 in PANC-1 cell line was also performed. In accordance with the IHC results, BASP1 was mostly expressed in cytoplasm and PM, while WT1 was detected in the cytoplasm and mostly with perinuclear localization (FIG. 5 (C)).

### BASP1 Expression is an Independent Predictor of Favorable Survival

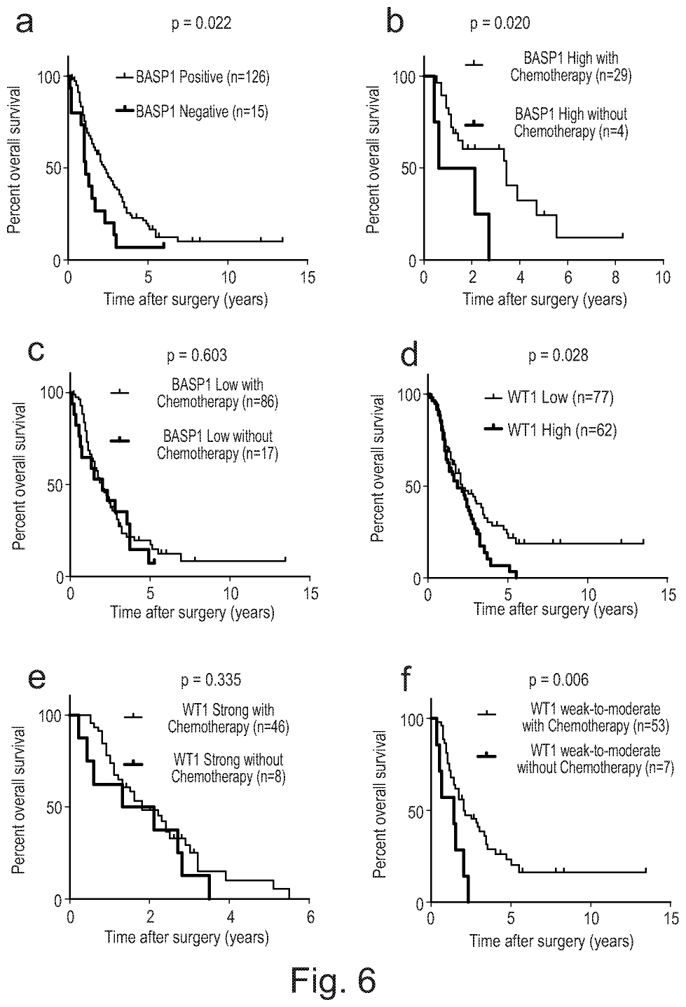

Kaplan-Meier analysis showed that pancreatic cancer patients with positive BASP1 expression had significantly prolonged overall survival (OS) compared to patients with negative BASP1 expression (median survival, 27.7 vs. 13.3 months, respectively, p=0.022, FIG. 6 (A)). The univariable Cox regression analysis indicated that apart from BASP1 positive expression (p=0.025), three other variables, including smoking history (p=0.015), presenting symptoms at diagnosis (p=0.044), and histological grade (p=0.041), were correlated with OS. In multivariable Cox regression analysis, positive BASP1 expression remained an independent prognostic factor with a hazard ratio (HR) of 0.468, 95% confidence interval (CI) 0.257-0.852, and p=0.013 (Table 4, below).

### High BASP1 Expression Predicts Beneficial Response to Adjuvant Chemotherapy

In the BASP1 cohort, patients with high expression of BASP1 (Score 3) exhibited significantly improved OS when they received adjuvant chemotherapy compared to those without adjuvant chemotherapy (median survival, 40.5 vs. 7.2 months, respectively, p=0.020, FIG. 6 (B)). No correlation to adjuvant chemotherapy (p=0.603) was observed in patients with low expression of BASP1 (score 0, 1, and 2, FIG. 6 (C)). These results suggest that BASP1 may function both as a marker for favorable prognosis and as a predictive biomarker for positive adjuvant chemotherapy response.

### WT1 Expression is Correlated to Poor Survival and Chemoresistance

Kaplan-Meier analysis revealed that patients in the high WT1 expression (Score 3) group had significantly shorter OS compared to those in the low WT1 expression (Score 0, 1, and 2) group (median survival, 22.2 vs. 25.7 months, respectively, p=0.028, FIG. 6 (D)). Further univariable Cox regression analysis demonstrated that besides high WT1 expression (p=0.029), other factors such as smoking history (p=0.012), presenting symptoms at diagnosis (p=0.049), and high pathological grades (p=0.035) were also associated with OS. In multivariable Cox regression analysis, high WT1 expression was identified as an independent factor associated with OS (HR 1.636, 95% CI 1.083-2.473, p=0.019, Table 4).

In pancreatic cancer patients with strong expression of WT1, adjuvant chemotherapy displayed no significant impact on OS (p=0.335, FIG. 6 (E)). Of note, pancreatic cancer patients with weak-to moderate WT1 expression, who received adjuvant chemotherapy presented significantly extended OS compared to patients that did not receive chemotherapy (median survival, 24.5 vs. 16.9 months, respectively, p=0.006, FIG. 6 (F)). These findings indicate that WT1 expression may be correlated with chemoresistance in pancreatic cancer.

Patients with Negative BASP1 and High WT1 Expression have the Poorest Outcome

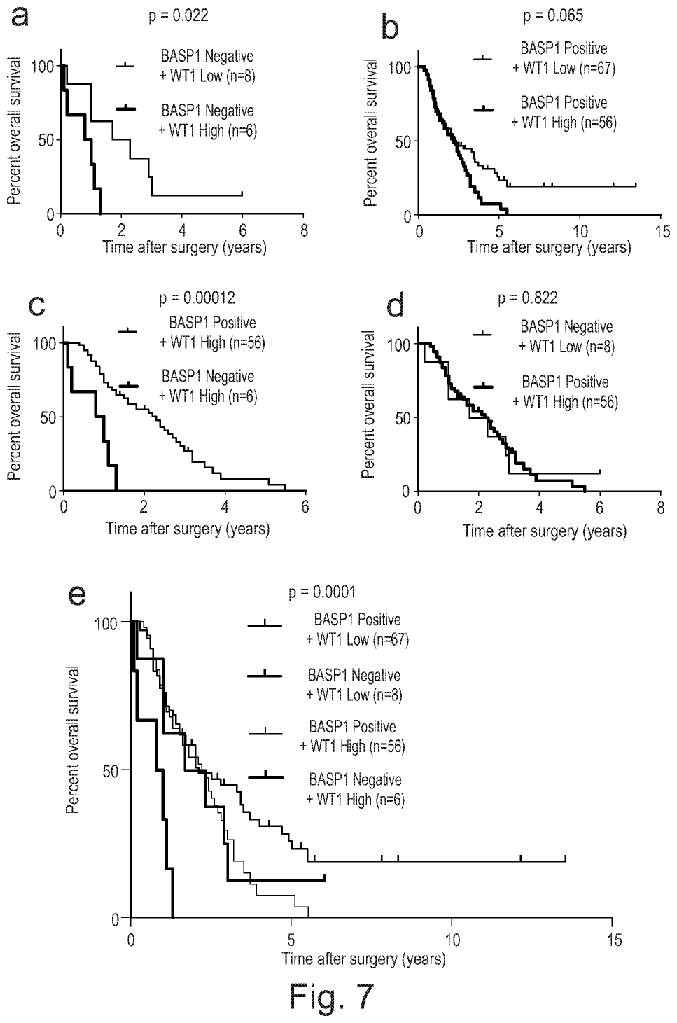

To examine the potential biological cross-talk between BASP1 and WT1 in terms of patient survival, subgroup functionality analysis of these prognostic markers was performed. For patients with negative expression of BASP1, the high WT1 expression group had significantly reduced OS compared to the low WT1 expression group (median survival, 9.4 vs. 20.4 months, respectively, p=0.022, FIG. 7 (A)). No significant difference in OS between high and low WT1 groups was observed in patients with positive expression of BASP1 (p=0.065, FIG. 7 (B)). These data suggested that BASP1 can potentially relieve the oncogenic effect of WT1 in pancreatic cancer patients.

Moreover, for patients with high WT1 expression, the positive BASP1 expression group presented significantly prolonged OS compared to the BASP1 negative group (median survival, 25.8 vs. 9.4 months, respectively, p=0.00012, FIG. 7 (C)). In addition, patients with high WT1 and positive BASP1 expression presented a similar survival pattern as the group of patients with low WT1 and negative BASP1 expression (p=0.822, FIG. 7 (D)). The results confirm the possibility that the protective role of BASP1 can impede the tumor promoting function of WT1. Finally, the best prognosis was seen in patients with positive expression of BASP1 and low expression of WT1, whereas patients with negative BASP1 expression and high WT1 expression had the poorest outcome (median survival, 25.7 vs. 9.4 months, p=0.0001, FIG. 7 (E)). The multivariable Cox regression analysis, highlighted negative BASP1 expression and high WT1 expression as an independent factor associated with significantly shortened OS (HR 3.536, 95% CI 1.336-9.362, p=0.011). These data suggest that BASP1 may act as a tumor suppressor rescuing the oncogenic effect of overexpressed WT1.

TABLE 1
PRM verified upregulated proteins in pancreatic cancer compared to healthy controls (ranked according to fold change).

| Sr. | UniProt | | | | SEQ | | Fold change |
|---|---|---|---|---|---|---|---|
| no. | accession | Gene | Protein name | Unique peptide | ID NO | P value | (PC/NC) |
| 1 | P02647 | APOA1 | Apolipoprotein A-I | K.LLDNWDSVTSTFSK.L | 1 | 1.8E−08 | 39.12 |
| 2 | B9A064 | IGLL5 | Immunoglobulin lambda-like polypeptide 5 | K.VTVLGQPK.A | 2 | 3.5E−09 | 35.02 |
| 3 | P02765 | AHSG | Alpha-2-HS-glycoprotein | K.FSVVYAK•C | 3 | 2.1E−09 | 27.47 |
| 4 | P0DOY2 | IGLC2 | Immunoglobulin lambda constant 2 | K.AAPSVTLFPPSSEELQANK.A | 4 | 1.6E−09 | 24.42 |
| 5 | P02763 | AGP1 | Alpha-1-acid glycoprotein 1 | R.YVGGQEHFAHLLILR.D | 5 | 4.6E−06 | 24.25 |
| 6 | P01857 | IGHG1 | Immunoglobulin heavy constant gamma 1 | K.GPSVFPLAPSSK•S | 6 | 2.2E−10 | 23.59 |
| 7 | P01834 | IGKC | Immunoglobulin kappa constant | K.VDNALQSGNSQESVTEQDSK.D | 7 | 5.1E−11 | 22.78 |
| 8 | P01876 | IGHA1 | Immunoglobulin heavy constant alpha 1 | K.TPLTATLSK•S | 8 | 1.3E−09 | 20.82 |
| 9 | P02787 | TF | Serotransferrin | K.EGYYGYTGAFR.C | 9 | 1.6E−10 | 19.84 |
| 10 | P02768 | ALB | Serum albumin | K.DDNPNLPR.L | 10 | 4.5E−09 | 19.70 |
| 11 | P01009 | SERPINA1 | Alpha-1-antitrypsin | K.AVLTIDEK.G | 11 | 6.2E−10 | 17.03 |
| 12 | P80723 | BASP1 | Brain acid soluble protein 1 | K.ETPAATEAPSSTPK.A | 12 | 1.7E−05 | 12.91 |
| 13 | P06703 | S100A6 | Protein S100-A6 | K.LQDAEIAR.L | 13 | 1.5E−05 | 12.13 |
| 14 | Q05707 | COL14A1 | Collagen alpha-1(XIV) chain | R.YTAILNQIPSHSSSIR.T | 14 | 6.5E−12 | 10.70 |
| 15 | P16401 | HIST1H1B | Histone H1.5 | K.ATGPPVSELITK.A | 15 | 1.6E−08 | 9.85 |
| 16 | P23142 | FBLN1 | Fibulin-1 | K.IIEVEEEQEDPYLNDR.C | 16 | 4.8E−08 | 8.34 |
| 17 | P52566 | ARHGDIB | Rho GDP-dissociation inhibitor 2 | K.TLLGDGPVVTDPK.A | 17 | 1.9E−08 | 5.46 |

Abbreviations: NC, normal controls; PC, pancreatic cancer; PRM, parallel reaction monitoring.

TABLE 2
PRM verified downregulated proteins in pancreatic cancer compared to healthy controls (ranked according to fold change).

| Sr. | UniProt | | | | Seq | | Fold change |
|---|---|---|---|---|---|---|---|
| no. | accession | Gene | Protein name | Unique peptide | ID No. | P value | (NC/PC) |
| 1 | P04054 | PLA2G1B | Phospholipase A2 | R.AVWQFR.K | 18 | 1.1E−06 | 56.89 |
| 2 | P16233 | PNLIP | Pancreatic triacylglycerol lipase | R.TGYTQASQNIR.I | 19 | 8.7E−06 | 51.98 |
| 3 | P09093 | CELA3A | Chymotrypsin-like elastase family member 3A | R.WNWWGSTVK•K | 20 | 1.7E−06 | 47.18 |
| 4 | P04118 | CLPS | Colipase | K.TLYGIYYK•C | 21 | 1.2E−05 | 44.63 |
| 5 | P19835 | CEL | Bile salt-activated lipase | K.LGLLGDSVDIFK.G | 22 | 4.5E−06 | 39.95 |
| 6 | P07478 | PRSS2 | Trypsin-2 | R.TLDNDILLIK.L | 23 | 3.5E−06 | 36 |
| 7 | P15085 | CPA1 | Carboxypeptidase A1 | K.TEPVPDQDELDQLSK.A | 24 | 2.4E−06 | 35.02 |
| 8 | Q13087 | PDIA2 | Protein disulfide-isomerase A2 | K.NFEQVAFDETK•N | 25 | 3.4E−05 | 30.06 |
| 9 | P07477 | PRSS1 | Trypsin-1 | K.TLNNDIMLIK.L | 26 | 3.3E−05 | 28.44 |
| 10 | P09210 | GSTA2 | Glutathione S-transferase A2 | K.LALIQEK.T | 27 | 9.0E−05 | 10.78 |
| 11 | O43175 | PHGDH | D-3-phosphoglycerate dehydrogenase | K.TLGILGLGR.I | 28 | 1.6E−06 | 10.27 |
| 12 | Q13310 | PABPC4 | Polyadenylate-binding protein 4 | K.SGVGNVFIK•N | 29 | 8.4E−08 | 9 |
| 13 | P07237 | P4HB | Protein disulfide-isomerase | K.VDATEESDLAQQYGVR.G | 30 | 1.1E−07 | 7.62 |
| 14 | P43307 | SSR1 | Translocon-associated protein subunit alpha | K.GEDFPANNIVK•F | 31 | 7.4E−07 | 7.21 |
| 15 | P16989 | YBX3 | Y-box-binding protein 3 | K.GAEAANVTGPDGVPVEGSR.Y | 32 | 9.0E−07 | 6.36 |
| 16 | Q9P2E9 | RRBP1 | Ribosome-binding protein 1 | K.LLATEQEDAAVAK•S | 33 | 1.8E−06 | 5.5 |
| 17 | Q96AG4 | LRRC59 | Leucine-rich repeat-containing protein 59 | K.LQQLPADFGR.L | 34 | 3.9E−05 | 5.21 |
| 18 | P13667 | PDIA4 | Protein disulfide-isomerase A4 | K.VEGFPTIYFAPSGDK•K | 35 | 1.9E−06 | 4.82 |
| 19 | P11021 | HSPA5 | 78 kDa glucose-regulated protein | K.NQLTSNPENTVFDAK.R | 36 | 5.6E−07 | 4.5 |
| 20 | Q9Y4L1 | HYOU1 | Hypoxia up-regulated protein 1 | K.AANSLEAFIFETQDK.L | 37 | 9.3E−05 | 4.23 |
| 21 | O94760 | DDAH1 | N(G), N(G)-dimethylarginine | R.ALPESLGQHALR.S | 38 | 4.4E−07 | 3.66 |
| | | | dimethylaminohydrolase 1 | | | | |
| 22 | P24534 | EEF1B2 | Elongation factor 1-beta | K.YGPADVEDTTGSGATDSK.D | 39 | 2.0E−04 | 3.41 |
| 23 | P30086 | PEBP1 | Phosphatidylethanolamine-binding protein 1 | K.LYEQLSGK.— | 40 | 1.4E−05 | 3.36 |
| 24 | P63220 | RPS21 | 40S ribosomal protein S21 | K.DHASIQMNVAEVDK•V | 41 | 4.7E−05 | 3.34 |
| 25 | P61247 | RPS3A | 40S ribosomal protein S3a | K.TTDGYLLR.L | 42 | 9.9E−06 | 3.34 |
| 26 | P62263 | RPS14 | 40S ribosomal protein S14 | K.TPGPGAQSALR.A | 43 | 2.9E−06 | 3.12 |
| 27 | P30050 | RPL12 | 60S ribosomal protein L12 | K.IGPLGLSPK•K | 44 | 1.8E−04 | 2.97 |
| 28 | Q92734 | TFG | Protein TFG | K.LLSNDEVTIK•Y | 45 | 1.9E−05 | 2.43 |

Abbreviations: NC, normal controls; PC, pancreatic cancer; PRM, parallel reaction monitoring.

TABLE 3
Clinicopathological variables stratified by BASP1 and WT1 expression.

| | BASP1 cohort | | | | | | | WT1 cohort | | | | | | |
|---|---|---|---|---|---|---|---|---|---|---|---|---|---|---|
| | Total | | Negative | | Positive | | | Total | | Low | | High | | |
| Factors | N = 141 | | N = 15 | | N = 126 | | p value | N = 139 | | N = 77 | | N = 62 | | p value |
| Age (>65 years) | 93 | (66) | 9 | (60) | 84 | (66.7) | 0.58 | 91 | (65.5) | 54 | (70.1) | 37 | (59.7) | 0.214 |
| Female gender | 67 | (47.5) | 7 | (46.7) | 60 | (47.6) | 1 | 68 | (48.9) | 35 | (45.5) | 33 | (53.2) | 0.397 |
| BMI (>25 kg/m²) | 57 | (42.9) | 9 | (60) | 48 | (40.7) | 0.175 | 56 | (42.7) | 30 | (41.7) | 26 | (44.1) | 0.86 |
| Smoking history | 66 | (47.1) | 6 | (40) | 60 | (48) | 0.596 | 65 | (47.1) | 38 | (50) | 27 | (43.5) | 0.495 |
| Diabetes mellitus | 33 | (23.6) | 1 | (7.1) | 32 | (25.4) | 0.188 | 33 | (23.9) | 20 | (26) | 13 | (21.3) | 0.553 |
| Symptoms at diagnosis | 132 | (96.4) | 14 | (100) | 118 | (95.9) | 1 | 130 | (96.3) | 73 | (96.1) | 57 | (96.6) | 1 |
| Tumor location (head) | 117 | (83) | 15 | (100) | 102 | (81) | 0.074 | 116 | (83.5) | 63 | (81.8) | 53 | (85.5) | 0.649 |
| Tumor size (>2 cm) | 118 | (84.3) | 13 | (86.7) | 105 | (84) | 1 | 115 | (83.3) | 64 | (83.1) | 51 | (83.6) | 1 |
| T-stage (≥T2) | 122 | (87.1) | 14 | (93.3) | 108 | (86.4) | 0.693 | 119 | (86.2) | 66 | (85.7) | 53 | (86.9) | 1 |
| N-stage (≥N1) | 106 | (76.3) | 10 | (66.7) | 96 | (77.4) | 0.349 | 103 | (75.2) | 62 | (81.6) | 41 | (67.2) | 0.073 |
| AJCC 8th edition (≥IIA) | 114 | (82) | 12 | (80) | 102 | (82.3) | 0.734 | 111 | (81) | 66 | (86.8) | 45 | (73.8) | 0.078 |
| Histological grade (≥3) | 82 | (59) | 10 | (66.7) | 72 | (58.1) | 0.589 | 83 | (60.6) | 47 | (61.8) | 36 | (59) | 0.86 |
| Resection margin (≥R1) | 55 | (39.3) | 6 | (40) | 49 | (39.2) | 1 | 53 | (38.4) | 28 | (36.4) | 25 | (41) | 0.601 |
| Adjuvant chemotherapy | 115 | (84.6) | 12 | (80) | 103 | (85.1) | 0.703 | 112 | (83.6) | 61 | (83.6) | 51 | (83.6) | 1 |
| Recurrence of disease | 102 | (79.7) | 12 | (85.7) | 90 | (78.9) | 0.734 | 102 | (81) | 55 | (78.6) | 47 | (83.9) | 0.5 |

Data were incomplete for some variables. Abbreviations: AJCC, American joint committee on cancer; BMI, body mass index; N-stage, nodal stage; T-stage, tumor stage.

TABLE 4
Univariable and multivariable Cox regression analyses of overall survival.

| | BASP1 cohort | | | | WT1 cohort | | | |
|---|---|---|---|---|---|---|---|---|
| | Univariable HR | | Multivariable HR | | Univariable HR | | Multivariable HR | |
| Variables | (95% CI) | p value | (95% CI) | p value | (95% CI) | p value | (95% CI) | p value |
| Age (>65 years) | 1.028 (0.682-1.547) | 0.896 | | | 1.035 (0.685-1.565) | 0.871 | | |
| Female gender | 0.785 (0.530-1.162) | 0.227 | | | 0.769 (0.519-1.139) | 0.19 | | |
| BMI (>25 kg/m²) | 1.346 (0.897-2.020) | 0.151 | | | 1.273 (0.846-1.914) | 0.247 | | |
| Smoking history | 1.633 (1.100-2.422) | 0.015* | 1.626 (1.071-2.471) | 0.023* | 1.664 (1.116-2.480) | 0.012* | 1.727 (1.128-2.644) | 0.012* |
| Diabetes mellitus | 0.851 (0.526-1.377) | 0.511 | | | 0.854 (0.522-1.395) | 0.527 | | |
| Symptoms at | 0.353 (0.128-0.973) | 0.044* | 0.404 (0.143-1.142) | 0.087 | 0.361 (0.131-0.995) | 0.049* | 0.459 (0.163-1.294) | 0.141 |
| diagnosis | | | | | | | | |
| Tumor location (head) | 0.667 (0.394-1.128) | 0.131 | | | 0.657 (0.389-1.110) | 0.117 | | |
| Tumor size (>2 cm) | 1.107 (0.663-1.849) | 0.697 | | | 1.082 (0.654-1.788) | 0.759 | | |
| T-stage (≥T2) | 1.173 (0.684-2.011) | 0.562 | | | 1.139 (0.673-1.927) | 0.629 | | |
| N-stage (≥N1) | 1.472 (0.916-2.366) | 0.11 | | | 1.454 (0.911-2.321) | 0.117 | | |
| AJCC 8th edition (≥IIA) | 1.443 (0.855-2.436) | 0.17 | | | 1.421 (0.852-2.370) | 0.179 | | |
| Histological grade (≥3) | 1.536 (1.018-2.317) | 0.041* | 1.647 (1.077-2.518) | 0.021* | 1.564 (1.033-2.368) | 0.035* | 1.696 (1.105-2.601) | 0.016* |
| Resection margin | 1.479 (0.986-2.219) | 0.058 | | | 1.506 (0.996-2.277) | 0.052 | | |
| (≥R1) | | | | | | | | |
| Adjuvant | 0.713 (0.431-1.180) | 0.188 | | | 0.712 (0.435-1.166) | 0.177 | | |
| chemotherapy | | | | | | | | |
| BASP1 (positive) | 0.523 (0.297-0.921) | 0.025* | 0.468 (0.257-0.852) | 0.013* | | | | |
| WT1 (high) | | | | | 1.561 (1.047-2.328) | 0.029* | 1.636 (1.083-2.473) | 0.019* |

Abbreviations: AJCC, American joint committee on cancer; BMI, body mass index; CI, confidence interval; HR, Hazard ratio; N-stage, nodal stage; T-stage, tumor stage. Variables with p < .05 are marked with asterisk (*), variables with p < .05 in univariable analysis were included in multivariable analysis.

### Discussion

As described above, particularly in relation to the examples described above in Tables 1-4, FIGS. 3-7, and the accompanying description, the MS discovery and verification phases showed that BASP1 protein was significantly overexpressed in pancreatic cancer tissues compared to healthy pancreas. Additionally, pancreatic cancer patients with high BASP1 expression levels in tumor tissue showed a significantly enhanced benefit from adjuvant chemotherapy. Additionally in this example, bioinformatic analyses demonstrated that BASP1 shares a close interaction network with WT1 in pancreatic cancer. WT1 is a zinc finger transcription factor, which is a confirmed oncogenic factor.

### Detection of Pancreatic Cancer Using Biomarkers

Carbohydrate antigen 19-9 (CA 19-9) is the sole blood-based biomarker approved by the FDA for clinical management of pancreatic cancer. However, CA 19-9 has a limited sensitivity (79%) and specificity (82%) for diagnosis of pancreatic cancer. For example, CA 19-9 levels can be elevated in several benign conditions and 5-7% of the population who are Lewis antigen negative do not express CA 19-9. Hence, CA 19-9 generally is not recommended as a screening test, but only for disease monitoring during treatment. Thus, as explained above, new markers are needed to enhance pancreatic cancer diagnosis, preferably by non-invasive methods. As will be described below, proteomic technology may be used to identify blood-based biomarkers that can aid in the detection of early-stage pancreatic cancer. These proteins can be combined with CA 19-9 to enhance diagnostic performance and they can be measured as an inexpensive, accurate and portable method of detecting pancreatic cancer.

As explained in the non-limiting example above, a number of proteins relevant to distinguishing pancreatic cancer tissue (fresh-frozen) versus normal controls were identified (2950 quantified proteins, see Table A). Additionally, certain combinations of differentially expressed biomarkers may be used to more easily distinguish between healthy tissue and pancreatic cancer, using biomarkers collected from a particular human source such as blood, serum, plasma, healthy or non-healthy tissue. In brief, 300 samples were tested, including 100 pancreatic cancer samples and 200 healthy samples.

From the study, the following 18 protein markers, also described above in relation to FIG. 1 and examples of a lateral flow assay detection system, in patients' blood were found to be particularly strong indicators of pancreatic cancer having the associated AUCs:

TABLE 5
Area under the Curve (AUC).

| | PC vs |
|---|---|
| | Healthy |
| Biomarker | (AUC) |
| A1AT (Alpha-1 antitrypsin; SERPINA1) | 0.884 |
| CA19-9 (Carbohydrate antigen 19-9) | 0.876 |
| GGT (Gamma-glutamyl transpeptidase) | 0.865 |
| C3 (Complement component 3) | 0.850 |
| C2 (Complement C2) | 0.836 |
| Ceruloplasmin | 0.800 |
| SAA (Serum amyloid A) | 0.788 |
| Haptoglobin | 0.779 |
| AGP1 (Alpha-1-acid glycoprotein 1) | 0.768 |
| C1 inhib (C1-inhibitor, C1-inh, C1 esterase inhibitor) | 0.724 |
| TNF-a (Tumor necrosis factor alpha) | 0.719 |
| ApoA1 (Apolipoprotein A1) | 0.716 |
| IGFB3 (IGFBP-3, Insulin-Like Growth Factor Binding | 0.693 |
| Protein 3) | |
| IGF1 (IGF-1, Insulin-like growth factor 1) | 0.687 |
| COMP (cartilage oligomeric matrix protein) | 0.665 |
| Calprotectin | 0.622 |
| CCK18 (caspase-cleaved cytokeratin-18) | 0.611 |
| Properdin | 0.510 |

An analysis of all 18 markers provides a diagnostic accuracy of 98.6% against healthy controls. Consequently, in certain examples of a method for detecting pancreatic cancer, some or all of these biomarkers may be used.

As described above in relation to FIG. 1, a patient's blood may be tested using a lateral flow assay or equivalent test, which may be a less expensive and more widely available means for testing for pancreatic cancer. However, as described above, lateral flow assays may be limited to detection of a certain number of markers to maintain affordability or due to other technical reasons. Therefore, as described above, further analysis was carried out to determine a reduced set of markers for use in a lateral flow assay or equivalent.

The analysis was conducted using the statistical programming language R. Spearman's rank correlation between each pair of variables was calculated using the cor function in base R to identify any potential issues with multicollinearity prior to calculating any models. No correlations above 70% were observed. The performance of each individual biomarker, in the form of Receiver Operator Characteristic (ROC) curves, was calculated using the R function pROC for a variety of comparisons between the diagnostic groups listed above. For multivariate analysis, logistic regressions were conducted using the R function lrm. This was done for the comparison Pancreatic Cancer versus Healthy (PCvH), on all 12,615 possible combinations of the variables up to panels of five.

For each combination of variables for each of the above comparisons, the following values were calculated:
The p-value for the model
The p-value (using the Wald statistic) for each variable within the model
The Nagelkerke R squared statistic
The Area Under the ROC Curve (AUC)
The sensitivity at 80% specificity
The sensitivity at 90% specificity

and the results tabulated. Logistic Regression models were held to be valid if the p-value for the model, and the p-value for each variable within the model, were all less than 0.05. Models not meeting the above criteria were discarded, and the remainder ordered in decreasing order of AUC. 749 valid models were found, with AUCs ranging from 61.1% to 98.1%.

Cross-validation (k=3, n=100) was performed on these models by dividing into randomly selected thirds, training on two thirds and testing on the final third. This was done for each third in turn. This process was repeated 100 times and the AUC calculated. An average of these 300 AUCs was then computed as the cross-validated AUC. For each of these eleven models the cross-validated AUC was calculated to be between 0.44% and 1.20% lower than the AUC of the model for that panel calculated on all of the data, which is a good result for models trained on this kind of data. The high performance of the models does not therefore rely on overtraining.

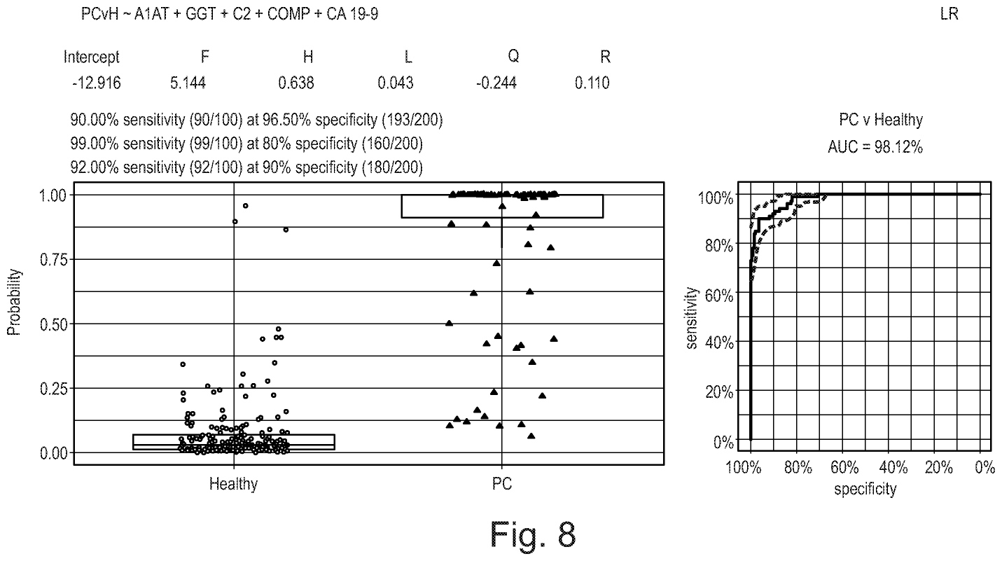

Referring to FIG. 8, example results are shown for 5 markers (A1AT, GGT, C2, COMP, CA 19-9), examining pancreatic cancer vs. healthy. Reduced sets may still provide a high degree of diagnostic accuracy against healthy controls. The following reduced set combinations of markers were found to be advantageous:

Any 2 or more marker combination comprising at least:
A1AT
CA 19-9

This combination provided a diagnostic accuracy of at least 95.8% against healthy controls.

Any 3 or more marker combination comprising at least:
A1AT
CA 19-9

At least one of Complement C2 and Complement component 3 This combination provided a diagnostic accuracy of at least 96.6% against healthy controls.

Any 4 or more marker combination comprising at least:
A1AT
CA 19-9
At least one of Complement C2 and Complement component 3
Cartilage oligomeric matrix protein

This combination provided a diagnostic accuracy of at least 97.5% against healthy controls.

Any 5 or more marker combination comprising at least:
A1AT
CA 19-9
At least one of Complement C2 and Complement component 3
Cartilage oligomeric matrix protein
At least one of Gamma-glutamyl transpeptidase, C1 inhibitor, and Serum amyloid A.

This combination provided a diagnostic accuracy of at least 97.5% against healthy controls.

5 marker combination
A1AT
GGT
C2
COMP
CA 19-9

This combination provided a diagnostic accuracy of 98.1% against healthy controls.

Any 4 or more marker combination comprising at least:
A1AT
CA 19-9
At least one of Complement C2 and Complement component 3
Gamma-glutamyl transpeptidase

This combination provided a diagnostic accuracy of at least 97.0% against healthy controls.

Any 5 or more marker combination comprising at least:
A1AT
CA 19-9
At least one of Complement C2 and Complement component 3
Gamma-glutamyl transpeptidase
At least one of C1 inhibitor, and Serum amyloid A.

This combination provided a diagnostic accuracy of at least 97.1% against healthy controls.

Any 3 or more marker combination comprising at least:
A1AT
CA 19-9
At least one of Gamma-glutamyl transpeptidase or Haptoglobin

This combination provided a diagnostic accuracy of at least 96.6% against healthy controls.

Any 4 or more marker combination comprising at least:
A1AT
CA 19-9
At least one of Gamma-glutamyl transpeptidase or Haptoglobin
Cartilage oligomeric matrix protein

This combination provided a diagnostic accuracy of at least 96.9% against healthy controls.

Any 5 or more marker combination comprising at least:
A1AT
CA 19-9
At least one of Gamma-glutamyl transpeptidase or Haptoglobin
Cartilage oligomeric matrix protein
At least one of Gamma-glutamyl transpeptidase, and Serum amyloid A. This combination provided a diagnostic accuracy of at least 97.5% against healthy controls.

Any 3 or more marker combination comprising at least:
A1AT
CA 19-9
Cartilage oligomeric matrix protein

This combination provided a diagnostic accuracy of at least 96.5% against healthy controls.

Any 2 or more marker combination comprising at least:
A1AT
At least one of Complement C2 and Complement component 3

This combination provided a diagnostic accuracy of at least 92.8% against healthy controls.

### Additional Biomarkers of Pancreatic Cancer

From a follow up study, the following 15 protein markers in patients' blood were found to be particularly strong indicators of pancreatic cancer:

TABLE 6
Biomarker

Claudin 18 (CLDN18)
Galectin 4 (LGALS4)
Matrix metalloproteinase 7 (MMP7)
Mucin 2 (MUC2)
Mucin 4 (MUC4)
Olfactomedin 4 (OLFM4)
Regenerating islet-derived protein 1-alpha (REG1A)
Regenerating islet-derived protein 1-beta (REG1B)
Serine protease inhibitor Kazal-type 1 (SPINK1)
Syncollin (SYCN)
Trefoil factor 1 (TFF1)
Carcinoembryonic antigen (CEA)
Lymphatic vessel endothelial hyaluronan receptor 1 (LYVE1)
Tenascin C (TNC)
Thrombospondin 2 (THBS2)

In some examples of a method for detecting pancreatic cancer, individual biomarkers listed in Tables 5 and 6, or a combination of biomarkers thereof, may be used. In some examples, 1 of the biomarkers may be used. In other examples, 2, 3, 4, 5, 6, 7, 9, 9, 10 or more of these markers may be used in combination as a method of detecting pancreatic cancer.

While the present description sets forth specific details of various examples, it will be appreciated that the description is illustrative only and should not be construed in any way as limiting. Furthermore, various applications of such examples and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference.

Some examples have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred examples and examples, it will be understood by those skilled in the art that the present inventions extend beyond the specifically disclosed examples to other alternative examples and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the examples may be made and still fall within the scope of the inventions. It should be understood that various features and aspects of the disclosed examples can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed examples described above. The limitations in the claims are to be interpreted broadly based on the language employed in the claims and not limited to the examples described in the present specification or during the prosecution of the application, which examples are to be construed as non-exclusive.

1. A pancreatic cancer detection device, comprising:
a solid surface comprising an antibody bound to the solid surface, the solid surface configured to indicate selective binding between the antibody and a target protein; and
wherein the antibody is configured to selectively bind to the target protein selected from the group consisting of Alpha-1 antitrypsin (A1AT), Alpha-1-acid glycoprotein 1 (AGP1), Apolipoprotein A1 (ApoA1), C1-inhibitor, Complement C2, Complement component 3, Carbohydrate antigen 19-9, Calprotectin, caspase-cleaved cytokeratin-18 (CCK18), Ceruloplasmin, cartilage oligomeric matrix protein, gamma-glutamyl transpeptidase, Haptoglobin, Insulin-like growth factor 1, Insulin-Like Growth Factor Binding Protein 3, Properdin, Serum amyloid A, and Tumor necrosis factor alpha (TNF-alpha).

2. The pancreatic cancer detection device of claim 1, wherein the solid surface comprises antibodies configured to selectively bind two target proteins selected from the group.

3. The pancreatic cancer detection device of claim 1, wherein the solid surface comprises antibodies configured to selectively bind three target proteins selected from the group.

4. The pancreatic cancer detection device of claim 1, wherein the solid surface comprises antibodies configured to selectively bind four target proteins selected from the group.

5. The pancreatic cancer detection device of claim 1, wherein the solid surface comprises antibodies configured to selectively bind five target proteins selected from the group.

6. The pancreatic cancer detection device of claim 1, wherein the solid surface comprises antibodies configured to selectively bind A1AT and Carbohydrate antigen 19-9.

7. The pancreatic cancer detection device of claim 6, wherein the solid surface comprises antibodies further configured to selectively bind at least one of Complement C2 and Complement component 3.

8. The pancreatic cancer detection device of claim 7, wherein the solid surface comprises antibodies further configured to selectively bind cartilage oligomeric matrix protein.

9. The pancreatic cancer detection device of claim 8, wherein the solid surface comprises antibodies further configured to selectively bind Gamma-glutamyl transpeptidase.

10. The pancreatic cancer detection device of claim 8, wherein the solid surface comprises antibodies further configured to selectively bind C1 inhibitor.

11. The pancreatic cancer detection device of claim 8, wherein the solid surface comprises antibodies further configured to selectively bind Serum amyloid A.

12. The pancreatic cancer detection device of claim 7, wherein the solid surface comprises antibodies further configured to selectively bind Gamma-glutamyl transpeptidase.

13. The pancreatic cancer detection device of claim 12, wherein the solid surface comprises antibodies further configured to selectively bind C1 inhibitor.

14. The pancreatic cancer detection device of claim 12, wherein the solid surface comprises antibodies further configured to selectively bind Serum amyloid A.

15. The pancreatic cancer detection device of claim 6, wherein the solid surface comprises antibodies further configured to selectively bind Gamma-glutamyl transpeptidase.

16. The pancreatic cancer detection device of claim 15, wherein the solid surface comprises antibodies further configured to selectively bind cartilage oligomeric matrix protein.

17. The pancreatic cancer detection device of claim 16, wherein the solid surface comprises antibodies further configured to selectively bind Serum amyloid A.

18. The pancreatic cancer detection device of claim 6, wherein the solid surface comprises antibodies further configured to selectively bind cartilage oligomeric matrix protein.

19. The pancreatic cancer detection device of claim 18, wherein the solid surface comprises antibodies further configured to selectively bind Serum amyloid A.

20. The pancreatic cancer detection device of claim 1, wherein the solid surface comprises antibodies further configured to selectively bind Carbohydrate antigen 19-9 and at least one of Complement C2 or Complement component 3.

21. The pancreatic cancer detection device of claim 1, wherein the solid surface comprises a lateral flow detection surface.

22. The pancreatic cancer detection device of claim 21, wherein the lateral flow detection surface comprises a lateral flow detection test trip.

23. The pancreatic cancer detection device of claim 1, wherein the solid surface comprises a plurality of microbeads or microspheres.

24. The pancreatic cancer detection device of claim 1, wherein indicating comprises visually indicating binding to a user.

25. A method of detecting pancreatic cancer, comprising:
collecting a biological sample from a subject;
contacting the biological sample with the pancreatic detection device of any one of the preceding claims; and
indicating a likelihood of an incidence of pancreatic cancer in the subject.

26. The method of claim 25, wherein the biological sample comprises whole blood.

27. The method of claim 26, wherein the biological sample comprises serum.

28. The method of claim 25, further comprising providing a report, the report indicating the likelihood of an incidence of pancreatic cancer in a subject.

29. A pancreatic cancer detection device, comprising:
a solid surface comprising an antibody bound to the solid surface, the solid surface configured to indicate selective binding between the antibody and a target protein; and
wherein the antibody is configured to selectively bind to the target protein selected from the group consisting of Claudin 18 (CLDN18), Galectin 4 (LGALS4), Matrix metalloproteinase 7 (MMP7), Mucin 2 (MUC2), Mucin 4 (MUC4), Olfactomedin 4 (OLFM4), Regenerating islet-derived protein 1-alpha (REG1A), Regenerating islet-derived protein 1-beta (REG1B), Serine protease inhibitor Kazal-type 1 (SPINK1), Syncollin (SYCN), Trefoil factor 1 (TFF1), Carcinoembryonic antigen (CEA), Lymphatic vessel endothelial hyaluronan receptor 1 (LYVE1), Tenascin C (TNC), and Thrombospondin 2 (THBS2).

30. The pancreatic cancer detection device of claim 29, wherein the solid surface comprises antibodies configured to selectively bind two target proteins selected from the group.

31. The pancreatic cancer detection device of claim 29, wherein the solid surface comprises antibodies configured to selectively bind three target proteins selected from the group.

32. The pancreatic cancer detection device of claim 29, wherein the solid surface comprises antibodies configured to selectively bind four target proteins selected from the group.

33. The pancreatic cancer detection device of claim 29, wherein the solid surface comprises antibodies configured to selectively bind five target proteins selected from the group.
